(19) United States
(12) Patent Application Publication
(10) Pub. No.: US 2009/0281075 A1
(43) Pub. Date: Nov. 12, 2009

(54) ISOMERIC PURINONES AND 1H-IMIDAZOPYRIDINONES AS PKC-THETA INHIBITORS

(75) Inventors: Andrew L. Roughton, Plainsboro, NJ (US); Yajing Rong, Dayton, NJ (US); Koc-Kan Ho, West Windsor, NJ (US); Michael J. Ohlmeyer, Plainsboro, NJ (US); David Diller, East Windsor, NJ (US); Ray Jui-Hsiang Chan, West Windsor, NJ (US); Gaifa Lai, Monmouth Junction, NJ (US)

Correspondence Address: KNOBBE MARTENS OLSON & BEAR LLP, 2040 MAIN STREET, FOURTEENTH FLOOR, IRVINE, CA, 92614, US

(73) Assignee: PHARMACOPEIA, INC., Cranbury, NJ (US)

(21) Appl. No.: 12/267,271

(22) Filed: Nov. 7, 2008

Related U.S. Application Data
(63) Continuation-in-part of application No. 11/701,632, filed on Feb. 2, 2007.
(60) Provisional application No. 60/774,492, filed on Feb. 17, 2006.
(60) Provisional application No. 60/986,450, filed on Nov. 8, 2007.

(51) Int. Cl. A61K 31/397; C07D 473/18; A61K 31/522; A61K 31/496; C07D 471/02; A61K 31/437; C07D 413/14; A61K 31/5377; A61P 37/06; A61P 29/00; A61P 35/00; A61P 3/10

(52) U.S. Cl. 514/210.21; 544/276; 514/263.37; 514/263.2; 514/252.16; 546/118; 514/303; 544/118; 514/234.2

(57) ABSTRACT

A chemical genus of purinones and 1H-imidazopyridinones, which are useful as PKCθ inhibitors, and their methods of use are disclosed. The genus is represented by the formula I:

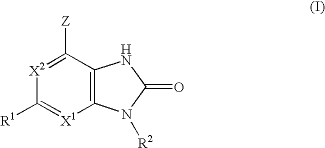

### RELATED APPLICATIONS

This application is a continuation in part of U.S. application Ser. No. 11/701,632 filed Feb. 2, 2007 (pending), which claims priority to U.S. Provisional Application No. 60/774,492 filed Feb. 17, 2006 (expired). Additionally, this application claims the benefit of priority from U.S. Provisional Application No. 60/986,450 filed Nov. 8, 2007.

### FIELD OF THE INVENTION

The present invention relates to a chemical genus of purinones and 1H-imidazopyridinones which are useful as PKCθ inhibitors.

### BACKGROUND OF THE INVENTION

Members of the protein kinase C (PKC) family of serine/threonine kinases play critical roles in the regulation of cellular differentiation and proliferation of diverse cell types. Ten mammalian members of PKC family have been identified and designated α, β, γ/δ, ε, ζ, η, θ, μ, and λ. The predicted structure of PKCθ displays the highest homology with members of the Ca²⁺ independent novel PKC subfamily, including PKCδ, ε, and η. PKCθ is most highly related to PKCδ.

PKCθ is expressed predominantly in lymphoid tissue and skeletal muscle. It has been shown that PKCθ is essential for T-cell receptor (TCR)-mediated T-cell activation but inessential during TCR-dependent thymocyte development. PKCθ, but not other PKC isoforms, translocates to the site of cell contact between antigen-specific T-cells and antigen presenting cells (APC), where it localizes with the TCR in the central core of the T-cell activation. PKCθ, but not the α, ε, or ζ isoenzymes, selectively activated a FasL promoter-reporter gene and upregulated the mRNA or cell surface expression of endogenous FasL. On the other hand, PKCδ and ε promoted T-cell survival by protecting the cells from Fas-induced apoptosis, and this protective effect was mediated by promoting p90Rsk-dependent phosphorylation of BAD. Thus, PKCθ appears to play a dual regulatory role in T-cell apoptosis.

The selective expression of PKCθ in T-cells and its essential role in mature T-cell activation establish that PKCθ inhibitors are useful for the treatment or prevention of disorders or diseases mediated by T lymphocytes, for example, autoimmune disease such as rheumatoid arthritis and lupus erythematosus, and inflammatory disease such as asthma and inflammatory bowel diseases.

PKCθ is identified as a drug target for immunosuppression in transplantation and autoimmune diseases (Isakov et al. (2002) Annual Review of Immunology, 20, 761-794). PCT Publication WO2004/043386 identifies PKCθ as a target for treatment of transplant rejection and multiple sclerosis. PKCθ also plays a role in inflammatory bowel disease (The Journal of Pharmacology and Experimental Therapeutics (2005), 313 (3), 962-982), asthma (WO 2005062918), and lupus (Current Drug Targets: Inflammation & Allergy (2005), 4 (3), 295-298).

In addition, PKCθ is highly expressed in gastrointestinal stromal tumors (Blay, P. et al. (2004) Clinical Cancer Research, 10, 12, Pt. 1), it has been suggested that PKCθ is a molecular target for treatment of gastrointestinal cancer (Wiedmann, M. et al. (2005) Current Cancer Drug Targets 5(3), 171). Thus, small molecule PKCθ inhibitors can be useful for treatment of gastrointestinal cancer.

Experiments conduced in PKCθ knock-out mice led to the conclusion that PKCθ inactivation prevented fat-induced defects in insulin signalling and glucose transport in skeletal muscle (Kim J. et al, 2004, The J. of Clinical Investigation 114 (6), 823). This data suggests that PKCθ is a potential therapeutic target for the treatment of type 2 diabetes, and hence small molecule PKCθ inhibitors can be useful for treating such disease.

Therefore, PKCθ inhibitors are useful in treatment of T-cell mediated diseases including autoimmune diseases such as rheumatoid arthritis, lupus erythematosus, and multiple sclerosis and inflammatory diseases such as asthma and inflammatory bowel disease. In addition, PKCθ inhibitors are useful in treatment of transplant rejection, gastrointestinal cancer, and diabetes.

### DESCRIPTION OF THE INVENTION

In its broadest sense, the invention relates to compounds of the formula I:

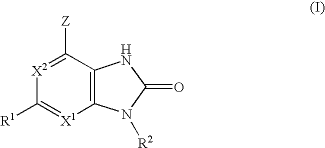

wherein:
X¹ and X² are independently chosen from N and CH, with the proviso that both
X¹ and X² are not CH;
R¹ is chosen from

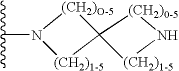

### and —NHR⁴;

R⁴ is chosen from
   -M-NR⁷R⁸ and

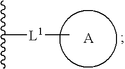

M is C₂-C₁₀ alkyl optionally substituted with —OH, with a proviso that —OH cannot be bonded to a carbon atom that is also bonded to N;
R⁷ and R⁸ are independently chosen from —H and C₁-C₄ alkyl;
A is chosen from carbocycle, substituted carbocycle, heterocycle and substituted heterocycle; and
L₁ is a C₁-C₁₀ alkyl optionally substituted with —OH, with the proviso that —OH cannot be bonded to a carbon atom that is also bonded to N;
R² is chosen from aryl, substituted aryl, arylalkyl, substituted arylalkyl, heteroaryl, substituted heteroaryl, hetroarylalkyl, and substituted heteroarylalkyl; and
Z is chosen from hydrogen, C₁-C₆ alkyl, cyano, hydroxyalkyl, carboxamido and haloalkyl.

In one embodiment, Z is hydrogen.

In another embodiment, R¹ is chosen from:

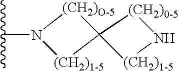

### and —NHR⁴;

wherein

R⁴ is chosen from

### -M-NR⁷R⁸,

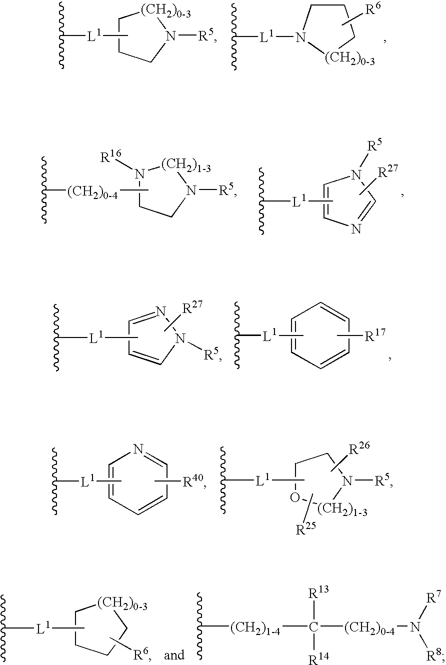

wherein

M is C₂-C₁₀ alkyl optionally substituted with —OH, with a proviso that —OH cannot be bonded to a carbon atom that is also bonded to N;

R⁷ and R⁸ are independently chosen from —H and C₁-C₄ alkyl; represents one, two or three residues independently chosen from —H, C₁-C₄ alkyl, haloalkyl, hydroxyalkyl, hydroxyaminoalkyl, alkoxyalkyl and aminoalkyl;

R⁶ represents one, two or three residues independently chosen from —H, C₁-C₄ alkyl, —OCH₃, hydroxy, amino, haloalkyl, acylamino, hydroxyalkyl, hydroxyaminoalkyl, hydroxyalkylamino, alkylamino, dialkylamino and aminoalkyl;

R¹³ and R¹⁴ are independently chosen from —H, —OH, and C₁-C₄ alkyl, with a proviso that —OH cannot be bonded to a carbon atom that is also bonded to N;

R¹⁶ is chosen from —H, C₁-C₄ alkyl, amino, and aminoalkyl;

R¹⁷ is chosen from C₁-C₄ alkyl, hydroxyalkyl, amino, aminoalkyl and optionally substituted heterocycloalkyl;

R⁴⁰ is chosen from —H, amino and lower alkyl;

R²⁵ is chosen from —H and lower alkyl;

R²⁶ is chosen from —H and oxo; and

R²⁷ is chosen from —H, C₁-C₄ alkyl, amino and alkylamino.

In a further embodiment, R⁴ is -M-NR⁷R⁸ wherein M is C₂-C₄ alkyl optionally substituted with hydroxy, and R⁷ and R⁸ are independently chosen from hydrogen and methyl.

In one aspect of the invention, X¹ and X² are both nitrogen. In another aspect, X¹ is nitrogen and X² is CH. In a further embodiment of the invention, X¹ is CH and X² is nitrogen.

In another embodiment of the invention, R² is chosen from

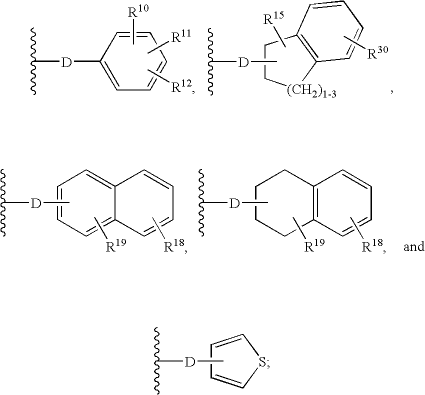

wherein
R¹⁰, R¹¹, and R¹² are independently chosen from —H, halogen, —OCH₃, —OCF₃, —CH₂OCF₃, —CF₃, —CN, alkylthio, —SO₂Me, —SCF₃, C₁-C₆ alkyl, substituted C₁-C₆ alkyl, C₁-C₄ alkenyl, C₁-C₄ alkynyl, C₁-C₄ acetate, C₁-C₄ alkylamino, carboxamide, substituted carboxamide, aryl, substituted aryl, heterocycle, substituted heterocycle, aryloxy, and substituted aryloxy;
R¹⁵ is chosen from hydroxyl and alkoxy;
R¹⁸ and R¹⁹ are each independently chosen from hydrogen, halogen, C₁-C₄ alkyl, cyano, C₁-C₄ alkynyl and trifluoromethoxy;
R³⁰ represents one or two residues independently chosen from hydrogen and halogen; and
D is C₀-C₆ alkyl. In an embodiment, for example, D is a C₁-C₆ linear or branched alkyl.

In a further aspect of the invention, R² is

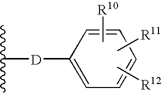

and D is C₀-C₃ alkyl. In yet another embodiment, D is methyl.

In a further embodiment, the invention relates to compounds of formula Ib:

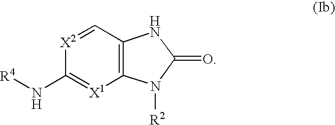

In another embodiment, the invention relates to compounds wherein R² is chosen from

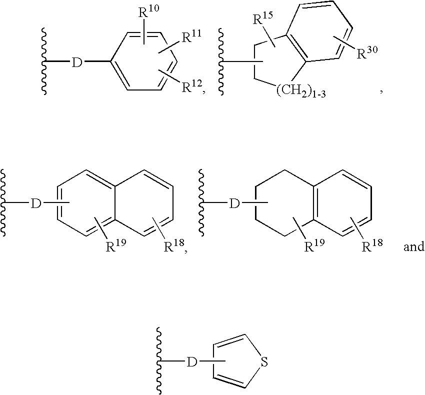

and D is C₀-C₃ alkyl. In yet another embodiment, R² is

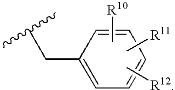

In still another aspect of the invention relating to compounds, R⁴ is chosen from

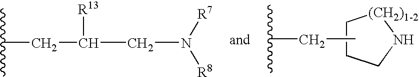

wherein R⁷ and R⁸ are independently chosen from —H and —CH₃ and R¹³ is chosen from —H and —OH.

In a further aspect of the invention, R⁴ is chosen from

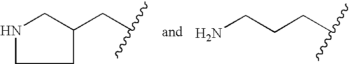

### and R² is

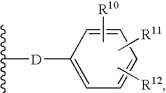

wherein D is a C₁-C₆ linear or branched alkyl; and R¹⁰, R¹¹, and R¹² are independently chosen from —H, halogen, —OCH₃, —OCF₃, —CH₂OCF₃, —CF₃, —CN, alkylthio, —SO₂Me, —SCF₃, C₁-C₆ alkyl, C₁-C₄ alkenyl, C₁-C₄ alkynyl, C₁-C₄ acetate, C₁-C₄ alkylamino, carboxamide, substituted carboxamide, aryl, substituted aryl, heterocycle, substituted heterocycle, aryloxy, and substituted aryloxy.

In a further embodiment of the invention relating to compounds, R⁴ is

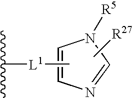

### and R² is

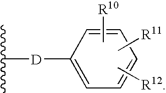

The invention also includes compounds in which L¹ is —CH₂; X¹ and X² are both nitrogen; Z is hydrogen; R⁵ is hydrogen; R²⁷ is methyl; R¹⁰ is chloro; and R¹² is —OCF₃.

Yet another embodiment of the invention relates to compounds in which R⁴ is

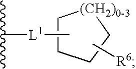

L¹ is —CH₂; and R⁶ is chosen from hydrogen, amino, aminoalkyl, hydroxyalkylamino, hydroxyl, acylamino and hydroxyalkyl. Still another embodiment of the invention relates to compounds in which R⁴ is

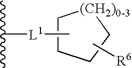

and R⁶ is selected from amino and hydroxyl.

A further embodiment of the invention relates to compounds in which R⁴ is selected from

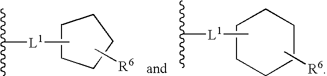

Yet another embodiment of the invention includes compounds wherein L¹ is CH₂, R² is

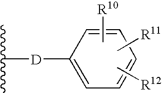

and R⁴ is selected from

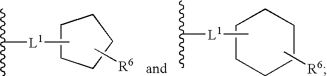

a further embodiment of the invention relates to compounds wherein R¹⁰ is —OCF₃, R¹¹ is halogen and R¹² is hydrogen.

In another aspect, the invention relates to compounds of formula II:

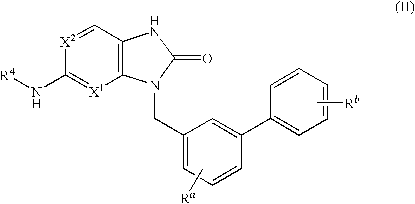

wherein

X¹ and X² are independently chosen from N and CH, with the proviso that both X¹ and X² are not CH;

R⁴ is chosen from

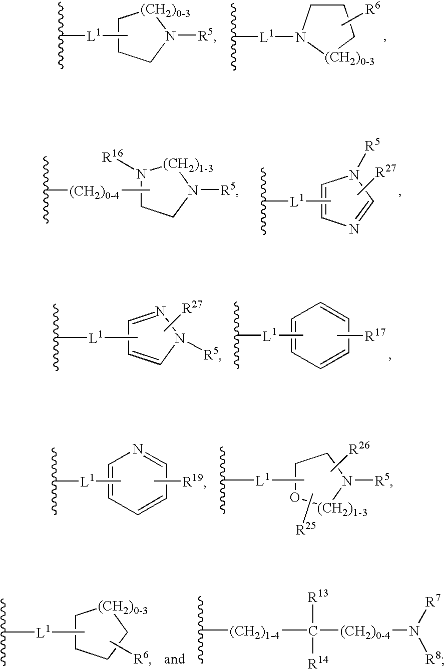

wherein

R⁵ represents one, two or three residues independently chosen from —H, C₁-C₄ alkyl, haloalkyl, hydroxyalkyl, hydroxyaminoalkyl, alkoxyalkyl and aminoalkyl;

R⁶ represents one, two or three residues independently chosen from —H, C₁-C₄ alkyl, —OCH₃, hydroxy, amino, haloalkyl, acylamino, hydroxyalkyl, hydroxyaminoalkyl, hydroxyalkylamino, alkylamino, dialkylamino and aminoalkyl;

R⁷ and R⁸ are independently chosen from —H and C₁-C₄ alkyl;

R¹³ and R¹⁴ are independently chosen from —H, —OH, and C₁-C₄ alkyl, with a proviso that —OH cannot be bonded to a carbon atom that is also bonded to N;

R¹⁶ is chosen from —H, C₁-C₄ alkyl, amino, and aminoalkyl;

R¹⁷ is chosen from —H, C₁-C₄ alkyl, hydroxyalkyl, amino, aminoalkyl and optionally substituted heterocycloalkyl;

R¹⁹ is chosen from —H, amino and lower alkyl;

R²⁵ is chosen from —H and lower alkyl;

R²⁶ is chosen from —H and oxo; and

R²⁷ is chosen from —H, C₁-C₄ alkyl, amino and alkylamino;

R^{a} represents one, two, or three residues independently chosen from —H, halogen, —OCH₃, —OCF₃, —CF₃, —CN, —SMe, —SO₂Me, —SCF₃, C₁-C₄ alkyl, amino, and aminoalkyl; and

R^{b} represents one, two, or three residues independently chosen from halogen, —OCH₃, —OCF₃, —CF₃, —CN, —SMe, —SO₂Me, —SCF₃, C₁-C₄ alkyl, carboxamide, alkoxyalkyl, amino, and aminoalkyl.

In a further embodiment, the invention relates to compounds of formula II wherein R^{a} represents one, two, or three residues independently chosen from —H, —OCF₃ and C₁-C₄ alkyl; and R^{b} represents one, two, or three residues independently chosen from hydrogen, halogen, C₁-C₄ alkyl, alkoxycarbonyl, carboxamide, alkoxyalkyl, amide, hydroxyalkyl and aminoalkyl. In yet another embodiment, the invention relates to compounds wherein R^{a} is chosen from C₁-C₄ alkyl and hydrogen and R^{b} is chosen from halogen and aminoalkyl. In another aspect, the invention relates to compounds wherein R^{a} is chosen from methyl and hydrogen and R^{b} is selected from fluorine, chlorine and —CH₂NH₂.

In one embodiment, the invention is directed to a pharmaceutical formulation comprising a compound of the invention and a pharmaceutically acceptable carrier.

In another aspect the invention relates to a method for treating a T-cell mediated disease including an autoimmune disease such as rheumatoid arthritis, lupus erythematosus, multiple sclerosis and psoriasis; inflammatory disease such as asthma and inflammatory bowel disease; or transplant rejection. The method comprises administering a therapeutically effective amount of a compound of formula I described herein, or a salt or solvate thereof.

In yet another embodiment, the invention is directed to a method of treating diabetes comprising administering a therapeutically effective amount of a compound of formula I, or a salt or solvate thereof.

In another embodiment, the invention is directed to a method of treating cancer, such as gastrointestinal cancer, comprising administering a therapeutically effective amount of a compound of formula I, or a salt or solvate thereof.

### DEFINITIONS

In this specification the terms and substituents are defined when introduced and retain their definitions throughout.

Alkyl is intended to include linear, branched, or cyclic hydrocarbon structures and combinations thereof. A combination would be, for example, cyclopropylmethyl. When not otherwise restricted, the term refers to alkyl of 10 or fewer carbons. Lower alkyl refers to alkyl groups of 1, 2, 3, 4, 5 and 6 carbon atoms. Examples of lower alkyl groups include methyl, ethyl, propyl, isopropyl, butyl, s- and t-butyl and the like. Preferred alkyl and alkylene groups are those of C₁₀ or below (e.g. C₁, C₂, C₃, C₄, C₅, C₆, C₇, C₈, C₉, C₁₀); most preferred are lower alkyl. Cycloalkyl is a subset of alkyl and includes cyclic hydrocarbon groups of 3, 4, 5, 6, 7, and 8 carbon atoms. Examples of cycloalkyl groups include c-propyl, c-butyl, c-pentyl, norbornyl, adamantyl and the like.

C₁ to Cₙ Hydrocarbon (e.g. C₁, C₂, C₃, C₄, C₅, C₆, C₇, C₈, C₉, C₁₀) includes alkyl, cycloalkyl, alkenyl, alkynyl, aryl and combinations thereof. Examples include benzyl, phenethyl, vinyl, allyl, cyclopropyl, cyclohexylmethyl, camphoryl and naphthylethyl. Hydrocarbon refers to any substituent comprised of hydrogen and carbon as the only elemental constituents. Saturated (C₁ to Cₙ) hydrocarbon is identical in meaning to (C₁ to Cₙ) alkyl or (C₁ to Cₙ) alkane as used herein. Whenever reference is made to C₀₋ₙ alkyl, (C₀ to Cₙ) alkyl, or (C₀ to Cₙ) alkane when number of carbon atoms is 0, a direct bond is implied.

Unless otherwise specified, the term “carbocycle” is intended to include ring systems in which the ring atoms are all carbon but of any oxidation state. Thus (C₃-C₁₀) carbocycle refers to such systems as cyclopropane, benzene and cyclohexene; (C₈-C₁₂) carbopolycycle refers to such systems as norbornane, decalin, indane and naphthalene. Carbocycle, not otherwise limited, refers to monocycles, bicycles and polycycles.

Alkoxy or alkoxyl refers to groups of from 1 to 8 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. Lower-alkoxy refers to groups containing one to four carbons. Similarly, aryloxy, cycloalkoxy and arylalkoxy are aryl, cycloalkyl and arylalkyl, respectively, bonded via an oxygen atom. For the purpose of this application, alkoxy and lower alkoxy include methylenedioxy and ethylenedioxy.

The term “halogen” or “halo” refers to fluorine, chlorine, bromine or iodine.

The terms “haloalkyl” and “haloalkoxy” mean alkyl or alkoxy, respectively, substituted with one or more halogen atoms. The terms “alkylcarbonyl” and “alkoxycarbonyl” mean —C(═O)alkyl or —C(═O)alkoxy, respectively.

Fluoroalkyl refers to alkyl residues in which one or more hydrogens have been replaced by fluorine. It includes perfluoroalkyl, in which all the hydrogens have been replaced by fluorine. Examples include fluoromethyl, difluoromethyl, trifluoromethyl, trifluoroethyl and pentafluoroethyl.

Oxaalkyl refers to alkyl residues in which one or more carbons (and their associated hydrogens) have been replaced by oxygen. Examples include methoxypropoxy, 3,6,9-trioxadecyl and the like. The term oxaalkyl is intended as it is understood in the art [see Naming and Indexing of Chemical Substances for Chemical Abstracts, published by the American Chemical Society, ¶196, but without the restriction of ¶127(a)], i.e. it refers to compounds in which the oxygen is bonded via a single bond to its adjacent atoms (forming ether bonds); it does not refer to doubly bonded oxygen, as would be found in carbonyl groups. Similarly, thiaalkyl and azaalkyl refer to alkyl residues in which one or more carbons has been replaced by sulfur or nitrogen, respectively. Examples include ethylaminoethyl and methylthiopropyl.

Acyl refers to formyl and to groups of 1, 2, 3, 4, 5, 6, 7 and 8 carbon atoms of a straight, branched or cyclic configuration, saturated, unsaturated and aromatic and combinations thereof, attached to the parent structure through a carbonyl functionality. One or more carbons in the acyl residue may be replaced by nitrogen, oxygen or sulfur as long as the point of attachment to the parent remains at the carbonyl. Examples include acetyl, benzoyl, propionyl, isobutyryl, t-butoxycarbonyl, benzyloxycarbonyl and the like. Lower-acyl refers to groups containing one to four carbons. The double bonded oxygen, when referred to as a substituent itself, is called “oxo”.

Aryl and heteroaryl mean (i) a phenyl group (or benzene) or a monocyclic 5- or 6-membered heteroaromatic ring containing 1-4 heteroatoms selected from O, N, or S; (ii) a bicyclic 9- or 10-membered aromatic or heteroaromatic ring system containing 0-4 heteroatoms selected from O, N, or S; or (iii) a tricyclic 13- or 14-membered aromatic or heteroaromatic ring system containing 0-5 heteroatoms selected from O, N, or S. As commonly understood, when referring to aryl as a substituent, it is intended that the point of attachment is a ring carbon of the aryl group (or ring carbon or heteroatom of the heteroaryl). For the purpose of the present invention, aryl and heteroaryl refer to systems in which at least one ring, but not necessarily all rings, are fully aromatic. Thus aromatic 6- to 14-membered carbocyclic rings include, e.g., benzene, naphthalene, indane, tetralin, benzocycloheptene and fluorene and the 5- to 10-membered aromatic heterocyclic rings include, e.g., imidazole, pyridine, indole, isoindoline, thiophene, benzopyranone, thiazole, furan, benzimidazole, quinoline, isoquinoline, tetrahydroisoquinoline, quinoxaline, tetrahydrocarboline, pyrimidine, pyrazine, tetrazole and pyrazole.

Arylalkyl means an alkyl residue attached to an aryl ring. As commonly understood, when referring to arylalkyl as a substituent, it is intended that the point of attachment is the alkyl group. Examples of arylalkyl are benzyl, phenethyl, phenylpropyl, naphthylethyl and the like. Heteroarylalkyl refers to a substituent in which a heteroaryl residue is attached to the parent structure through alkyl. Examples include, e.g., pyridinylmethyl, pyrimidinylethyl and the like. In one embodiment, the alkyl group of an arylalkyl or a heteroarylalkyl is an alkyl group of from 1 to 6 carbons.

The term “heterocycle” means a monocyclic, bicyclic or tricyclic residue with 1 to 13 carbon atoms and 1 to 5 heteroatoms chosen from the group consisting of nitrogen, oxygen and sulfur. The nitrogen and sulfur heteroatoms may optionally be oxidized, and the nitrogen heteroatom may optionally be quaternized. Heterocycles also include spiroheterocycles. Unless otherwise specified, a heterocycle may be non-aromatic or aromatic. The heterocycle may be fused to an aromatic hydrocarbon radical. Suitable examples include pyrrolyl, pyridinyl, pyrazolyl, triazolyl, pyrimidinyl, pyridazinyl, oxazolyl, thiazolyl, imidazolyl, indolyl, thiophenyl, furanyl, tetrazolyl, 2-pyrrolinyl, 3-pyrrolinyl, pyrrolindinyl, 1,3-dioxolanyl, imidazolinyl, imidazolidinyl, pyrazolinyl, pyrazolidinyl, isoxazolyl, isothiazolyl, 1,2,3-oxadiazolyl, 1,2,3-triazolyl, 1,3,4-thiadiazolyl, 2H-pyranyl, 4H-pyranyl, piperidinyl, 1,4-dithianyl, thiomorpholinyl, pyrazinyl, piperazinyl, 1,3,5-triazinyl, 1,2,5-trithianyl, benzo(b)thiophenyl, benzimidazolyl, quinolinyl, and the like. A nitrogen heterocycle is a heterocycle containing at least one nitrogen in the ring; it may contain additional nitrogens, as well as other heteroatoms. Examples include piperidine, piperazine, morpholine, pyrrolidine and thiomorpholine. It is to be noted that heteroaryl is a subset of heterocycle in which the heterocycle is aromatic; examples include pyridine, pyrrole and thiazole. Examples of heterocyclyl residues additionally include piperazinyl, 2-oxopiperazinyl, 2-oxopiperidinyl, 2-oxo-pyrrolidinyl, 2-oxoazepinyl, azepinyl, 4-piperidinyl, pyrazolidinyl, imidazolyl, imidazolinyl, imidazolidinyl, pyrazinyl, oxazolidinyl, isoxazolidinyl, thiazolidinyl, isothiazolyl, quinuclidinyl, isothiazolidinyl, benzimidazolyl, thiadiazolyl, benzopyranyl, benzothiazolyl, tetrahydrofuryl, tetrahydropyranyl, thienyl, benzothienyl, thiamorpholinyl, thiamorpholinylsulfoxide, thiamorpholinylsulfone, oxadiazolyl, triazolyl and tetrahydroquinolinyl.

Alkylthio means a lower alkyl group bound to a core structure via a sulfur atom, e.g. methylthio (—SCH₂), propylthio (—SCH₂CH₂CH₃).

Alkylamino means one lower alkyl group bound to a core structure via an amino group, e.g., methylamino, ethylamino, butylamino, etc. Dialkylamino means two lower alkyl groups bound to a core structure via an amino group. Each lower alkyl group replaces one hydrogen on the nitrogen of the amino, e.g., dimethylamino (—NCH₃CH₃).

Aminoalkyl means an amino group bound to a core structure via an alkyl group, e.g., aminomethyl, aminoethyl, aminopenthyl, etc. The alkyl group, as defined above, could be straight or branched and, therefore, an aminoalkyl includes, e.g., —CH₂CH₂CH(CH₃)CH₂NH₂, —CH₂C(CH₃)₂CH₂NH₂, etc. Alkylaminoalkyl means a secondary amine bound to a core structure via an alkyl group, e.g., —CH₂CH₂NHCH₃, —CH₂CH₂CH₂NHCH₂CH₃, etc. Dialkylaminoalkyl means a tertiary amine bound to a core structure via an alkyl group, e.g., —CH₂N(CH₃)₂, —CH2CH2CH₂N(CH₃)CH₂CH₃, etc.

It will be understood by the person of skill that hydroxyalkyl, hydroxyaminoalkyl and aminoalkyl structures will be unstable when directly attached to a heteroatom, for instance, in the case of hydroxymethyl bonded to a ring nitrogen.

Substituted alkyl, aryl, aryloxy, cycloalkyl, carbocycle, heterocyclyl etc. refer to alkyl, aryl, aryloxy, cycloalkyl, carbocycle, or heterocyclyl wherein up to three H atoms in each residue are replaced with halogen, alkyl, haloalkyl, haloalkoxy, haloalkoxyalkyl, haloalkylthio, perfluoroloweralkoxy, hydroxy, loweralkoxy (which for the purpose of the present disclosure includes methylene dioxy and ethylene dioxy), oxaalkyl, carboxy, carboalkoxy (also referred to as alkoxycarbonyl[-C(═O)O-alkyl]), carboxamido([—C(═O)NH₂]), alkylaminocarbonyl[-C(═O)NH-alkyl]), alkoxycarbonylamino[HNC(═O)O-alkyl], hydroxyaminoalkylcarbonyl, hydroxyalkylaminocarbonyl, acyl, alkoxyalkyl, benzenesulfonyl, cyano, carbonyl, nitro, amino, hydroxyalkyl, alkylamino, dialkylamino, aminoalkyl, (alkyl)(aryl)aminoalkyl, alkylaminoalkyl (including cycloalkylaminoalkyl), dialkylaminoalkyl, dialkylaminoalkoxy, heterocyclylalkoxy, mercapto, alkylthio, alkylsulfinyl, alkylsulfonyl, acylamino, acylaminoalkyl, acylaminoalkoxy, amidino, alkoxycarbonylamino, acetoxy, sulfoxide, sulfone, sulfonylamino, sulfonamido, aryl, phenyl, heterocyclyl, hydroxyimino, alkoxyimino, aminosulfonyl, trityl, amidino, guanidino, ureido, alkylaminosulfonyl, alkylureido, benzyloxyphenyl, benzyl, heteroaryl, heterocyclylalkyl, phenoxy, benzyloxy, or heteroaryloxy. Haloakyl refers to an alkyl group in which one or more hydrogens are replaced by halogen, for example, trifluoromethyl, trifluoromethoxy, trichloroethyl, and difluoromethyl. “Oxo” is also included among the substituents referred to in “substituted”; it will be appreciated by persons of skill in the art that, because oxo is a divalent radical, there are circumstances in which it will not be appropriate as a substituent (e.g. on phenyl). In one embodiment, 1, 2 or 3 hydrogen atoms are replaced with a specified radical. As used herein, the term “optionally substituted” may be used interchangeably with “unsubstituted or substituted”.

As used herein, reference to “treatment” or “treating” a patient are intended to include prophylaxis. The terms include amelioration, prevention and relief from the symptoms and/or effects associated with these disorders. The terms “preventing” or “prevention” as used herein refer to administering a medicament beforehand to forestall or obtund an acute episode or, in the case of a chronic condition, to diminish the likelihood or seriousness of the condition. Persons of ordinary skill in the medical art (to which the present use claims are directed) recognize that the term “prevent” is not an absolute term. In the medical art it is understood to refer to the prophylactic administration of a drug to diminish the likelihood or seriousness of a condition, and this is the sense intended.

It will be recognized that the compounds of this invention can exist in radiolabeled form, i.e., the compounds may contain one or more atoms containing an atomic mass or mass number different from the atomic mass or mass number usually found in nature. Radioisotopes of hydrogen, carbon, phosphorous, sulfur, and fluorine include ³H, ¹⁴C, ³²P, ³⁵S, and ¹⁸F, respectively. Compounds that contain those radioisotopes and/or other radioisotopes of other atoms are within the scope of this invention. Tritiated, i.e. ³H, and carbon-14, i.e., ¹⁴C, radioisotopes are particularly preferred for their ease in preparation and detectability. Radiolabeled compounds of this invention and prodrugs thereof can generally be prepared by methods well known to those skilled in the art. Conveniently, such radiolabeled compounds can be prepared by carrying out the procedures disclosed in the Examples and Schemes by substituting a readily available radiolabeled reagent for a non-radiolabeled reagent.

As used herein, and as would be understood by the person of skill in the art, the recitation of “a compound” is intended to include salts, solvates and inclusion complexes of that compound as well as any stereoisomeric form, or a mixture of any such forms of that compound in any ratio.

The term “solvate” refers to a compound of Formula I in the solid state, wherein molecules of a suitable solvent are incorporated in the crystal lattice. A suitable solvent for therapeutic administration is physiologically tolerable at the dosage administered. Examples of suitable solvents for therapeutic administration are ethanol and water. When water is the solvent, the solvate is referred to as a hydrate. In general, solvates are formed by dissolving the compound in the appropriate solvent and isolating the solvate by cooling or using an antisolvent. The solvate is typically dried or azeotroped under ambient conditions. Inclusion complexes are described in Remington: The Science and Practice of Pharmacy 19^{th} Ed. (1995) volume 1, page 176-177, which is incorporated herein by reference. The most commonly employed inclusion complexes are those with cyclodextrins, and all cyclodextrin complexes, natural and synthetic, are specifically encompassed within the claims.

The present invention includes compounds of formula I in the form of salts. Suitable salts include those formed with both organic and inorganic acids. Such salts will normally be pharmaceutically acceptable, although non-pharmaceutically acceptable salts may be of utility in the preparation and purification of the compound in question. The term “pharmaceutically acceptable salt” refers to salts prepared from pharmaceutically acceptable non-toxic acids or bases including inorganic acids and bases and organic acids and bases. When the compounds of the present invention are basic, salts may be prepared from pharmaceutically acceptable non-toxic acids including inorganic and organic acids. Suitable pharmaceutically acceptable acid addition salts for the compounds of the present invention include acetic, benzenesulfonic (besylate), benzoic, camphorsulfonic, carbonic, citric, ethanedisulfonic, ethanesulfonic, ethylenediaminetetraacetic, fumaric, glucoheptonic, gluconic, glutamic, hydrobromic, hydrochloric, hydroiodic, hydroxynaphthoic, isethionic, lactic, lactobionic, laurylsulfonic, maleic, malic, mandelic, methanesulfonic, mucic, naphthylenesulfonic, nitric, pamoic, pantothenic, phosphoric, polygalacturonic, salicylic, stearic, succinic, sulfuric, tannic, tartaric acid, teoclatic, p-toluenesulfonic, and the like. When the compounds contain an acidic side chain, suitable pharmaceutically acceptable base addition salts for the compounds of the present invention include, but are not limited to, metallic salts made from aluminum, calcium, lithium, magnesium, potassium, sodium and zinc or organic salts made from lysine, arginine, N,N′-dibenzylethylenediamine, chloroprocaine, choline, diethanolamine, ethylenediamine, meglumine (N-methylglucamine) and procaine.

Some of the compounds described herein contain one or more asymmetric centers and may thus give rise to enantiomers, diastereomers, and other stereoisometric forms which may be defined in terms of absolute stereochemistry as (R) — or (S)—. The present invention is meant to include all such possible diastereomers as well as their racemic and optically pure forms. Optically active (R)- and (S)-isomers may be prepared using homo-chiral synthons or homo-chiral reagents, or optically resolved using conventional techniques. When the compounds described herein contain olefinic double bonds or other centers of geometric asymmetry, and unless specified otherwise, it is intended to include both (E)- and (Z)-geometric isomers. Likewise, all tautomeric forms are intended to be included.

The graphic representations of racemic, ambiscalemic and scalemic or enantiomerically pure compounds used herein are taken from Maehr J. Chem. Ed. 62, 114-120 (1985): solid and broken wedges are used to denote the absolute configuration of a chiral element; wavy lines indicate disavowal of any stereochemical implication which the bond it represents could generate; solid and broken bold lines are geometric descriptors indicating the relative configuration shown but denoting racemic character; and wedge outlines and dotted or broken lines denote enantiomerically pure compounds of indeterminate absolute configuration. For example, the graphic representation

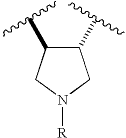

indicates either, or both, of the two trans enantiomers

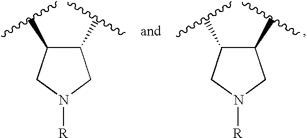

while the graphic representation

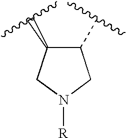

indicates a single diastereomer of unknown stereochemistry.

The terminology related to “protecting”, “deprotecting” and “protected” functionalities is well understood by persons of skill in the art and can be used in the context of processes which involve sequential treatment with a series of reagents. In that context, a protecting group refers to a group which is used to mask a functionality during a process step in which it would otherwise react, but in which reaction is undesirable. The protecting group prevents reaction at that step, but may be subsequently removed to expose the original functionality. The removal or “deprotection” occurs after the completion of the reaction or reactions in which the functionality would interfere. Thus, when a sequence of reagents is specified a person of ordinary skill can readily envision those groups that would be suitable as “protecting groups”.

For purposes of the specification of this application, the term “formulation” is used interchangeably with the term “composition.”

While it may be possible for the compounds of the invention to be administered as the raw chemical, it is possible to present them as a formulation. According to a further aspect, the present invention provides a formulation comprising a compound of the invention (or a pharmaceutically acceptable salt or solvate thereof) with one or more pharmaceutically acceptable carriers thereof and optionally one or more other therapeutic ingredients. The carrier(s) must be “acceptable” in the sense of being compatible with the other ingredients of the formulation and not deleterious to the recipient thereof.

The compositions may include a “pharmaceutically acceptable inert carrier”, and this expression is intended to include one or more inert excipients, which include starches, polyols, granulating agents, microcrystalline cellulose, diluents, lubricants, binders, disintegrating agents, and the like. If desired, tablet dosages of the disclosed compositions may be coated by standard aqueous or nonaqueous techniques. “Pharmaceutically acceptable carrier” also encompasses controlled release means.

Compositions of the present invention may also optionally include other therapeutic ingredients, anti-caking agents, preservatives, sweetening agents, colorants, flavors, desiccants, plasticizers, dyes, and the like. Any such optional ingredient must, of course, be compatible with the compound of the invention to insure the stability of the formulation.

The formulations include those suitable for oral, parenteral (including subcutaneous, intradermal, intramuscular, intravenous and intraarticular), rectal and topical (including dermal, buccal, sublingual and intraocular) administration. The most suitable route may depend upon the condition and disorder of the recipient. The formulations may conveniently be presented in unit dosage form and may be prepared by any of the methods well known in the art of pharmacy. All methods include the step of bringing into association a compound of the invention [or a pharmaceutically acceptable salt or solvate thereof (“active ingredient”)] and a pharmaceutically acceptable carrier, which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Formulations for parenteral administration include aqueous and non-aqueous sterile injection solutions which may contain anti-oxidants, buffers, bacteriostats and solutes which render the formulation isotonic with the blood of the intended recipient. Formulations for parenteral administration also include aqueous and non-aqueous sterile suspensions, which may include suspending agents and thickening agents. The formulations may be presented in unit-dose of multi-dose containers, for example sealed ampoules and vials, and may be stored in a freeze-dried (lyophilized) condition requiring only the addition of a sterile liquid carrier, for example saline, phosphate-buffered saline (PBS) or the like, immediately prior to use. Extemporaneous injection solutions and suspensions may be prepared from sterile powders, granules and tablets of the kind previously described.

Formulations for rectal administration may be presented as a suppository with the usual carriers, such as cocoa butter or polyethylene glycol.

Formulations for topical administration in the mouth, for example buccally or sublingually, include lozenges comprising the active ingredient in a flavored basis such as sucrose and acacia or tragacanth, and pastilles comprising the active ingredient in a basis such as gelatin and glycerin or sucrose and acacia.

Preferred unit dosage formulations are those containing an effective dose, or an appropriate fraction thereof, of the active ingredient.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, lubricating, surface active or dispersing agent. Molded tablets may be made by molding in a suitable machine a mixture of the powdered compound moistened with an inert liquid diluent. The tablets may optionally be coated or scored and may be formulated so as to provide sustained, delayed or controlled release of the active ingredient therein.

The compounds of formula I are preferably administered orally or by injection (intravenous or subcutaneous). The dose range for adult humans is generally from 0.005 mg to 10 g/day orally. Tablets or other forms of presentation provided in discrete units may conveniently contain an amount of compound of the invention which is effective at such dosage or as a multiple of the same, for instance, units containing 5 mg to 500 mg, usually around 10 mg to 200 mg. The precise amount of compound administered to a patient will be the responsibility of the attendant physician. However, the dose employed will depend on a number of factors, including the age and sex of the patient, the precise disorder being treated, and its severity. Also, the route of administration may vary depending on the condition and its severity.

As used herein, and as would be understood by the person of skill in the art, the recitation of “a compound” is intended to include salts, solvates and inclusion complexes of that compound. Thus, for example, the recitation “a compound” as depicted above would include salts in which —NR² is NH⁺Y⁻, wherein Y is any suitable counterion.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C— or ¹⁴C-enriched carbon are within the scope of this invention. Such compounds are useful, for example, as analytical tools or probes in biological assays.

The variables are defined when introduced and retain that definition throughout. Thus, for example, R² is always chosen from aryl, substituted aryl, arylalkyl, substituted arylalkyl, heteroaryl, substituted heteroaryl, hetroarylalkyl, and substituted heteroarylalkyl, although, according to standard patent practice, in dependent claims it may be restricted to a subset of these values. Superscripts are added to distinguish among residues that are attached similarly and that have overlapping Markush groups. For example, the substituent attached to the imidazolone ring at the nitrogen of the imidazolone can be R² or R^{2a} depending on the members of the Markush group defining it. For simplicity, the dependent claims, when multiply dependent, may refer to R² or R^{2a}. This is intended to modify the appropriate value of the corresponding variable R² or R^{2a} in each claim from which it depends. Thus a claim that recites “a compound according to any of claims 1 to 4 wherein R² is

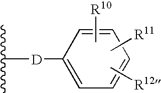

intends to further limit, for example, the corresponding R^{2a} substituent in claim 4.

The abbreviations Me, Et, Ph, Tf, Ts and Ms represent methyl, ethyl, phenyl, trifluoromethanesulfonyl, toluenesulfonyl and methanesulfonyl respectively. A comprehensive list of abbreviations utilized by organic chemists (i.e. persons of ordinary skill in the art) appears in the first issue of each volume of the Journal of Organic Chemistry. The list, which is typically presented in a table entitled “Standard List of Abbreviations,” is incorporated herein by reference.

### ABBREVIATIONS

The following abbreviations and terms have the indicated meanings throughout:
Ac=acetyl
anh.=anhydrous
APC=antigen presenting cells
atm=atmosphere
BNB=4-bromomethyl-3-nitrobenzoic acid
Boc=t-butyloxy carbonyl
Bu=butyl
BuOH=butanol
CBZ=carbobenzoxy=benzyloxycarbonyl
CDCl₃=deuterated chloroform
CDI=carbonyl diimidazole
DBU=diazabicyclo[5.4.0]undec-7-ene
DCM=dichloromethane=methylene chloride=CH₂Cl₂
DEAD=diethyl azodicarboxylate
DIC=diisopropylcarbodiimide
DIEA=N,N-diisopropylethyl amine
DMAP=4-N,N-dimethylaminopyridine
DMF=N,N-dimethylformamide
DMSO=dimethyl sulfoxide
DPPA=diphenylphosphoryl azide
DVB=1,4-divinylbenzene
EEDQ=2-ethoxy-1-ethoxycarbonyl-1,2-dihydroquinoline
Et=ethyl
EtOAc=ethyl acetate
FCC=flash column chromatography
Fmoc=9-fluorenylmethoxycarbonyl
GC=gas chromatography
HATU=O-(7-Azabenzotriazol-1-yl)-1,1,3,3-tetramethyluronium hexafluorophosphate
HOAc=acetic acid
HOBt=hydroxybenzotriazole
IMAP=Immobilized Metal Affinity Particle
Me=methyl
MeOH=methyl alcohol (methanol)
mesyl or Ms=methanesulfonyl
MTBE=methyl t-butyl ether
NBS=N-Bromosuccimide
NMO=N-methylmorpholine oxide
PEG=polyethylene glycol
PfP=pentafluorophenol
Ph=phenyl
PhOH=phenol
PhP₃=triphenylphosphine
PKC=protein kinase C
PPTS=pyridinium p-toluenesulfonate
PyBroP=bromo-tris-pyrrolidino-phosphonium hexafluorophosphate
rt=room temperature
sat'd=saturated
TBDMS=t-butyldimethylsilyl
TCR=T-cell receptor
Tf=trifluoromethanesulfonyl
TFA=trifluoroacetic acid
THF=tetrahydrofuran
TIPSO=triisopropylsilanyloxy
TLC=thin layer chromatography
TMOF=trimethyl orthoformate
TMS=trimethylsilyl
tosyl=p-toluenesulfonyl
Trt=triphenylmethyl
Ts=toluenesulfonyl

The data presented herein demonstrates utility of the compounds of the invention in inhibition of PKCθ and their utility for treatment of T-cell mediated diseases including autoimmune diseases such as rheumatoid arthritis, lupus erythematosus, and multiple sclerosis, inflammatory diseases such as asthma and inflammatory bowel disease, transplant rejection, gastrointestinal cancer, and diabetes.

Although this invention is susceptible to embodiment in many different forms, preferred embodiments of the invention are shown. It should be understood, however, that the present disclosure is to be considered as an exemplification of the principles of this invention and is not intended to limit the invention to the embodiments illustrated.

All of the compounds falling within the foregoing parent genus and its subgenera are useful as PKCθ inhibitors, but not all the compounds are novel. In particular, certain known species fall within the genus I, although no utility in inhibiting PKCθ has been suggested for these species. It may be found upon examination that compounds that have been excluded from the claims to compounds or compounds that have been excluded from the claims to methods are patentable to the inventors in this application; it may also be found that additional species and genera not presently excluded are not patentable to the inventors in this application. In either case, the exclusion of species and genera in applicants' claims are to be considered artifacts of patent prosecution and not reflective of the inventors' concept or description of their invention. The invention, in a composition aspect, is all compounds of formula I except those that are in the public's possession. In particular, a search of the literature indicates that 8H-Purin-8-one, 2-[[2-(2-fluorophenyl)ethyl]amino]-7,9-dihydro-9-(3-methoxyphenyl) and 8H-Purin-8-one, 9-[(2,6-difluorophenyl)methyl]-2-[[(2,4-dimethoxyphenyl)methyl]amino]-7,9-dihydro have been disclosed for other indications.

In general, the compounds of the present invention may be prepared by the methods illustrated in the general reaction schemes as, for example, described below, or by modifications thereof, using readily available starting materials, reagents and conventional synthesis procedures. In these reactions, it is also possible to make use of variants that are in themselves known, but are not mentioned here.

### Synthesis of Target Compounds

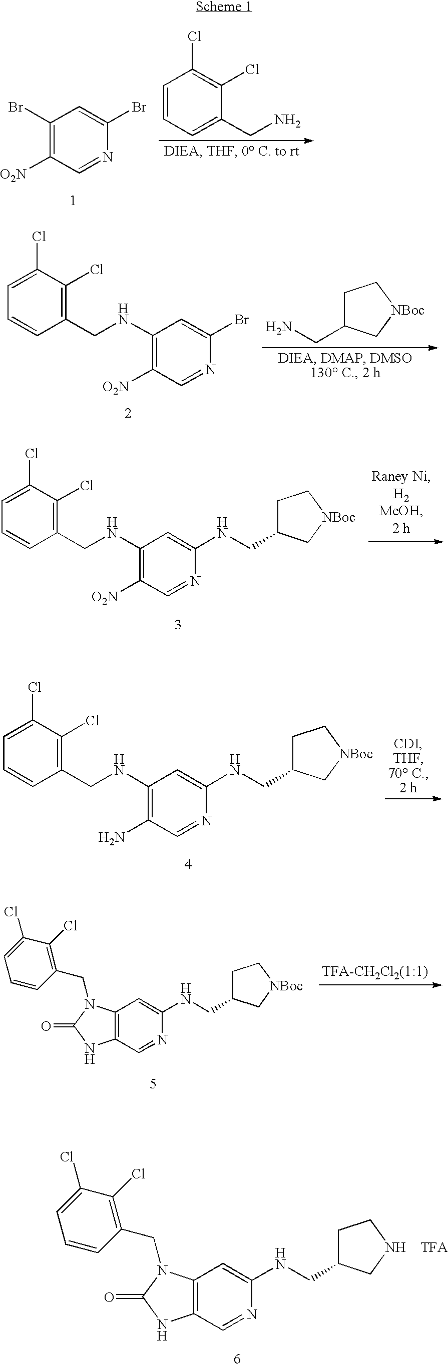

### 2,4-Dibromo-5-nitropyridine (1)

2,4-Dibromo-5-nitropyridine (1) was prepared in two steps from the commercially available 4-methoxy-3-nitropyridine according to the method reported by the literature.¹ LC-MS: Rt 6.23 min, m/e 282.7; ¹H NMR (CDCl₃) δ 8.82 (s, 1H), 7.96 (s, 1H).
1. Stavenger, R. A.; et al. J. Med. Chem. 2007, 50, 2.

### 2-Bromo-4-(2,3-dichlorobenzylamino)-5-nitropyridine (2)

To a solution of 2,4-dibromo-5-nitropyridine (1) (0.5 g, 1.77 mmol) in THF (10 mL) at 0° C. was added a mixture of 2,3-dichlorobenzylamine (0.32 g, 1.82 mmol), diisopropylethylamine (0.62 mL, 3.56 mmol), and THF (10 mL) dropwise with stirring under argon. After addition, the resulting mixture was stirred at room temperature for 5 h when TLC analysis (EtOAc-hexanes, 1:4) showed that the reaction was complete. The mixture was then poured into cold water (60 mL) and extracted with DCM (2×60 mL). The combined organic layers were washed with brine, dried over MgSO₄, and concentrated under reduced pressure. The residue was purified by column chromatography (EtOAc-hexanes, 1:6) to give 2 as a yellow solid (605 mg, 91%). LC-MS: Rt 7.72 min, m/e 378.0; ¹H NMR (CDCl₃) δ 9.00 (s, 1H), 8.57 (s, broad, 1H, NH), 7.48 (d, 1H), 7.23 (d, 1H), 7.18 (m, 1H), 6.82 (s, 1H), 4.61 (d, 2H).

### (S)-tert-Butyl 3-((4-(2,3-Dichlorobenzylamino)-5-nitropyridin-2-ylamino)methyl)pyrrolidine-1-carboxylate (3)

A mixture of 2 (150 mg, 0.398 mmol), (S)-tert-butyl 3-(aminomethyl)pyrrolidine-1-carboxylate (160 mg, 0.799 mmol), diisopropylethylamine (0.2 mL, 1.15 mmol), 4-(dimethylamino)pyridine (10 mg), and DMSO (7 ml) was stirred at 130° C. under argon for 2 h when LC-MS analysis showed the disappearance of the starting material. The mixture was then cooled to room temperature, and DCM (40 mL) and water (30 mL) were added. The organic layer was separated, dried over MgSO₄, and concentrated under reduced pressure. The residue was purified by column chromatography (DCM-MeOH, 25:1) to give 3 as a yellow solid (165 mg, 84%). LC-MS: Rt 6.61 min, m/e 440.1, 496.2; ¹H NMR (CDCl₃) δ 8.97 (s, 1H), 8.61 (s, broad, 1H, NH), 7.42 (d, 1H), 7.20 (m, 2H), 5.25 (s, 1H), 4.60 (d, 2H), 3.50-3.15 (m, 7H), 2.25 (m, 1H), 1.95 (m, 1H), 1.41 (s, 9H).

### (S)-tert-Butyl 3-((5-Amino-4-(2,3-dichlorobenzylamino)pyridin-2-ylamino)methyl)pyrrolidine-1-carboxylate (4)

A mixture of 3 (165 mg, 0.333 mmol), MeOH (15 mol), and Raney® 2800 Ni was subjected to hydrogenation using H₂ balloon at room temperature for 2 h when LC-MS analysis showed the disappearance of the starting material. The mixture was filtered through a layer of Celite and washed with MeOH. The filtrate and washings were then concentrated under reduced pressure and the residue was dried under vacuum for 4 h to give the crude amine 4, which was used in next cyclization step without purification. LC-MS: Rt 5.88 min, m/e 466.2, 468.2.

### (S)-tert-Butyl 3-((3-(2,3-Dichlorobenzyl)-2-oxo-2,3-dihydro-1H-imidazo[4,5-c]pyridin-5-ylamino)methyl)pyrrolidine-1-carboxylate (5)

A mixture of the crude amine 4 (ca. 0.333 mmol), THF (15 mL), and 1,1′-carbonyldiimidazole (194 mg, 1.2 mmol) was stirred at 70° C. under argon for 2 h when LC-MS analysis showed that the cyclization was complete. The mixture was then cooled to room temperature, and DCM (40 mL) and water (40 mL) were added. The organic layer was separated, dried over MgSO₄, and concentrated under reduced pressure. The residue was purified by column chromatography (DCM-MeOH, 20:1) to give 5 as an off-white solid (105 mg). LC-MS: Rt 5.45 min, m/e 392.2, 492.2; ¹H NMR (CDCl₃) δ 7.80 (s, 1H), 7.38 (d, 1H), 7.10 (t, 1H), 6.92 (d, 1H), 5.82 (s, 1H), 5.08 (s, 2H), 4.75 (m, 1H), 3.58-3.00 (m, 7H), 2.42 (m, 1H), 1.98 (m, 1H), 1.60 (m, 1H), 1.42 (s, 9H).

### (R)-3-(2,3-Dichlorobenzyl)-5-(pyrrolidin-3-ylmethylamino)-1H-imidazo[4,5-c]pyridine-2(3H)-one (6)

To a solution of 5 (105 mg) in DCM (2 mL) was added a solution of TFA (2 mL) in DCM (2 mL) dropwise with stirring at room temperature. The mixture was then stirred at room temperature for 1 h when LC-MS analysis showed that the deprotection was complete. The mixture was concentrated under reduced pressure. The resultant residue was dried under vacuum to give the desired TFA salt 6 as an off-white solid (104 mg). LC-MS: Rt 4.02 min, m/e 392.2; ¹H NMR (CD₃OD) δ 7.46 (s, 1H), 7.38 (d, 1H), 7.11 (t, 1H), 6.94 (d, 1H), 6.31 (s, 1H), 5.06 (s, 2H), 3.38-3.05 (m, 5H), 2.81 (m, 1H), 2.50 (m, 1H), 2.08 (m, 1H), 1.61 (m, 1H).

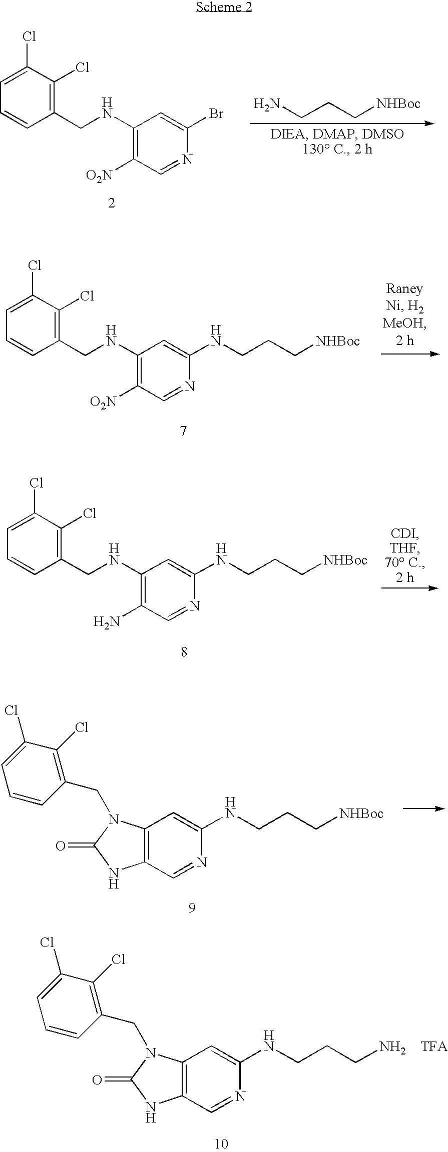

### tert-Butyl 3-(4-(2,3-Dichlorobenzylamino)-5-nitropyridin-2-ylamino)propyl carbamate (7)

The intermediate 7 was prepared from 2 and tert-butyl (3-aminopropyl)carbamate in a similar method as described for the intermediate 3. LC-MS: Rt 6.15 min, m/e 414.2, 470.2; ¹H NMR (CDCl₃) δ 8.97 (s, 1H), 8.61 (s, broad, 1H, NH), 7.42 (d, 1H), 7.20 (m, 2H), 5.25 (s, 1H), 4.61 (d, 2H), 3.35 (m, 2H), 3.11 (m, 2H), 1.82 (m, 2H), 1.41 (s, 9H).

### tert-Butyl 3-(5-Amino-4-(2,3-dichlorobenzylamino)pyridin-2-ylamino)propyl carbamate (8)

The crude intermediate 8 was prepared from 7 in a similar method as described for the intermediate 4. LC-MS: Rt 5.70 min, m/e 440.2, 443.3.

### tert-Butyl 3-(3-(2,3-Dichlorobenzyl)-2-oxo-2,3-dihydro-1H-imidazo[4,5-c]pyridin-5-ylamino)propylcarbamate (9)

The intermediate 9 was prepared from 8 in a similar method as described for the intermediate 5. LC-MS: Rt 5.29 min, m/e 466.2, 469.3; ¹H NMR (CDCl₃) δ 7.81 (s, 1H), 7.39 (d, 1H), 7.10 (t, 1H), 6.95 (d, 1H), 5.83 (s, 1H), 5.12 (s, 2H), 3.25 (m, 2H), 3.11 (m, 2H), 1.70 (m, 2H), 1.42 (s, 9H).

### 3-(2,3-Dichlorobenzyl)-5-(3-aminopropylamino)-1H-imidazo[4,5-c]pyridine-2(3H)-one (10)

The target compound 10 was prepared from 9 in a similar method as described for the compound 6. LC-MS: Rt 3.90 min, m/e 366.1; ¹H NMR (CD₃OD) δ 7.57 (s, 1H), 7.41 (d, 1H), 7.10 (t, 1H), 7.02 (d, 1H), 6.38 (s, 1H), 5.10 (s, 2H), 3.38 (t, 2H), 2.92 (t, 2H), 1.92 (m, 2H).

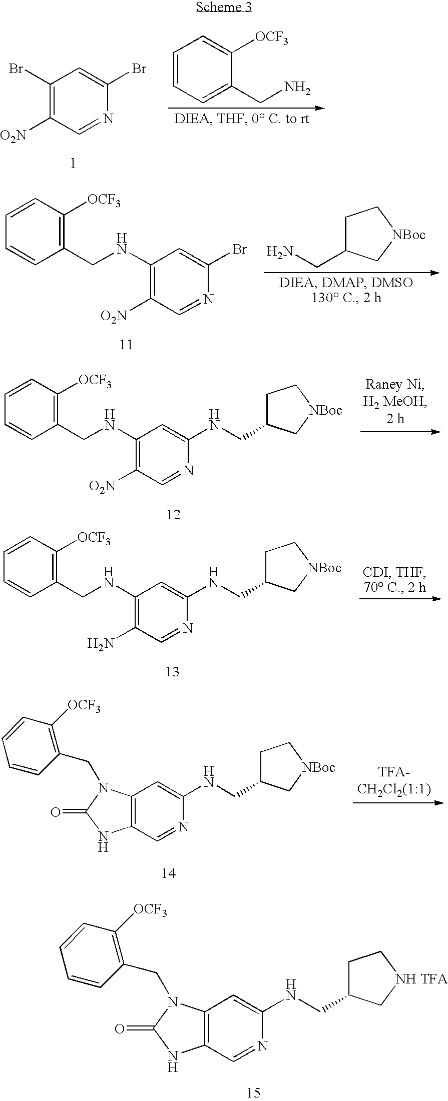

### 2-Bromo-4-(2-(trifluoromethoxy)benzylamino)-5-nitropyridine (11)

The intermediate 11 as a yellow solid was prepared from 1 and 2-(trifluoromethoxy)benzylamine in a similar method as described for the intermediate 2. LC-MS: Rt 7.47 min, m/e 394.1, 395.2; ¹H NMR (CDCl₃) δ 9.00 (s, 1H), 8.52 (s, broad, 1H, NH), 7.42-7.28 (m, 4H), 6.88 (s, 1H), 4.61 (d, 2H).

### (S)-tert-Butyl 3-((5-Nitro-4-(2-(trifluoromethoxy)benzylamino)pyridin-2-ylamino)methyl)pyrrolidine-1-carboxylate (12)

The intermediate 12 was prepared from 11 and (S)-tert-butyl 3-(aminomethyl)pyrrolidine-1-carboxylate in a similar method as described for the intermediate 3. LC-MS: Rt 6.36 min, m/e 512.3; ¹H NMR (CDCl₃) δ 8.97 (s, 1H), 8.59 (s, broad, 1H, NH), 7.40-7.28 (m, 4H), 5.22 (s, 1H), 4.59 (d, 2H), 3.50-3.10 (m, 6H), 3.09-2.95 (m, 1H), 2.29 (m, 1H), 1.95 (m, 1H), 1.41 (s, 9H).

### (S)-tert-Butyl 3-((5-Amino-4-(2-(trifluoromethoxy)benzylamino)pyridin-2-ylamino)methyl)pyrrolidine-1-carboxylate (13)

The crude intermediate 13 was prepared from 12 in a similar method as described for the intermediate 4. LC-MS: Rt 5.59 min, m/e 482.3, 483.4.

### (S)-tert-Butyl 3-((3-(2-(Trifluoromethoxy)benzyl)-2-oxo-2,3-dihydro-1H-imidazo[4,5-c]pyridin-5-ylamino)methyl)pyrrolidine-1-carboxylate (14)

The intermediate 14 was prepared from 13 in a similar method as described for the intermediate 5. LC-MS: Rt 5.42 min, m/e 508.3; ¹H NMR (CDCl₃) δ 7.81 (s, 1H), 7.38-7.22 (m, 4H), 5.86 (s, 1H) 5.08 (s, 2H), 4.55 (m, 1H), 3.58-3.00 (m, 6H), 2.42 (m, 1H), 1.98 (m, 1H), 1.60 (m, 1H), 1.42 (s, 9H).

### (R)-3-(2-(Trifluoromethoxy)benzyl)-5-(pyrrolidin-3-ylmethylamino)-1H-imidazo[4,5-c]pyridine-2(3H)-one (15)

The target compound 15 was prepared from 14 in a similar method as described for the compound 6. LC-MS: Rt 3.98 min, m/e 408.3, 409.4; ¹H NMR (CD₃OD) δ 7.57 (s, 1H), 7.40 (d, 1H), 7.34-7.27 (m, 3H), 6.37 (s, 1H), 5.08 (s, 2H), 3.42-3.08 (m, 5H), 2.91 (m, 1H), 2.60 (m, 1H), 2.19 (m, 1H), 1.71 (m, 1H).

### Synthesis of Target Compounds

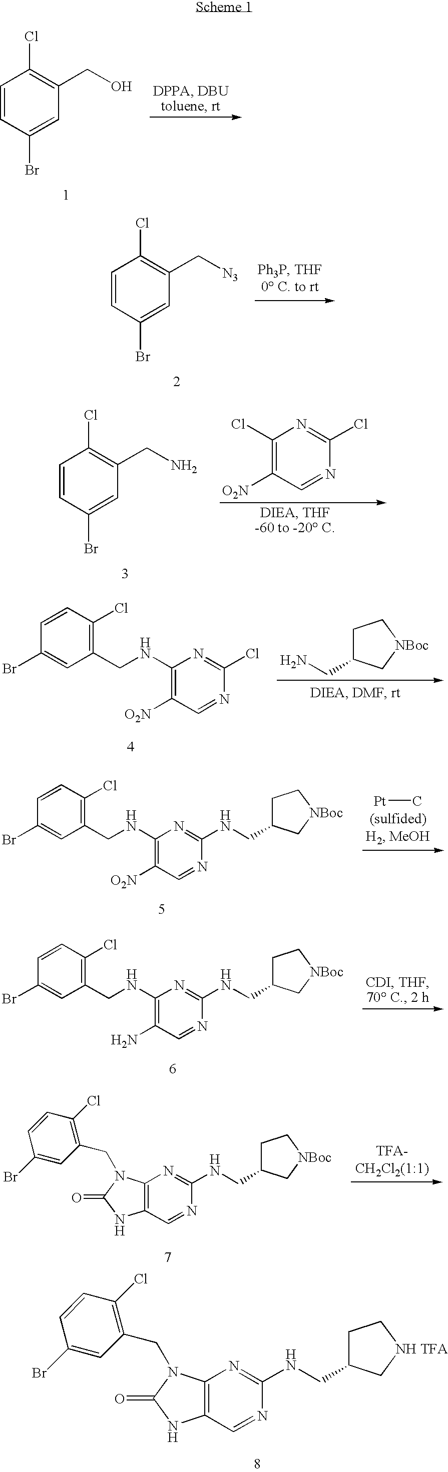

### 5-Bromo-2-chlorobenzyl Azide (2)

To a mixture of 5-bromo-2-chlorobenzyl alcohol (2.5 g, 11.31 mmol), toluene (40 mL), DPPA (2.8 mL, 12.95 mmol, 1.15 eq) at room temperature was added DBU (2 mL, 13.37 mmol, 1.18 eq) dropwise with stirring under argon. The mixture was then stirred at room temperature for 3 h when TLC analysis (EtOAc-hexanes, 1:4) showed that the starting material had been consumed. EtOAc (150 mL) and water (100 mL) were added, and the organic layer was separated, washed with 0.5N HCl solution, dried over MgSO₄, and concentrated under reduced pressure. The colorless liquid residue was purified by column chromatography (EtOAc-hexanes, 1:20) to give 2 as a colorless liquid (2.4 g, 86% yield). R_{f} 0.67 (EtOAc-hexanes, 1; 20); ¹H NMR (CDCl₃) δ 7.58 (s, 1H), 7.41 (d, 1H), 7.24 (d, 1H), 4.46 (s, 2H).

### 5-Bromo-2-chlorobenzylamine (3)

To a solution of the azide 2 (2.4 g, 9.76 mmol) in THF (60 mL) at 0° C. was added triphenylphosphine (2.86 g, 10.90 mmol, 1.12 eq) slowly with stirring. After addition, the mixture was stirred at 0° C. for 10 min and at room temperature overnight. TLC analysis (EtOAc-hexanes, 1:20) showed that the starting material disappeared. Ammonium hydroxide solution (10 mL) was added dropwise. After stirring for 30 min, 1N NaOH solution (20 mL) was added, the resulting mixture was stirred at room temperature for 2 h. Ether (100 mL) and water (80 mL) were added. The mixture was made acidic (pH 2) by adding 4N H₂SO₄ solution slowly. The aqueous layer was then separated, made alkaline by adding 4N NaOH solution, and extracted with DCM (2×100 mL). The combined organic layers were dried over MgSO₄ and concentrated under reduced pressure. The residue was purified by column chromatography (MeOH-DCM, 1:20) to give 3 as a colorless liquid (1.45 g, 67% yield). LC-MS: Rₜ 3.67 min, m/e 221.1, 205.1; ¹H NMR (CDCl₃) δ 7.64 (s, 1H), 7.40 (dd, 1H), 7.28 (d, 1H), 4.02 (s, 2H), 1.58 (s, broad, 2H, NH₂).

### 4-(5-Bromo-2-chlorobenzylamino)-2-chloro-5-nitropyrimidine (4)

To a solution of 2,4-dichloro-5-nitropyrimidine (1.13 g, 5.82 mmol) in THF (20 mL) at −60° C. was added a mixture of 5-bromo-2-chlorobenzylamine (3) (1.28 g, 5.82 mmol, 1 eq), diisopropylethylamine (2.03 mL, 11.65 mmol, 2 eq), and THF (10 mL) dropwise with stirring under argon. After addition, the resulting mixture was stirred at −60° C. for 30 min and allowed to warm to −20° C. over a period of 1 h when TLC analysis (EtOAc-hexanes, 1:4) and LC-MS analysis showed that the reaction was complete. The mixture was then poured into cold water (60 mL) and extracted with DCM (2×80 mL). The combined organic layers were washed with brine (60 mL), dried over MgSO₄, and concentrated under reduced pressure. The residue was purified by column chromatography (EtOAc-hexanes, 1:5) to give 4 as a white solid (1.89 g, 86% yield). LC-MS: Rₜ 7.73 min, m/e 379.1, 377.0; ¹H NMR (CDCl₃) δ 9.03 (s, 1H), 8.78 (s, broad, 1H, NH), 7.60 (s, 1H), 7.41 (d, 1H), 7.23 (d, 1H), 4.85 (d, 2H).

### (S)-tert-Butyl 3-((4-(5-Bromo-2-chlorobenzylamino)-5-nitropyrimidin-2-ylamino)methyl)pyrrolidine-1-carboxylate (5)

A mixture of 4 (1.2 g, 3.17 mmol), (S)-tert-butyl 3-(aminomethyl)pyrrolidine-1-carboxylate (763 mg, 3.81 mmol, 1.2 eq), diisopropylethylamine (1.3 mL, 7.46 mmol, 2.4 eq), and DMF (20 mL) was stirred at room temperature under argon overnight when TLC analysis (EtOAc-hexanes, 1:5) and LC-MS analysis showed the disappearance of the starting material. DCM (100 mL) and water (100 mL) were added, and the organic layer was separated, washed with water (60 mL), dried over MgSO₄, and concentrated under reduced pressure. The residue was purified by column chromatography (EtOAc-hexanes, 1:2) to give 5 as a pale yellow solid (1.51 g, 88% yield). LC-MS: Rₜ 7.89 min, m/e 541.2, 487.1, 443.2; ¹H NMR (CDCl₃) δ 8.95 (s, 1H), 7.43 (s, 1H), 7.30 (d, 1H), 7.21 (d, 1H), 4.78 (d, 2H), 3.52-3.11 (m, 6H), 3.01 (m, 1H), 2.34 (m, 1H), 1.91 (m, 1H), 1.41 (s, 9H).

### (S)-tert-Butyl 3-((5-Amino-4-(5-bromo-2-chlorobenzylamino)pyrimidin-2-ylamino)methyl)pyrrolidine-1-carboxylate (6)

A mixture of 5 (500 mg, 0.924 mmol), MeOH (15 mL), and 5% Pt—C (sulfided) (80 mg) was subjected to hydrogenation using H₂ balloon (1 ATM) at room temperature for 4 h, when LC-MS analysis showed the disappearance of the starting material. The mixture was filtered through a layer of Celite and washed with MeOH. The filtrate and washings were then concentrated under reduced pressure and the residue was dried under vacuum for 4 h to give the crude amine 6, which was used in the next cyclization step without purification. LC-MS: Rₜ 5.82 min, m/e 515.0, 513.2.

### (S)-tert-Butyl 3-((9-(5-Bromo-2-chlorobenzyl)-8-oxo-8,9-dihydro-7H-purin-2-ylamino)methyl)pyrrolidine-1-carboxylate (7)

A mixture of the crude amine 6 (ca. 0.924 mmol), THF (25 mL), and 1,1′-carbonyldiimidazole (450 mg, 2.78 mmol, 3 eq) was stirred at 70° C. under argon for 2 h when LC-MS analysis showed that the cyclization was complete. The mixture was then cooled to room temperature, and DCM (100 mL) and water (60 mL) were added. The organic layer was separated, washed with water (60 mL), dried over MgSO₄, and concentrated under reduced pressure. The residue was purified by column chromatography (DCM-MeOH, 20:1) to give 7 as an off-white solid (451 mg, 90% yield from 5). LC-MS: Rₜ 5.46 min, m/e 539.3, 483.1, 481.1; ¹H NMR (CDCl₃) δ 9.62 (s, broad, 1H, NH), 7.83 (s, 1H), 7.38 (m, 2H), 7.21 (d, 1H), 5.08 (s, 2H), 3.75 (m, 1H), 3.58-3.10 (m, 5H), 3.05 (m, 1H), 2.42 (m, 1H), 1.92 (m, 1H), 1.60 (m, 1H), 1.42 (s, 9H).

### (R)-9-(5-Bromo-2-chlorobenzyl)-2-(pyrrolidin-3-ylmethylamino)-7H-purin-8(9H)-one (8)

To a solution of 7 (47 mg) in DCM (2 mL) was added a solution of TFA (2 mL) in DCM (2 mL) dropwise with stirring at room temperature. The mixture was then stirred at room temperature for 1 h when LC-MS analysis showed that the deprotection was complete. The mixture was concentrated under reduced pressure. The resultant residue was dried under vacuum to give the desired TFA salt 8 as a white solid (50 mg). LC-MS: Rₜ 3.98 min, m/e 439.2; ¹H NMR (CD₃OD) δ 7.70 (s, 1H), 7.49 (s, 1H), 7.40 (d, 1H), 7.25 (d, 1H), 5.06 (s, 2H), 3.40-3.09 (m, 5H), 2.92 (m, 1H), 2.54 (m, 1H), 2.10 (m, 1H), 1.64 (m, 1H).

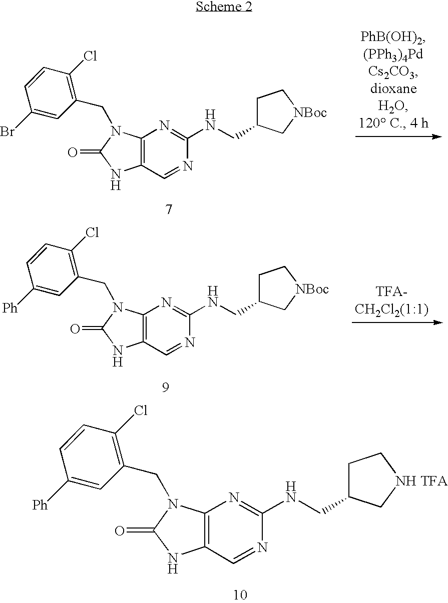

### (S)-tert-Butyl 3-((9-(2-Chloro-5-phenylbenzyl)-8-oxo-8,9-dihydro-7H-purin-2-ylamino)methyl)pyrrolidine-1-carboxylate (9)

A mixture of 7 (70 mg, 0.13 mmol), dioxane (6 mL), phenylboronic acid (24 mg, 0.20 mmol, 1.5 eq), cesium carbonate (169 mg, 0.519 mmol, 4 eq), tetrakis(triphenylphosphine)palladium(0) (15 mg, 0.0128 mmol, 0.1 eq), and water (2 mL) was stirred at 120° C. under argon for 4 h when LC-MS analysis showed that the reaction was complete. The mixture was then cooled to room temperature and filtered through a layer of Celite. To the filtrate were added DCM (60 mL) and water (40 mL). The organic layer was separated, dried over MgSO₄, and concentrated under reduced pressure. The residue was purified by column chromatography (DCM-MeOH, 20:1) to give 9 as an off-white solid (27 mg, 39% yield). LC-MS: Rₜ 5.93 min, m/e 535.3, 479.2; ¹H NMR (CDCl₃) δ 9.35 (s, broad, 1H, NH), 7.83 (s, 1H), 7.42-7.25 (m, 8H), 5.18 (s, 2H), 3.58-3.10 (m, 5H), 3.01 (m, 1H), 2.37 (m, 1H), 1.93-1.80 (m, 1H), 1.60 (m, 1H), 1.41 (s, 9H).

### (R)-9-(2-Chloro-5-phenylbenzyl)-2-(pyrrolidin-3-ylmethylamino)-7H-purin-8(9H)-one (10)

Using the deprotection method described above for the transformation of 7→8, the target compound 10 was isolated as the TFA salt (a yellow film) from its N-Boc-protected precursor 9. LC-MS: Rₜ 4.45 min, m/e 435.3; ¹H NMR (CD₃OD) δ 7.77 (s, 1H), 7.68-7.31 (m, 8H), 5.12 (s, 2H), 3.50-3.09 (m, 5H), 2.84 (m, 1H), 2.54 (m, 1H), 2.05 (m, 1H), 1.60 (m, 1H).

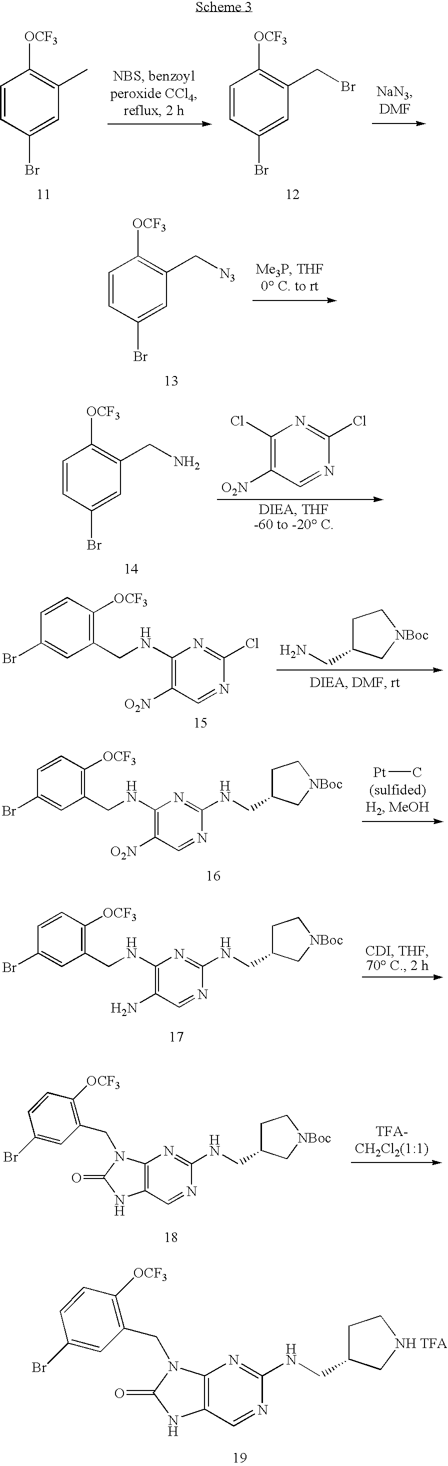

### 5-Bromo-2-(trifluoromethoxy)benzyl Bromide (12)

To a solution of 5-Bromo-2-(trifluoromethoxy)toluene (2 g, 7.84 mmol) in Carbon tetrachloride (70 mL) were added N-Bromosuccinimide (1.4 g, 7.86 mmol) and Benzoyl peroxide (100 mg) in turn. The mixture was then heated at reflux under argon with stirring for 2 h when TLC analysis (hexanes only) showed that the starting material had been consumed. The mixture was cooled to room temperature and filtered. The filtrate was concentrated under reduced pressure. The residue was purified by column chromatography (hexanes only) to give 12 as a colorless liquid (2.3 g, 88% yield). R_{f}0.63 (hexanes only); ¹H NMR (CDCl₃) δ 7.61 (s, 1H), 7.43 (d, 1H), 7.12 (d, 1H), 4.41 (s, 2H).

### 5-Bromo-2-(trifluoromethoxy)benzyl Azide (13)

A mixture of the Bromide 12 (2.3 g, 6.89 mmol), DMF (30 mL), and Sodium azide (0.76 g, 11.69 mmol, 1.7 eq) was stirred at room temperature overnight when TLC analysis (hexanes only) showed that the starting material had been consumed. EtOAc (120 mL) and water (80 mL) were added, and the organic layer was separated, washed with brine (60 mL), dried over MgSO₄, and concentrated under reduced pressure. The colorless liquid residue was purified by column chromatography (hexanes only) to give 13 as a colorless liquid (2.01 g, 99% yield). R_{f} 0.50 (hexanes only); ¹H NMR (CDCl₃) δ 7.59 (s, 1H), 7.44 (d, 1H), 7.15 (d, 1H), 4.41 (s, 2H); ¹⁹F NMR (CDCl₃) δ −57.8.

### 5-Bromo-2-(trifluoromethoxy)benzylamine (14)

To a solution of the Azide 13 (2 g, 6.76 mmol) in THF (35 mL) at 0° C. was added a solution of trimethylphosphine in THF (1M, 8.1 mL, 8.1 mmol, 1.2 eq) dropwise with stirring. After addition, the mixture was stirred at room temperature overnight when TLC analysis (hexanes only) showed that the starting material had been consumed. 1N NaOH solution (10 mL) was added, and the resulting mixture was stirred at room temperature for 1 h. Diethyl ether (100 mL) and water (80 mL) were added. The mixture was made acidic (pH 2) by adding 2.5NH₂SO₄ solution slowly. The aqueous layer was then separated, made alkaline by adding 4N NaOH solution, and extracted with Diethyl ether (2×100 mL). The combined organic layers were dried over MgSO₄ and concentrated under reduced pressure to give the crude amine 14 as a yellow liquid (1.2 g, 66% yield). LC-MS: Rₜ 4.29 min, m/e 255.0; ¹H NMR (CDCl₃) δ 7.61 (s, 1H), 7.38 (d, 1H), 7.08 (d, 1H), 3.87 (s, 2H); ¹⁹F NMR (CDCl₃) δ −58.2.

### 4-(5-Bromo-2-(trifluoromethoxy)benzylamino)-2-chloro-5-nitropyrimidine (15)

2,4-Dichloro-5-nitropyrimidine and 5-Bromo-2-(trifluoromethoxy)benzylamine (14) were subjected to reaction conditions, work up and silica gel chromatography similar to those described above for the transformation of 3→4. Intermediate 15 was thereby isolated as a pale yellow liquid in 88% yield. LC-MS: Rₜ8.04 min, m/e 429.1; ¹H NMR (CDCl₃) δ 9.02 (s, 1H), 8.62 (s, broad, 1H, NH), 7.61 (s, 1H), 7.42 (d, 1H), 7.18 (d, 1H), 4.89 (d, 2H); ¹⁹F NMR (CDCl₃) δ −57.6.

### (S)-tert-Butyl 3-((4-(5-Bromo-2-(trifluoromethoxy)benzylamino)-5-nitropyrimidin-2-ylamino)methyl)pyrrolidine-1-carboxylate (16)

Intermediate 15 and (S)-tert-Butyl 3-(aminomethyl)pyrrolidine-1-carboxylate were subjected to reaction conditions, work up and silica gel chromatography similar to those described above for the transformation of 4→5. Intermediate 16 was thereby isolated as a white solid in 75% yield. LC-MS: Rₜ 8.26 min, m/e 591.2, 537.1, 491.1; ¹H NMR (CDCl₃) δ 8.83 (s, 1H), 7.45 (s, 1H), 7.39 (d, 1H), 7.09 (d, 1H), 4.77 (d, 2H), 3.45-3.10 (m, 6H), 3.02-2.85 (m, 1H), 2.29 (m, 1H), 1.82 (m, 1H), 1.40 (s, 9H); ¹⁹F NMR (CDCl₃) δ −58.1.

### (S)-tert-Butyl 3-((5-Amino-4-(5-bromo-2-(trifluoromethoxy)benzylamino)pyrimidin-2-ylamino)methyl)pyrrolidine-1-carboxylate (17)

Intermediate 16 was subjected to reaction conditions and work-up similar to those described above for the transformation of 5→6. Crude Intermediate 17 was thereby isolated and used in the subsequent cyclization step without purification. LC-MS: Rₜ 5.73 min, m/e 564.3, 563.3.

### (S)-tert-Butyl 3-((9-(5-Bromo-2-(trifluoromethoxy)benzyl)-8-oxo-8,9-dihydro-7H-purin-2-ylamino)methyl)pyrrolidine-1-carboxylate (18)

Intermediate 17 was subjected to reaction conditions and work-up similar to those described above for the transformation of 6→7. Intermediate 18 was thereby isolated as a white solid. LC-MS: Rₜ 5.73 min, m/e 589.3. 533.1, 489.2; ¹H NMR (CDCl₃) δ 8.98 (s, broad, 1H, NH), 7.86 (s, 1H), 7.42 (m, 2H), 7.13 (d, 1H), 5.02 (s, 2H), 3.58-3.10 (m, 6H), 3.08 (m, 1H), 2.45 (m, 1H), 1.98 (m, 1H), 1.42 (s, 9H); ¹⁹F NMR (CDCl₃) δ −57.8.

### (R)-9-(5-Bromo-2-(trifluoromethoxy)benzyl)-2-(pyrrolidin-3-ylmethylamino)-7H-purin-8(9H)-one (19)

Using the deprotection method described above for the transformation of 7→8, the target compound 19 was isolated as the TFA salt from its N-Boc-protected precursor, Intermediate 18. LC-MS: Rₜ 4.16 min, m/e 489.2, 487.1; ¹H NMR (CD₃OD) δ 7.70 (s, 1H), 7.62 (s, 1H), 7.52 (d, 1H), 7.22 (d, 1H), 5.08 (s, 2H), 3.55-3.08 (m, 5H), 2.93 (m, 1H), 2.62 (m, 1H), 2.16 (m, 1H), 1.73 (m, 1H); ¹⁹F NMR (CDCl₃) δ −59.1, −77.5.

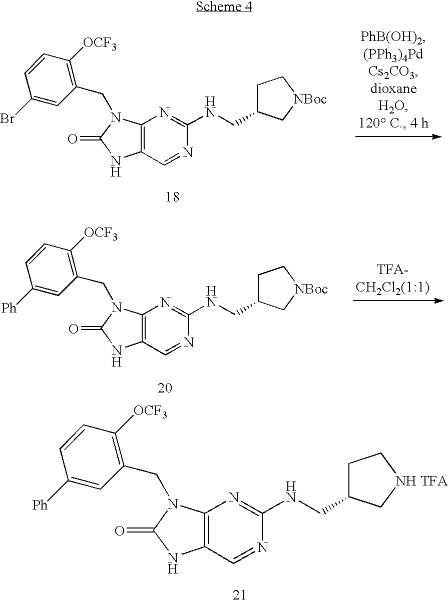

### (S)-tert-Butyl 3-((9-(5-Phenyl-2-(trifluoromethoxy)benzyl)-8-oxo-8,9-dihydro-7H-purin-2-ylamino)methyl)pyrrolidine-1-carboxylate (20)

Intermediate 18 was subjected to reaction conditions, work-up and silica gel chromatography similar to those described above for the transformation of 7→9. Intermediate 20 was thereby isolated as an off-white solid in 64% yield. LC-MS: Rₜ 6.15 min, m/e 585.4, 529.3; ¹H NMR (CDCl₃) δ 9.97 (s, broad, 1H, NH), 7.82 (s, 1H), 7.57-7.25 (m, 8H), 5.18 (s, 2H), 3.58-3.10 (m, 5H), 3.01 (m, 1H), 2.40 (m, 1H), 1.90 (m, 1H), 1.60 (m, 1H), 1.41 (s, 9H); ¹⁹F NMR (CDCl₃) δ −57.0.

### (R)-9-(5-Phenyl-2-(trifluoromethoxy)benzyl)-2-(pyrrolidin-3-ylmethylamino)-7H-purin-8(9H)-one (21)

Using the deprotection method described above for the transformation of 7→8, the target compound 21 was isolated as the TFA salt (pale yellow solid) from its N-Boc-protected precursor, Intermediate 20. LC-MS: Rₜ 4.61 min, m/e 485.3; ¹H NMR (CD₃OD) δ 7.76 (s, 1H), 7.67 (s, 1H), 7.60 (d, 1H), 7.53 (d, 2H), 7.38-7.22 (m, 4H), 5.15 (s, 2H), 3.60-3.02 (m, 5H), 2.84 (m, 1H), 2.52 (m, 1H), 1.92 (m, 1H), 1.51 (m, 1H); ¹⁹F NMR (CDCl₃) δ −59.2, −77.7.

### Preparation of (R)-9-(3-Iodobenzyl)-2-(pyrrolidin-3-ylmethylamino)-7H-purin-8(9H)-one TFA salt

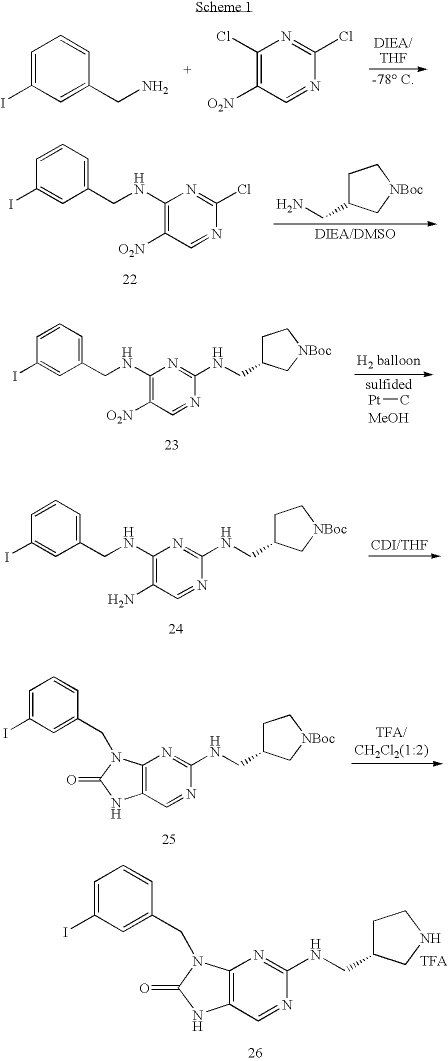

### Step 1 N-(3-Iodobenzyl)-2-chloro-5-nitropyrimidin-4-amine (22)

To a solution of 2,4-Dichloro-5-nitropyrimidine (2.34 g, 12.0 mmol, 1.0 eq) in THF (25 mL) at −78° C. was added Diisopropylethylamine (2.35 g, 18.0 mmol, 1.5 eq), followed by 3-Iodobenzylamine (2.80 g, 12.0 mmol, 1.0 eq). The mixture was stirred for 2 h, at which time TLC analysis showed complete consumption of starting material. The mixture was warmed to room temperature, diluted with brine and EtOAc, shaken and separated; the organic layer was washed with brine, dried on MgSO₄ and concentrated in vacuo. The resulting slush was purified on silica gel using 20% EtOAc in hexanes as eluent, yielding N-(3-Iodobenzyl)-2-chloro-5-nitropyrimidin-4-amine (4.40 g, 94%; Intermediate 22) as a pale yellow solid. ¹HNMR (CDCl₃, 300 MHz): δ 9.05 (s, 1H), 8.65 (brs, 1H), 7.70 (s, 1H), 7.65 (d, 1H), 7.35 (d, 1H), 7.15 (t, 1H), 4.80 (d, 2H).

### Step 2 (S)-tert-Butyl 3-((4-(3-iodobenzylamino)-5-nitropyrimidin-2-ylamino)methyl)pyrrolidine-1-carboxylate (23)

To a solution of N-(3-Iodobenzyl)-2-chloro-5-nitropyrimidin-4-amine (2.90 g, 7.4 mmol, 1.0 eq; Intermediate 22) in DMSO (12 ml) was added Diisopropylethylamine (1.43 g, 11.1 mmol, 1.5 eq), followed by (S)-tert-Butyl 3-(aminomethyl)pyrrolidine-1-carboxylate (1.55 g, 7.7 mmol, 1.04 eq) in DMSO (3 ml). The mixture was stirred at room temperature for 4 h when TLC analysis showed consumption of the starting material. The mixture was diluted with brine (50 ml), extracted with EtOAc (50 ml×3), dried on MgSO₄ and concentrated under reduced pressure. The residue was purified on silica gel using 20% EtOAc in hexanes as eluent, affording (S)-tert-Butyl 3-((4-(3-iodobenzylamino)-5-nitropyrimidin-2-ylamino)methyl)pyrrolidine-1-carboxylate (3.70 g, 90%; Intermediate 23) as a fluffy yellow solid. ¹HNMR (CDCl₃, 300 MHz): δ 8.95 (s, 1H), 8.85 (s, 1H), 7.65 (s, 1H), 7.60 (d, 1H), 7.25 (d, 1H), 7.05 (t, 1H), 5.80 (s, 1H), 4.70 (d, 2H), 3.60-3.20 (m, 6H), 3.00 (m, 1H), 2.40 (m, 1H), 1.95 (m, 1H), 1.45 (s, 9H).

### Step 3 (S)-tert-Butyl 3-((4-(3-iodobenzylamino)-5-aminopyrimidin-2-ylamino)methyl)pyrrolidine-1-carboxylate (24)

To a solution of (S)-tert-Butyl 3-((4-(3-iodobenzylamino)-5-nitropyrimidin-2-ylamino)methyl)pyrrolidine-1-carboxylate (0.50 g, 0.90 mmol; Intermediate 23) in MeOH (15 ml) was added 5% sulfided Pt/C (150 mg). The mixture was stirred under hydrogen balloon (1 ATM) for 5 h when MS analysis showed complete consumption of starting material and the formation of a peak consistent with the molecular weight of the desired product. The reaction mixture was filtered through celite and the filtrate was concentrated in vacuo to afford crude (S)-tert-Butyl 3-((4-(3-iodobenzylamino)-5-aminopyrimidin-2-ylamino)methyl)pyrrolidine-1-carboxylate (0.37 g, 79%; Intermediate 24) as an amber slush. This material was used directly in the subsequent cyclization step without purification.

### Step 4 (S)-tert-Butyl 3-((9-(3-iodobenzyl)-8-oxo-8,9-dihydro-7H-purin-2-ylamino)methyl)pyrrolidine-1-carboxylate (25)

Crude Intermediate 24 from the previous step (0.37 g, 0.71 mmol) was dissolved in THF (4 ml) and treated with 1,1′-carbonyldiimidazole (0.28 g, 1.73 mmol, 2.4 eq). The mixture was stirred at room temperature overnight. The reaction was concentrated to dryness in vacuo. The resulting residue was taken up in DCM and water, shaken, separated, and concentrated by rotoevaporation. The crude residue was applied to a silica gel column and eluted using 10% MeOH in DCM as eluent to give (S)-tert-Butyl 3-((9-(3-iodobenzyl)-8-oxo-8,9-dihydro-7H-purin-2-ylamino)methyl)pyrrolidine-1-carboxylate (0.15 g, 38%; Intermediate 25) as a pale purple solid. ¹HNMR (CDCl₃, 300 MHz): δ 8.50 (brs, 1H), 7.85 (s, 1H), 7.80 (s, 1H), 7.60 (t, 1H), 7.40 (d, 1H), 7.05 (t, 1H), 5.40 (brs, 1H), 4.95 (s, 2H), 3.60-3.40 (m, 3H), 3.35 (m, 1H), 3.15 (m, 1H), 2.75 (m, 1H), 2.55 (m, 1H), 2.00 (m, 1H), 1.90 (m, 1H), 1.45 (s, 9H).

### Step 5 (R)-9-(3-iodobenzyl)-2-(pyrrolidin-3-ylmethylamino)-7H-purin-8(9H)-one TFA salt (26)

(S)-tert-Butyl 3-((9-(3-iodobenzyl)-8-oxo-8,9-dihydro-7H-purin-2-ylamino)methyl)pyrrolidine-1-carboxylate (67 mg, 0.12 mmol; Intermediate 25) was stirred in a mixture of DCM/TFA (2:1; 3 mL) for 2 h at room temperature. The reaction was concentrated in vacuo to give (R)-9-(3-Iodobenzyl)-2-(pyrrolidin-3-ylmethylamino)-7H-purin-8(9H)-one [68 mg, quantitative; 26] as an amber foam TFA salt. ¹HNMR (CD₃OD, 300 MHz): δ 7.85 (s, 1H), 7.80 (s, 1H), 7.65 (t, 1H), 7.40 (t, 1H), 7.15 (t, 1H), 5.05 (s, 2H), 3.60 (m, 1H), 3.60-3.30 (m, 3H), 3.25 (m, 1H), 3.10 (m, 1H), 2.80 (m, 1H), 2.20 (m, 1H), 1.80 (m, 1H). LCMS: 6.7 min, M⁺=451.2.

### Preparation of (R)-9-(3-(2-Fluorophenyl)benzyl)-2-(pyrrolidin-3-ylmethylamino)-7H-purin-8(9H)-one TFA salt (28)

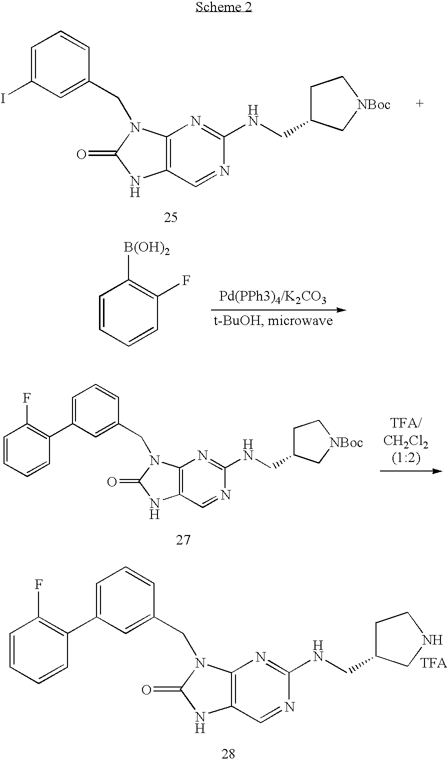

### Step 1 (S)-tert-Butyl 3-((9-(3-(2-fluorophenyl)benzyl)-8-oxo-8,9-dihydro-7H-purin-2-ylamino)methyl)pyrrolidine-1-carboxylate (27)

A mixture of (S)-tert-Butyl 3-((9-(3-iodobenzyl)-8-oxo-8,9-dihydro-7H-purin-2-ylamino)methyl)pyrrolidine-1-carboxylate (50 mg, 0.09 mmol, 1.0 eq; Intermediate 25) and 2-Fluorobenzeneboronic acid (38 mg, 0.27 mmol, 3.0 eq), Tetrakis(triphenylphosphine) palladium(0) (21 mg, 0.02 mmol, 0.22 eq) in 1.5 ml t-BuOH and 0.4 ml 1M K₂CO₃ solution, was irradiated in a microwave oven (Emrys™ Optimizer, Biotage) at 110° C. for 15 min. TLC analysis showed a clean reaction and MS analysis showed MH⁺=419/463/519. The reaction mixture was purified on silica gel using 10% MeOH in DCM as eluent to give (S)-tert-Butyl 3-((9-(3-(2-fluorophenyl)benzyl)-8-oxo-8,9-dihydro-7H-purin-2-ylamino)methyl)pyrrolidine-1-carboxylate (44 mg, 95%; Intermediate 27) as a yellow oil. ¹HNMR (CDCl₃, 300 MHz): δ 9.90 (brs, 1H), 7.80 (s, 1H), 7.65 (s, 1H), 7.40 (m, 4H), 7.30 (m, 1H), 7.10 (m, 2H), 5.30 (brs, 1H), 5.05 (s, 2H), 3.55-3.30 (m, 3H), 3.25 (m, 1H), 3.10 (m, 1H), 2.45 (m, 1H), 2.00-1.80 (m, 2H), 1.60 (m, 1H), 1.45 (s, 9H).

### Step 2 (R)-9-(3-(2-Fluorophenyl)benzyl)-2-(pyrrolidin-3-ylmethylamino)-7H-purin-8(9H)-one TFA salt (28)

(S)-tert-Butyl 3-((9-(3-(2-fluorophenyl)benzyl)-8-oxo-8,9-dihydro-7H-purin-2-ylamino)methyl)pyrrolidine-1-carboxylate [44 mg, 0.085 mmol; Intermediate 27] was dissolved in 2 ml of a DCM/TFA (2:1) mixture and stirred for 3 h. MS analysis was consistent with complete consumption of starting material (removal of the N-Boc group) and the formation of product. The mixture was concentrated in vacuo to give (R)-9-(3-(2-Fluorophenyl)benzyl)-2-(pyrrolidin-3-ylmethylamino)-7H-purin-8(9H)-one [45 mg, quantitative; 28] as a pale yellow oil in the TFA salt form. ¹HNMR (CD₃OD, 300 MHz): δ 7.75 (s, 1H), 7.60 (s, 1H), 7.50-7.30 (m, 5H), 7.25-7.15 (m, 2H), 5.15 (s, 2H), 3.60 (dd, 1H), 3.55 (dd, 1H), 3.40-3.30 (m, 2H), 3.20 (m, 1H), 3.00 (m, 1H), 2.70 (m, 1H), 2.15 (m, 1H), 1.75 (m, 1H). LCMS: 4.6 min, M⁺=419.3.

### Preparation of (R)-9-(3-(3-fluorophenyl)benzyl)-2-(pyrrolidin-3-ylmethylamino)-7H-purin-8(9H)-one TFA salt (30)

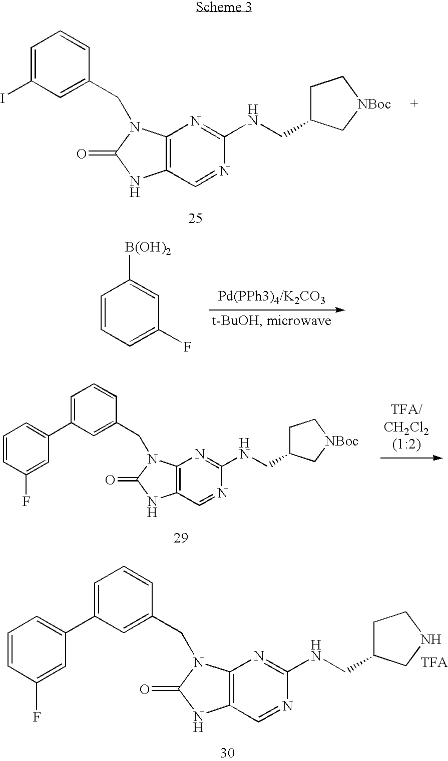

Step 1 (S)-tert-Butyl 3-((9-(3-(3-fluorophenyl)benzyl)-8-oxo-8,9-dihydro-7H-purin-2-ylamino)methyl)pyrrolidine-1-carboxylate (29)

A mixture of (S)-tert-Butyl 3-((9-(3-iodobenzyl)-8-oxo-8,9-dihydro-7H-purin-2-ylamino)methyl)pyrrolidine-1-carboxylate (35 mg, 0.064 mmol, 1.0 eq; Intermediate 25) and 3-Fluorobenzeneboronic acid (37 mg, 0.26 mmol, 4.1 eq), Tetrakis(triphenylphosphine) palladium(0) (20 mg, 0.019 mmol, 0.3 eq) in 1.5 ml t-BuOH and 0.4 ml 1M K₂CO₃ solution, was irradiated in a microwave oven (Emrys™ Optimizer, Biotage) 110° C. for 15 min. TLC analysis showed a clean reaction and MS analysis showed MH⁺=419/463/519. The reaction mixture was purified on silica gel using 10% MeOH in DCM as eluent to give (S)-tert-Butyl 3-((9-(3-(3-fluorophenyl)benzyl)-8-oxo-8,9-dihydro-7H-purin-2-ylamino)methyl)pyrrolidine-1-carboxylate (25 mg, 76%; Intermediate 29) as a pale yellow oil. ¹HNMR (CDCl₃, 300 MHz): δ 7.80 (s, 1H), 7.65 (s, 1H), 7.45 (brs, 2H), 7.40-7.25 (m, 3H), 7.20 (d, 1H), 7.00 (t, 1H), 5.40 (brs, 1H), 5.05 (s, 2H), 3.55-3.30 (m, 4H), 3.30 (m, 1H), 3.10 (m, 1H), 2.50 (m, 1H), 1.95 (m, 1H), 1.60 (m, 1H), 1.45 (s, 9H).

### Step 2 (R)-9-(3-(3-Fluorophenyl)benzyl)-2-(pyrrolidin-3-ylmethylamino)-7H-purin-8(9H)-one TFA salt (30)

(S)-tert-Butyl 3-((9-(3-(3-fluorophenyl)benzyl)-8-oxo-8,9-dihydro-7H-purin-2-ylamino)methyl)pyrrolidine-1-carboxylate [25 mg, 0.048 mmol; Intermediate 29] was dissolved in 1.5 ml of a mixture of DCM/TFA (2:1) and stirred for 1.5 h at room temperature. MS analysis was consistent with complete consumption of starting material (removal of the N-Boc group) and the formation of product. The mixture was concentrated in vacuo to give (R)-9-(3-(3-Fluorophenyl)benzyl)-2-(pyrrolidin-3-ylmethylamino)-7H-purin-8(9H)-one [26 mg, quantitative; 30] as a pale yellow foam in the TFA salt form. ¹HNMR (CD₃OD, 300 MHz): δ 7.80 (s, 1H), 7.75 (s, 1H), 7.60 (s, 1H), 7.45 (m, 4H), 7.35 (d, 1H), 7.10 (m, 1H), 5.15 (s, 2H), 3.60 (dd, 1H), 3.55 (dd, 1H), 3.45-3.30 (m, 2H), 3.20 (m, 1H), 3.05 (m, 1H), 2.70 (m, 1H), 2.15 (m, 1H), 1.75 (m, 1H).

### Preparation of (R)-9-(3-iodo-2-methylbenzyl)-2-(pyrrolidin-3-ylmethylamino)-7H-purin-8(9H)-one TFA salt (35)

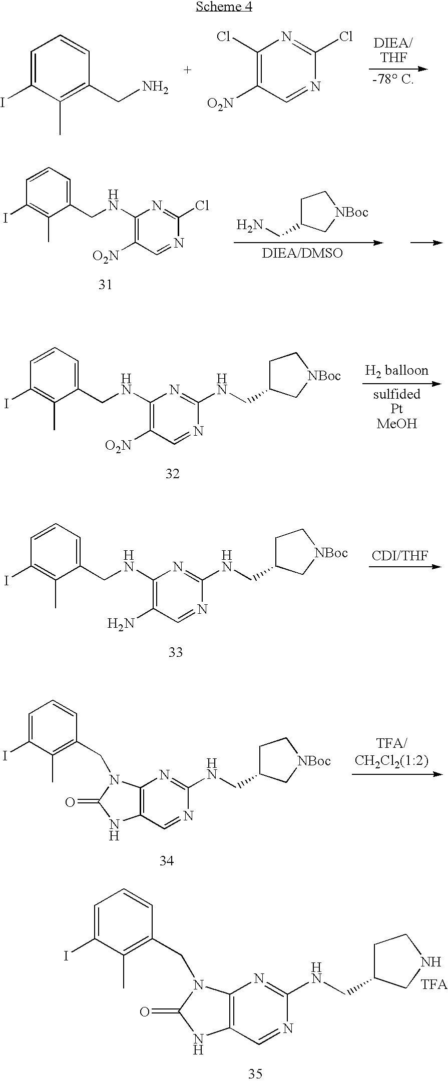

### Step 1 N-(3-iodo-2-methylbenzyl)-2-chloro-5-nitropyrimidin-4-amine (31)

To a solution of 2,4-Dichloro-5-nitropyrimidine (0.78 g, 4.0 mmol, 1.0 eq) in THF (25 mL) at −78° C. was added Diisopropylethylamine (0.78 g, 6.0 mmol, 1.5 eq), followed by 3-Iodo-2-methylbenzylamine (1.0 g, 4.0 mmol, 1.0 eq). The mixture was stirred for 2 h at −78° C. and warmed up to room temperature over 3 h. The mixture was then diluted with brine and extracted with EtOAc; the organic layer was washed with brine and concentrated in vacuo. The resulting slush was purified on silica using 20% EtOAc in hexanes as eluent to give N-(3-Iodo-2-methylbenzyl)-2-chloro-5-nitropyrimidin-4-amine (0.70 g; Intermediate 31) as a pale yellow solid. ¹HNMR (CDCl₃, 300 MHz): δ 9.05 (s, 1H), 8.50 (brs, 1H), 7.85 (dd, 1H), 7.30 (d, 1H), 6.90 (t, 1H), 4.85 (d, 2H), 2.50 (s, 3H).

### Step 2 (S)-tert-Butyl 3-((4-(3-iodo-2-methylbenzylamino)-5-nitropyrimidin-2-ylamino)methyl)pyrrolidine-1-carboxylate (32)

To a solution of N-(3-Iodo-2-methylbenzyl)-2-chloro-5-nitropyrimidin-4-amine (0.65 g, 1.61 mmol, 1.0 eq; Intermediate 31) in DMSO (9 ml) was added Diisopropylethylamine (0.44 g, 3.4 mmol, 2.1 eq), followed by (S)-tert-Butyl 3-(aminomethyl)pyrrolidine-1-carboxylate (0.38 g, 1.9 mmol, 1.18 eq) in DMSO (2 ml). The mixture was stirred at room temperature for 1 h when TLC analysis showed the consumption of starting material and MS analysis showed MH⁺=469/513/569. The mixture was diluted with brine (20 ml), extracted with EtOAc (3×20 ml), dried on MgSO₄ and concentrated under reduced pressure. The residue was purified on silica gel using 30% EtOAc in hexanes as eluent to give (S)-tert-Butyl 3-((4-(3-iodo-2-methylbenzylamino)-5-nitropyrimidin-2-ylamino)methyl)pyrrolidine-1-carboxylate [0.88 g, 96%; Intermediate 32) as a brown slush. ¹HNMR (CDCl₃, 300 MHz): δ 8.95 (s, 1H), 8.75 (s, 1H), 7.80 (d, 1H), 7.20 (d, 1H), 6.85 (t, 1H), 5.90 (s, 1H), 4.75 (d, 2H), 3.60-3.20 (m, 6H), 3.00 (m, 1H), 2.50 (s, 3H), 2.40 (m, 1H), 1.90 (m, 1H), 1.45 (s, 9H).

### Step 3 (S)-tert-Butyl 3-((4-(3-iodo-2-methylbenzylamino)-5-aminopyrimidin-2-ylamino)methyl)pyrrolidine-1-carboxylate (33)

To a solution of (S)-tert-Butyl 3-((4-(3-iodo-2-methylbenzylamino)-5-nitropyrimidin-2-ylamino)methyl)pyrrolidine-1-carboxylate (0.38 g, 0.67 mmol, 1.0 eq; Intermediate 32) in MeOH (6 ml) was added 5% sulfided Pt/C (100 mg). The mixture was stirred under a hydrogen atmosphere (balloon; 1 ATM) overnight when TLC analysis indicated the consumption of starting material and MS analysis showed MH⁺=539.4. The reaction mixture was filtered through a pad of celite and the filtrate was concentrated in vacuo to afford (S)-tert-Butyl 3-((4-(3-iodo-2-methylbenzylamino)-5-aminopyrimidin-2-ylamino)methyl)pyrrolidine-1-carboxylate [0.30 g, 83%; Intermediate 33] as a crude brown solid that was used directly in the subsequent cyclization step without purification.

Step 4 (S)-tert-Butyl 3-((9-(3-iodo-2-methylbenzyl)-8-oxo-8,9-dihydro-7H-purin-2-ylamino)methyl)pyrrolidine-1-carboxylate (34)

Crude (S)-tert-Butyl 3-((4-(3-iodo-2-methylbenzylamino)-5-aminopyrimidin-2-ylamino)methyl)pyrrolidine-1-carboxylate (0.30 g, 0.56 mmol; Intermediate 33) was dissolved in THF (4 ml) and 1,1′-carbonyldiimidazole (0.22 g, 1.36 mmol, 2.42 eq) was added. The mixture was then stirred at room temperature overnight. The reaction was concentrated to dryness in vacuo. The resulting slush was taken up in DCM and water, shaken, separated, dried on MgSO₄, filtered and concentrated. The crude residue was purified by silica gel chromatography using 10% MeOH in DCM as eluent to give (S)-tert-Butyl 3-((9-(3-iodo-2-methylbenzyl)-8-oxo-8,9-dihydro-7H-purin-2-ylamino)methyl)pyrrolidine-1-carboxylate [210 mg, 67%; Intermediate 34] as a brown solid. ¹HNMR (CDCl₃, 300 MHz): δ 9.65 (brs, 1H), 7.85 (s, 1H), 7.75 (d, 1H), 7.20 (d, 1H), 6.80 (t, 1H), 5.20 (brs, 1H), 5.05 (s, 2H), 3.55-3.20 (m, 4H), 3.10 (m, 1H), 2.60 (s, 3H), 2.45 (m, 1H), 1.90 (m, 2H), 1.60 (m, 1H), 1.45 (s, 9H).

### Step 5 (R)-9-(3-Iodo-2-methylbenzyl)-2-(pyrrolidin-3-ylmethylamino)-7H-purin-8(9H)-one TFA salt (35)

(S)-tert-Butyl 3-((9-(3-iodo-2-methylbenzyl)-8-oxo-8,9-dihydro-7H-purin-2-ylamino)methyl)pyrrolidine-1-carboxylate [20 mg, 0.035 mmol; Intermediate 34] was dissolved in 1.5 ml of a mixture of DCM/TFA (2:1) for 40 min at room temperature. MS analysis was consistent with the consumption of starting material and the appearance of product (M⁺=465.3). The reaction was concentrated in vacuo to give (R)-9-(3-Iodo-2-methylbenzyl)-2-(pyrrolidin-3-ylmethylamino)-7H-purin-8(9H)-one TFA salt [19 mg, 35] as a pale yellow film in the TFA salt form. ¹HNMR (CD₃OD, 300 MHz): δ 7.85 (d, 1H), 7.80 (s, 1H), 7.30 (d, 1H), 6.95 (t, 1H), 5.15 (s, 2H), 3.60 (m, 1H), 3.60-3.30 (m, 4H), 3.25 (m, 1H), 3.10 (m, 1H), 2.80 (m, 1H), 2.65 (s, 3H), 2.15 (m, 1H), 1.80 (m, 1H). LCMS: 4.15 min, MH⁺=465.2.

### Preparation of (R)-9-(3-(3-fluorophenyl)-2-methylbenzyl)-2-(pyrrolidin-3-ylmethylamino)-7H-purin-8(9H)-one TFA salt (37)

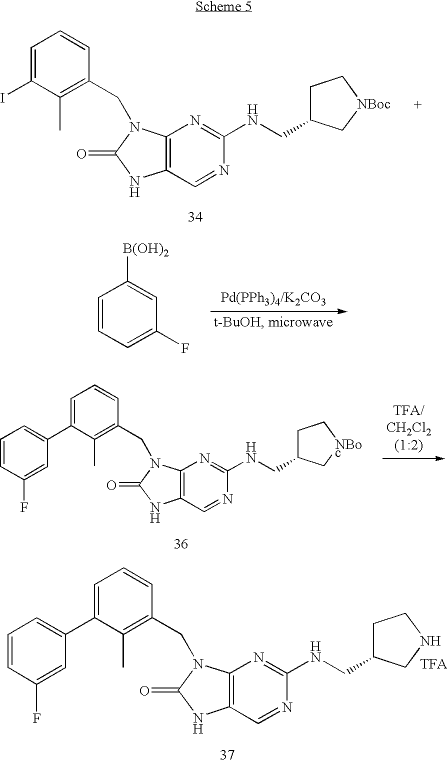

Step 1 (S)-tert-Butyl 3-((9-(3-(3-fluorophenyl)-2-methylbenzyl)-8-oxo-8,9-dihydro-7H-purin-2-ylamino)methyl)pyrrolidine-1-carboxylate (36)

A mixture of (S)-tert-Butyl 3-((9-(3-iodo-2-methylbenzyl)-8-oxo-8,9-dihydro-7H-purin-2-ylamino)methyl)pyrrolidine-1-carboxylate (28 mg, 0.05 mmol, 1.0 eq; Intermediate 34) and 3-Fluorobenzeneboronic acid (35 mg, 0.25 mmol, 5.0 eq), Tetrakis(triphenylphosphine) palladium(0) (17 mg, 0.016 mmol, 0.3 eq) in 1.2 ml t-BuOH and 0.5 ml 1M K₂CO₃ solution, was irradiated in a microwave oven (Emrys™ Optimizer, Biotage) at 110° C. for 15 min. MS analysis showed MH⁺=433/477/533. The reaction mixture was purified on silica gel using 10% MeOH in DCM as eluent to give (S)-tert-Butyl 3-((9-(3-(3-fluorophenyl)-2-methylbenzyl)-8-oxo-8,9-dihydro-7H-purin-2-ylamino)methyl)pyrrolidine-1-carboxylate [20 mg, 76%; Intermediate 36] as a pale yellow oil. ¹HNMR (CDCl₃, 300 MHz): δ 9.60 (brs, 1H), 7.85 (s, 1H), 7.35 (m, 1H), 7.15 (m, 3H), 7.00 (m, 3H), 5.20 (brs, 1H), 5.10 (s, 2H), 3.55-3.20 (m, 5H), 3.10 (m, 1H), 2.45 (m, 1H), 2.35 (s, 3H), 1.95 (m, 1H), 1.60 (m, 1H), 1.45 (s, 9H).

### Step 2 (R)-9-(3-(3-Fluorophenyl)-2-methylbenzyl)-2-(pyrrolidin-3-ylmethylamino)-7H-purin-8(9H)-one TFA salt (37)

(S)-tert-Butyl 3-((9-(3-(3-fluorophenyl)-2-methylbenzyl)-8-oxo-8,9-dihydro-7H-purin-2-ylamino)methyl)pyrrolidine-1-carboxylate [20 mg, 0.12 mmol; Intermediate 36] was dissolved in 1.5 ml of a mixture of DCM/TFA (2:1) and stirred for 40 min. MS analysis showed MH⁺=433.4. The reaction was concentrated in vacuo to give (R)-9-(3-(3-Fluorophenyl)-2-methylbenzyl)-2-(pyrrolidin-3-ylmethylamino)-7H-purin-8(9H)-one [24 mg; 37] as pale yellow foam in the TFA salt form. ¹HNMR (CD₃OD, 300 MHz): δ 7.80 (s, 1H), 7.45 (m, 1H), 7.20 (m, 3H), 7.10 (t, 1H), 7.00 (d, 1H), 5.20 (s, 2H), 3.60-3.30 (m, 4H), 3.25 (m, 1H), 3.00 (m, 1H), 2.70 (m, 1H), 2.35 (s, 3H), 2.15 (m, 1H), 1.75 (m, 1H).

### Preparation of 9-((R)-1-(2,5-Dichlorophenyl)ethyl)-2-((R)-pyrrolidin-3-ylmethylamino)-7H-purin-8(9H)-one TFA salt (45)

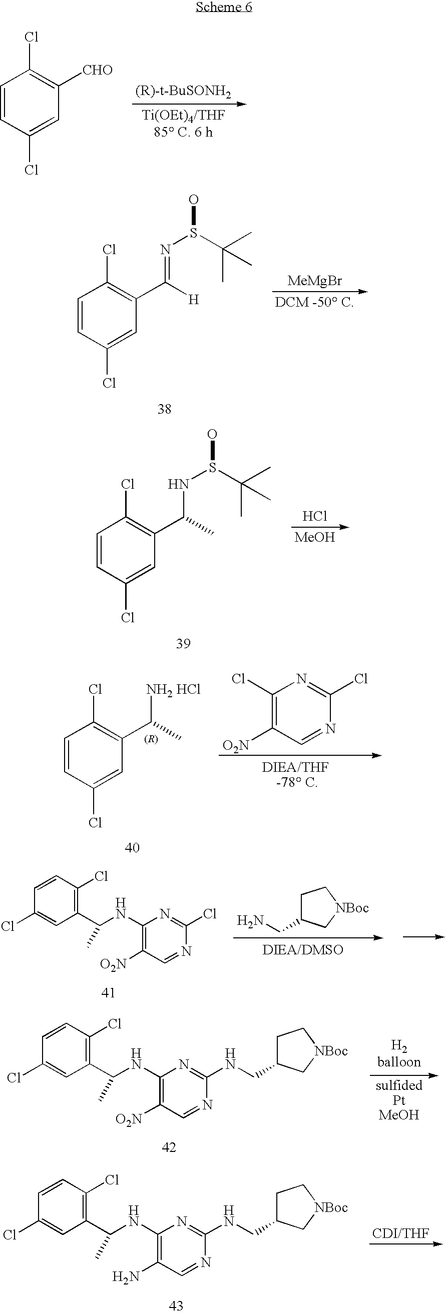

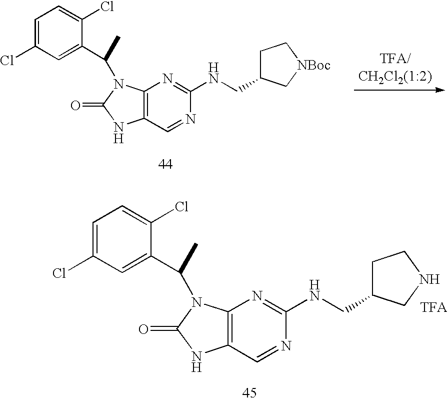

### Step 1 (R)-2-methyl-N-(2,5-dichlorophenyl)propane-2-sulfinamide (38)

To a solution of (R)-(+)-2-methyl-2-propane sulfinamide (1.73 g, 14 mmol, 1 eq) and 2,5-dichlorobenzaldehyde (2.5 g, 14 mmol, 1 eq) in anhydrous THF (8 mL) at room temperature was added Ti(OEt)₄ (3.8 g, 16.8 mmol, 1.2 eq). The mixture was heated at 80° C. for 6 h to give a complete reaction by TLC analysis. After cooling to room temperature, the mixture was diluted with brine under vigorous stirring, and the resulting suspension was filtered through a pad of Celite and the solid was washed several times with EtOAc. The filtrate was washed with water, brine, dried (Na₂SO₄), and concentrated in vacuo. (R)-2-methyl-N-(2,5-dichlorophenyl)propane-2-sulfinamide [3.2 g; Intermediate 38] was obtained as a pale yellow solid. ¹HNMR (CDCl₃, 300 MHz): δ 8.95 (s, 1H), 8.0 (s, 1H), 7.40 (s, 2H), 1.30 (s, 9H).

Step 2 N—((R)-1-(2,5-dichlorophenyl)ethyl)-2-methylpropane-2-sulfinamide (39)

To a solution of (R)-2-methyl-N-(2,5-dichlorophenyl)propane-2-sulfinamide (3.2 g, 11.5 mmol, 1 eq; Intermediate 38) in dichloromethane (50 ml) at −65° C. was injected MeMgBr (3 M in ether, 6 ml, 18 mmol, 1.56 eq) dropwise via syringe over 5 min. The reaction was stirred for 20 min and warmed up to room temperature over 30 min. TLC analysis showed complete consumption of starting material. The reaction was quenched with saturated NH₄Cl, filtered through a pad of Celite and washed with dichloromethane. The filtrate was separated and the organic layer was washed with brine, dried over Na₂SO₄, filtered and concentrated in vacuo to give 3.3 g of crude yellow residue. Chromatography on silica gel using 30% EtOAc in hexanes as eluent gave N—((R)-1-(2,5-dichlorophenyl)ethyl)-2-methylpropane-2-sulfinamide [2.4 g, 71%; Intermediate 39] as a colorless oil. ¹HNMR (CDCl₃, 300 MHz): δ 7.40 (d, 1H), 7.30 (d, 1H), 7.20 (dd, 1H), 5.0 (m, 1H), 3.35 (brs, 1H), 1.55 (d, 3H), 1.25 (s, 9H). LCMS: t_{R}=6.7 min, single peak, MH⁺=294.1/296.1/298.1 (2 Cl isotopes).

### Step 3 (R)-1-(2,5-dichlorophenyl)ethanamine hydrochloride (40)

To a stirred solution of N—((R)-1-(2,5-dichlorophenyl)ethyl)-2-methylpropane-2-sulfinamide (1.4 g, 4.8 mmol, 1 eq; Intermediate 39) in MeOH (3 ml) was added dropwise 4N HCl in dioxane (3 ml, 12 mmol, 2.5 eq). The reaction was stirred for 6 h and MS analysis showed MH⁺=173.3 and 190.2 (both with 2Cl isotopes). The reaction mixture was concentrated in vacuo, and triturated with MeOH, ether, and hexanes to give (R)-1-(2,5-dichlorophenyl)ethanamine [1.2 g, 100%; Intermediate 40] as an off-white fluffy powder in the hydrochloride salt form. ¹HNMR (CD₃OD, 300 MHz): δ 7.63 (d, J=2.4 Hz, 1H), 7.55 (d, J=8.7 Hz, 1H), 7.45 (dd, J=2.4 Hz, 8.7 Hz, 1H), 4.9 (m, 1H), 1.62 (d, J=6.9 Hz, 3H). HPLC: t_{R}=5.06 min, single peak.

### Step 4 (R)-2-Chloro-N-(1-(2,5-dichlorophenyl)ethyl)-5-nitropyrimidin-4-amine (41)

To a solution of 2,4-Dichloro-5-nitropyrimidine (0.25 g, 1.31 mmol, 1.14 eq) in THF (2 mL) at −78° C. was added a mixture of Diisopropylethylamine (0.36 g, 2.79 mmol, 2.4 eq) and (R)-1-(2,5-dichlorophenyl)ethanamine hydrochloride (0.25 g, 1.15 mmol, 1.0 eq; Intermediate 40) in THF (4 ml). The reaction was stirred for 1.5 h and warmed up to room temperature over 1 h to give complete consumption of starting material by TLC analysis. The mixture was diluted with DCM and water, shaken, and separated; the organic layer was washed with brine, dried on MgSO₄, filtered and concentrated in vacuo. The resulting crude residue was purified on silica gel using 20% EtOAc in hexanes as eluent to give (R)-2-chloro-N-(1-(2,5-dichlorophenyl)ethyl)-5-nitropyrimidin-4-amine [0.34 g, 85%; Intermediate 41] as a yellow oil. ¹HNMR (CDCl₃, 300 MHz): δ 9.05 (s, 1H), 8.75 (brd, 1H), 7.35 (d, 1H), 7.30 (d, 1H), 7.20 (dd, 1H), 5.75 (p, 1H), 1.65 (d, 3H). MH⁺=347.2/349.3/351.4 (3Cl).

### Step 5 (S)-tert-Butyl 3-((4-((R)-1-(2,5-dichlorophenyl)ethylamino)-5-nitropyrimidin-2-ylamino)methyl)pyrrolidine-1-carboxylate (42)

To a solution of (R)-2-Chloro-N-(1-(2,5-dichlorophenyl)ethyl)-5-nitropyrimidin-4-amine (0.17 g, 0.49 mmol, 1.0 eq; Intermediate 41) in DMSO (1 ml) was added a mixture of Diisopropylethylamine (0.13 g, 1.0 mmol, 2.0 eq) and (S)-tert-butyl 3-(aminomethyl)pyrrolidine-1-carboxylate (127 mg, 0.63 mmol, 1.3 eq) in DMSO (1 ml). The reaction was stirred at room temperature overnight. TLC analysis showed a major new entity, and MS analysis confirmed the presence of product (M⁺=411/455/511). The mixture was diluted with brine (10 ml), extracted with EtOAc (3×10 ml), dried on MgSO₄, filtered and concentrated under reduced pressure. The residue was purified on silica gel using 20% EtOAc in hexanes as eluent to give (S)-tert-Butyl 3-((4-((R)-1-(2,5-dichlorophenyl)ethylamino)-5-nitropyrimidin-2-ylamino)methyl)pyrrolidine-1-carboxylate [0.22 g, 88%; Intermediate 42] as a yellow solid. ¹HNMR (CDCl₃, 300 MHz): δ 8.85 (s, 1H), 8.75 (d, 1H), 7.20 (m, 2H), 7.10 (m, 1H), 6.65 (brd, 1H), 5.50 (p, 1H), 3.40-3.05 (m, 5H), 2.95 (m, 1H), 2.15 (m, 1H), 1.90 (m, 1H), 1.45 (s, 9H).

### Step 6 (S)-tert-Butyl 3-((5-amino-4-((R)-1-(2,5-dichlorophenyl)ethylamino)pyrimidin-2-ylamino)methyl)pyrrolidine-1-carboxylate (43)

To a solution of (S)-tert-Butyl 3-((4-((R)-1-(2,5-dichlorophenyl)ethylamino)-5-nitropyrimidin-2-ylamino)methyl)pyrrolidine-1-carboxylate (0.22 g, 0.43 mmol; Intermediate 42) in MeOH (6 ml) was added 5% sulfided Pt/C (100 mg). The mixture was stirred under a hydrogen atmosphere (balloon; 1 ATM) overnight when TLC analysis showed consumption of starting material and MS analysis was consistent with product formation (MH⁺=381/425/481). The reaction mixture was filtered through celite and the filtrate was concentrated in vacuo to afford (S)-tert-Butyl 3-((5-amino-4-((R)-1-(2,5-dichlorophenyl)ethylamino)pyrimidin-2-ylamino)methyl)pyrrolidine-1-carboxylate [0.18 g, 87%; Intermediate 43] as a crude amber residue that was used directly in the subsequent cyclization step.

### Step 7 (S)-tert-Butyl 3-((9-((R)-1-(2,5-dichlorophenyl)ethyl)-8-oxo-8,9-dihydro-7H-purin-2-ylamino)methyl)pyrrolidine-1-carboxylate (44)

Crude (S)-tert-Butyl 3-((5-amino-4-((R)-1-(2,5-dichlorophenyl)ethylamino)pyrimidin-2-ylamino)methyl)pyrrolidine-1-carboxylate (0.18 g, 0.375 mmol; Intermediate 43) was dissolved in THF (3 ml) and 1,1′-carbonyldiimidazole (0.25 g, 1.54 mmol, 4.1 eq) was added. The mixture was stirred at room temperature for 5 h and was then concentrated to dryness in vacuo. The resulting residue was partitioned between DCM and water. The organic phase was separated, dried on MgSO₄, filtered and concentrated by rotoevaporation. The product was isolated by silica gel chromatography using 10% MeOH in DCM as eluent to give (S)-tert-Butyl 3-((9-((R)-1-(2,5-dichlorophenyl)ethyl)-8-oxo-8,9-dihydro-7H-purin-2-ylamino)methyl)pyrrolidine-1-carboxylate [58 mg, 31%; Intermediate 44] as a yellow solid. ¹HNMR (CDCl₃, 300 MHz): δ 7.80 (s, 2H), 7.25 (d, 1H), 7.20 (d, 1H), 5.95 (q, 1H), 5.30 (brs, 1H), 3.60-3.10 (m, 5H), 3.05 (m, 1H), 2.45 (m, 1H), 1.95 (m, 1H), 1.90 (d, 3H), 1.65 (m, 1H), 1.45 (s, 9H).

### Step 8 9-((R)-1-(2,5-Dichlorophenyl)ethyl)-2-((R)-pyrrolidin-3-ylmethylamino)-7H-purin-8(9H)-one (45)

(S)-tert-Butyl 3-((9-((R)-1-(2,5-dichlorophenyl)ethyl)-8-oxo-8,9-dihydro-7H-purin-2-ylamino)methyl)pyrrolidine-1-carboxylate (58 mg, 0.114 mmol; Intermediate 44) was dissolved in 4.5 ml of a mixture of DCM/TFA (2:1) for 3 h at room temperature. MS analysis was consistent with the generation of product (M⁺=407/409). The mixture was concentrated in vacuo to give 9-((R)-1-(2,5-Dichlorophenyl)ethyl)-2-((R)-pyrrolidin-3-ylmethylamino)-7H-purin-8(9H)-one [92 mg; 45] as an amber foam in the TFA salt form. ¹HNMR (CD₃OD, 300 MHz): δ 7.90 (d, 1H), 7.75 (s, 1H), 7.45 (d, 1H), 7.40 (d, 1H), 5.95 (q, 1H), 3.60-3.30 (m, 5H), 3.05 (m, 1H), 2.70 (m, 1H), 2.25 (m, 1H), 1.95 (d, 3H), 1.80 (m, 1H). LCMS: 4.0 min, M⁺=407.2/409.2.

### Preparation of 9-(2,5-dichlorobenzyl)-2-(((1 r,4r)-4-(aminomethyl)cyclohexyl)methylamino)-7H-purin-8(9H)-one TFA salt (50)

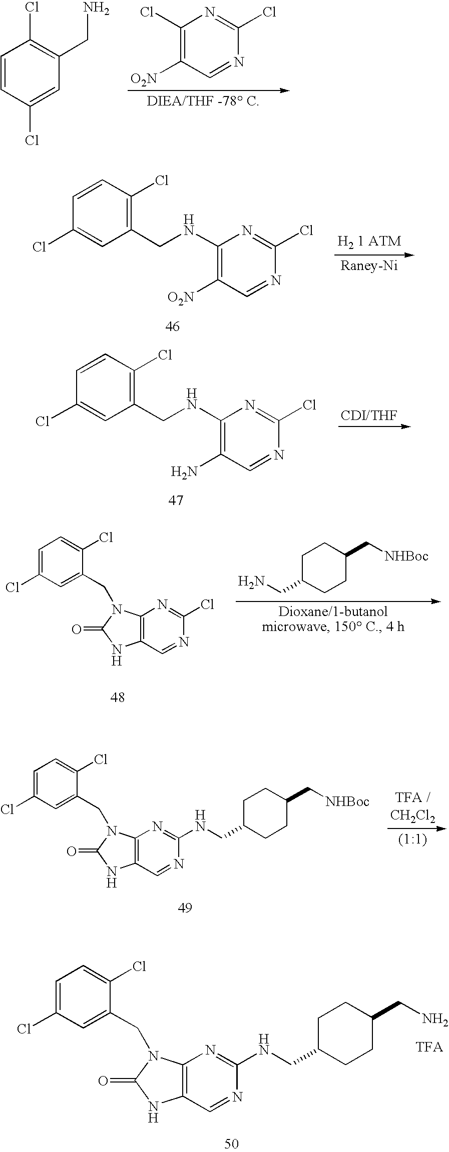

### Step 1. N-(2,5-dichlorobenzyl)-2-chloro-5-nitropyrimidin-4-amine (46)

To a solution of 2,4-Dichloro-5-nitropyrimidine (1.1 g, 5.7 mmol) in THF (15 mL) at −78° C. was added dropwise a mixture of Diisopropylethylamine (2.2 mL, 12.5 mmol) and 2,5-Dichlorobenzylamine (1.0 g, 5.7 mmol) in THF (10 ml). The resulting suspension was stirred for 50 minutes at −78° C. and was then allowed to warm up to room temperature over 30 minutes. The mixture was poured into water, extracted with EtOAc, dried over Na₂SO₄, filtered and concentrated by rotoevaporation to a crude brown residue. Silica gel chromatography using a gradient of 10-15% EtOAc in petroleum ether as eluent gave N-(2,5-Dichlorobenzyl)-2-chloro-5-nitropyrimidin-4-amine [1 g; Intermediate 46] as an off-white solid. LC-MS: Rₜ 6.35 min, m/e 332.6/334.6; ¹HNMR (CDCl₃, 300 MHz): δ 9.07 (s, 1H), 8.82 (br s, 1H), 7.46 (d, 1H), 7.31 (m, 1H), 7.27 (m, 1H), 4.89 (d, 2H).

### Step 2. N4-(2,5-dichlorobenzyl)-2-chloropyrimidine-4,5-diamine (47)

A mixture of N-(2,5-Dichlorobenzyl)-2-chloro-5-nitropyrimidin-4-amine [5.5 g, 16.5 mmol; Intermediate 46], THF (150 mL), and Raney-Ni (aqueous slurry) was placed under a hydrogen atmosphere (balloon; 1 ATM) at room temperature for 2.5 h. The mixture was filtered over a pad of Celite and the filtrate was concentrated under reduced pressure to give crude N4-(2,5-dichlorobenzyl)-2-chloropyrimidine-4,5-diamine [4.8 g; Intermediate 47] which was used in the subsequent cyclization step without purification.

### Step 3. 9-(2,5-dichlorobenzyl)-2-chloro-7H-purin-8(9H)-one (48)

A mixture of crude N4-(2,5-dichlorobenzyl)-2-chloropyrimidine-4,5-diamine [0.36 g, 1.17 mmol; Intermediate 47], THF (15 mL), and 1,1′-carbonyldiimidazole (0.57 g, 3.51 mmol) was stirred overnight at room temperature. The mixture was diluted with water [20 mL] and extracted with EtOAc. The combined organic phases were dried on Na₂SO₄, filtered and concentrated in vacuo to a crude yellow solid. This was triturated with EtOAc [10 mL] and filtered to give 9-(2,5-dichlorobenzyl)-2-chloro-7H-purin-8(9H)-one [1 g; Intermediate 48]. LC-MS: Rₜ 5.42 min, m/e 328.6/330.6/332.6; ¹HNMR (DMSO-d₆, 300 MHz): δ 8.18 (s, 1H), 7.53 (d, 1H), 7.41 (dd, 1H), 7.31 (d, 1H), 5.01 (s, 2H).

### Step 4. tert-Butyl ((1r,4r)-4-((9-(2,5-dichlorobenzyl)-8-oxo-8,9-dihydro-7H-purin-2-ylamino)methyl)cyclohexyl)methylcarbamate (49)

A microwave tube was charged with 9-(2,5-dichlorobenzyl)-2-chloro-7H-purin-8(9H)-one (66 mg, 0.200 mmol; Intermediate 48), tert-Butyl ((1r,4r)-4-(aminomethyl)cyclohexyl)methylcarbamate (106 mg, 0.44 mmol), 1,4-Dioxane (0.5 ml) and 1-Butanol (0.5 ml). The mixture was sealed and irradiated in a microwave oven (Emrys™ Optimizer, Biotage) to 150° C. for 4 hours. The mixture was diluted with Ethyl acetate (15 mL) and washed with saturated aqueous NH₄Cl (1×3 mL), brine (2×3 mL), dried over MgSO₄, filtered and concentrated by rotoevaporation. Silica gel chromatography (hexane/ethyl acetate; 1:1-1:2) afforded tert-Butyl ((1r,4r)-4-((9-(2,5-dichlorobenzyl)-8-oxo-8,9-dihydro-7H-purin-2-ylamino)methyl)cyclohexyl)methylcarbamate (36 mg, 0.067 mmol, 34%; Intermediate 49). MS: m/e 535.4, 537.4, 539.4 (M=1); ¹H NMR (CDCl₃) δ 8.23 (b, 1H), 7.87 (s, 1H), 7.32 (d, 1H), 7.18 (dd, 1H), 7.14 (d, 1H), 5.10 (s, 2H), 4.98 (b, 1H), 4.56 (b, 1H), 3.20 (m, 2H), 2.96 (m, 2H), 1.77 (m, 4H), 1.2-1.7 (m, 2H), 1.44 (s, 9H), 0.91 (m, 4H).

### Step 5. 9-(2,5-dichlorobenzyl)-2-(((1r,4r)-4-(aminomethyl)cyclohexyl)methylamino)-7H-purin-8(9H)-one (50)

To a solution of tert-Butyl ((1r,4r)-4-((9-(2,5-dichlorobenzyl)-8-oxo-8,9-dihydro-7H-purin-2-ylamino)methyl)cyclohexyl)methylcarbamate (36 mg, 0.067 mmol; Intermediate 49) in DCM (1 mL) was added TFA (1 mL). The mixture was then stirred at room temperature for 1 h. The mixture was concentrated under reduced pressure. The resultant residue was dissolved in methanol (1 ml) and purified by prep RP-HPLC to give 9-(2,5-dichlorobenzyl)-2-(((1r,4r)-4-(aminomethyl)cyclohexyl)methylamino)-7H-purin-8(9H)-one [30 mg, 0.055 mmol, 80%; 50] as an off-white solid in the TFA salt form. MS: m/e 435.1, 437.1 (M+1); ¹H NMR (CD₃OD) δ 7.74 (s, 1H), 7.45 (d, 1H), 7.41 (b, 1H), 7.40 (dd, 1H), 5.15 (s, 2H), 3.26 (d, 2H), 2.78 (d, 2H), 1.83 (m, 4H), 1.57 (b, 2H), 1.00 (m, 4H).

### Preparation of 9-(2,5-Dichlorobenzyl)-2-(((1s,4s)-4-aminocyclohexyl)methylamino)-7H-purin-8(9H)-one TFA salt (52)

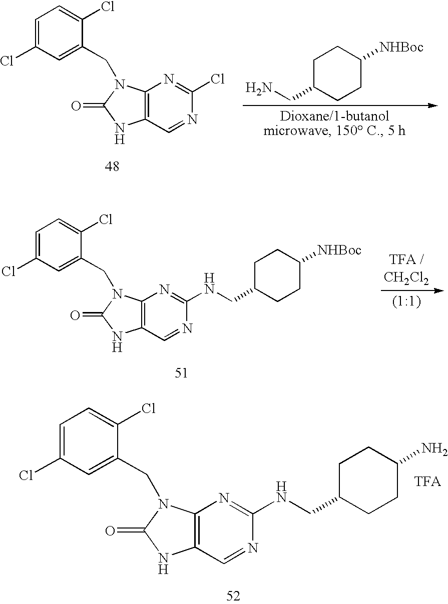

### Step 1. tert-Butyl (1s,4s)-4-((9-(2,5-dichlorobenzyl)-8-oxo-8,9-dihydro-7H-purin-2-ylamino)methyl)cyclohexylcarbamate (51)

A microwave tube was charged with 9-(2,5-dichlorobenzyl)-2-chloro-7H-purin-8(9H)-one (66 mg, 0.200 mmol; Intermediate 48), tert-butyl (1s,4s)-4-(aminomethyl)cyclohexylcarbamate (100 mg, 0.44 mmol), 1,4-Dioxane (0.5 ml) and 1-Butanol (0.5 ml). The mixture was sealed and irradiated in a microwave oven (Emrys™ Optimizer, Biotage) to 150° C. in a microwave for 5 hours. The mixture was diluted with ethyl acetate (15 mL) and washed with saturated aqueous NH₄Cl(1×3 mL), brine(2×3 mL), dried over MgSO₄, filtered and concentrated by rotoevaporation. The crude product was purified by column chromatography (hexane/ethyl acetate; 1:1-1:2) to obtain tert-Butyl (1s,4s)-4-((9-(2,5-dichlorobenzyl)-8-oxo-8,9-dihydro-7H-purin-2-ylamino)methyl)cyclohexylcarbamate (59 mg, 0.113 mmol, 56%; Intermediate 51). MS: m/e 521.4, 523.4 (M+1); ¹H NMR (CDCl₃) δ 9.05 (b, 1H), 7.88 (s, 1H), 7.33 (d, 1H), 7.20 (dd, 1H), 7.14 (b, 1H), 5.12 (s, 2H), 4.61 (b, 1H), 3.71 (b, 1H), 3.27 (m, 2H), 1.15-1.75 (m, 9H), 1.44 (s, 9H).

### Step 2. 9-(2,5-dichlorobenzyl)-2-(((1s,4s)-4-aminocyclohexyl)methylamino)-7H-purin-8(9H)-one (52)

To a solution of tert-Butyl (1s,4s)-4-((9-(2,5-dichlorobenzyl)-8-oxo-8,9-dihydro-7H-purin-2-ylamino)methyl)cyclohexylcarbamate (59 mg, 0.113 mmol; Intermediate 51) in DCM (1 mL) was added TFA (1 mL). The mixture was then stirred at room temperature for 1 h. The mixture was concentrated under reduced pressure. The resultant residue was dissolved in methanol (1 ml) and purified by prep RP-HPLC to give 9-(2,5-dichlorobenzyl)-2-(((1s,4s)-4-aminocyclohexyl)methylamino)-7H-purin-8(9H)-one [40 mg, 0.075 mmol, 66%; 52] as an off-white solid in the TFA salt form. MS: m/e 421.1, 423.1 (M+1); ¹H NMR (CD₃OD) δ 7.77 (s, 1H), 7.46 (d, 1H), 7.43 (b, 1H), 7.35 (dd, 1H), 5.17 (s, 2H), 3.39 (d, 2H), 1.45-1.95 (m, 10H).

### Preparation of 9-(2,5-Dichlorobenzyl)-2-(((1s,4s)-4-aminocyclohexyl)methylamino)-7H-purin-8(9H)-one TFA salt (52)

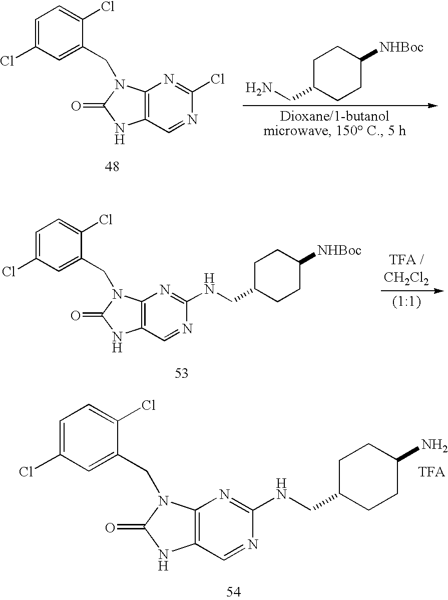

### Step 1. tert-Butyl (1r,4r)-4-((9-(2,5-dichlorobenzyl)-8-oxo-8,9-dihydro-7H-purin-2-ylamino)methyl)cyclohexylcarbamate (53)

A microwave tube was charged with 9-(2,5-dichlorobenzyl)-2-chloro-7H-purin-8(9H)-one (66 mg, 0.200 mmol; Intermediate 48), tert-Butyl (1r,4r)-4-(aminomethyl)cyclohexylcarbamate (100 mg, 0.44 mmol), 1,4-Dioxane (0.5 ml) and 1-Butanol (0.5 ml). The mixture

The mixture was sealed and irradiated in a microwave oven (Emrys™ Optimizer, Biotage) to 150° C. in a microwave for 5 hours. The mixture was diluted with ethyl acetate (15 mL) and washed with saturated aqueous NH₄Cl(1×3 mL), brine(2×3 mL), and dried over MgSO₄. The crude product was purified by column chromatography (hexane/ethyl acetate; 1:1-1:2) to obtain tert-Butyl (1r,4r)-4-((9-(2,5-dichlorobenzyl)-8-oxo-8,9-dihydro-7H-purin-2-ylamino)methyl)cyclohexylcarbamate (12 mg, 0.023 mmol, 20%; Intermediate 53). MS: m/e 521.4, 523.3 (M+1).

### Step 2. 9-(2,5-dichlorobenzyl)-2-(((1r,4r)-4-aminocyclohexyl)methylamino)-7H-purin-8(9H)-one TFA salt (54)

To a solution of tert-Butyl (1r,4r)-4-((9-(2,5-dichlorobenzyl)-8-oxo-8,9-dihydro-7H-purin-2-ylamino)methyl)cyclohexylcarbamate (12 mg, 0.023 mmol; Intermediate 53) in DCM (1 mL) was added TFA (1 mL). The mixture was then stirred at room temperature for 1 h. The mixture was concentrated under reduced pressure. The resultant residue was dissolved in methanol (1 ml) and purified by prep RP-HPLC to give 9-(2,5-dichlorobenzyl)-2-(((1r,4r)-4-aminocyclohexyl)methylamino)-7H-purin-8(9H)-one (8.7 mg, 0.016 mmol, 71%; 54] as an off-white solid in the TFA salt form. MS: m/e 421.0, 423.1 (M+1); ¹H NMR (CD₃OD) δ 7.75 (s, 1H), 7.45 (d, 1H), 7.41 (b, 1H), 7.35 (dd, 1H), 5.15 (s, 2H), 3.27 (d, 2H), 3.02 (m, 1H), 2.03 (m, 2H), 1.88 (m, 2H), 1.56 (m, 1H), 1.35 (m, 2H), 1.08 (m, 2H).

### PKC-Theta IMAP Assay

The activity of the compounds described in the present invention may be determined by the following procedure. This procedure describes a kinase assay that measures the phosphorylation of a fluorescently-labeled peptide by full-length human recombinant active PKCθ via fluorescent polarization using commercially available IMAP reagents.

The PKCθ used was made from full-length, human cDNA (accession number LO1087) with an encoded His-6 sequence at the C-terminus. PKCθ was expressed using the baculovirus expression system. The protein was purified with Ni-NTA affinity chromatography yielding a protein with 91% purity.

The substrate for this assay is a fluorescently-labeled peptide having the sequence LHQRRGSIKQAKVHHVK (FITC)-NH₂. The stock solution of the peptide is 2 mM in water.

The IMAP reagents come from the IMAP Assay Bulk Kit, product #R8063 or #R8125 (Molecular Devices, Sunnyvale, Calif.). The kit materials include a 5×IMAP Binding Buffer and the IMAP Binding Reagent. The Binding Solution is prepared as a 1:400 dilution of IMAP Binding Reagent into the 1×IMAP Binding Buffer.

The substrate/ATP buffer for this assay consists of 20 mM HEPES, pH 7.4 with 5 mM MgCl₂, and 0.01% Tween-20. Additionally, the buffer contains 100 nM substrate, 20 μM ATP, and 2 mM DTT which are added fresh just prior to use. The kinase buffer containing the PKCθ consists of 20 mM HEPES, pH 7.4 with 0.01% Tween-20. This buffer also contains.2 ng/μL PKCθ and 2 mM DTT which are added fresh just prior to use.

The plates used are Corning 3710 (Corning Incorporated, Corning, N.Y.). These are non-treated black polystyrene, 384-well with flat-bottoms. The serial dilutions are performed Nunc V-bottom 96-well plates (Cat#442587, Nunc A/S, Roskilde, Denmark).

The assay procedure starts the preparation of stock solutions of compounds at 10 mM in 100% DMSO. The stock solutions and the control compound are serially diluted 1:3.16 a total of 11 times into DMSO (37 μL of compound into 80 μL of DMSO). After the serial dilution has been completed, a further dilution is performed by taking 4 μL compound and adding to 196 μL substrate/ATP Buffer. Then, 10 μL aliquots of the compounds are transferred to the Costar 3710 plate. The kinase reaction is initiated by the addition of 10 μL PKCθ. This reaction is allowed to incubate for 1 hour at ambient temperature. The reaction is then quenched by the addition of 60 μL of Binding Solution. The plate is incubated for an additional 30 minutes at ambient temperature. The assay is measured using an Acquest™ Ultra-HTS Assay Detection System (Molecular Devices) in fluorescence polarization mode using 485 nm excitation and 530 nm emission.

Tables 1, 2 and 3 illustrate several examples of the compounds of the invention. These compounds were synthesized using one of the suitable procedures described above. The molecular weight of the compounds was confirmed by mass spectroscopy (m/z). The compounds of Tables 1, 2 and 3 were tested using the above-described PKCθ IMAP assay.

TABLE 1

| Example No. | Chemistry | Legend |
|---|---|---|
| 1 | 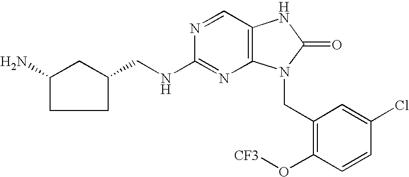 | 1 |
| 2 | 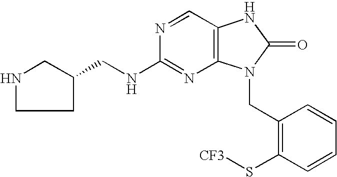 | 1 |
| 3 | 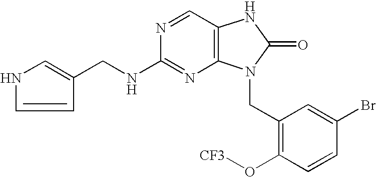 | 1 |
| 4 | 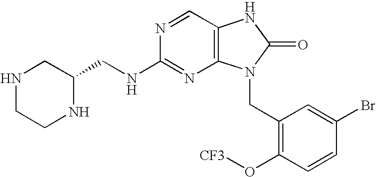 | 1 |
| 5 | 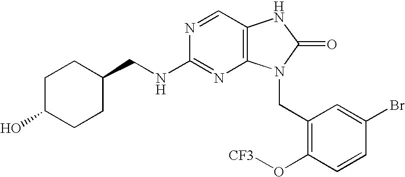 | 1 |
| 6 | 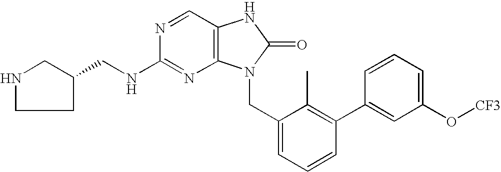 | 1 |
| 7 | 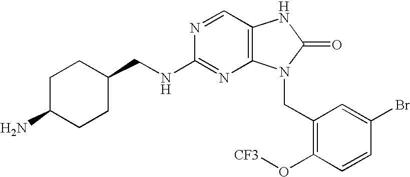 | 1 |
| 8 | 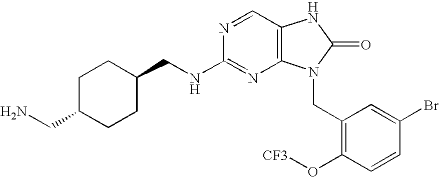 | 1 |
| 9 | 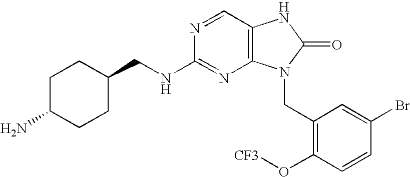 | 1 |
| 10 | 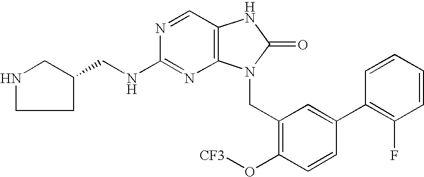 | 1 |
| 11 | 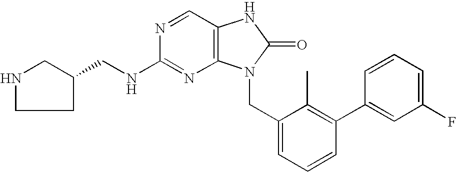 | 1 |
| 12 | 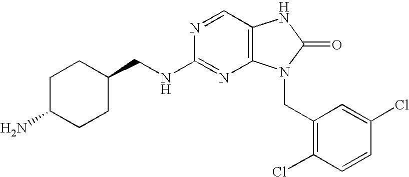 | 1 |
| 13 | 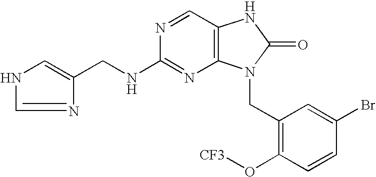 | 1 |
| 14 | 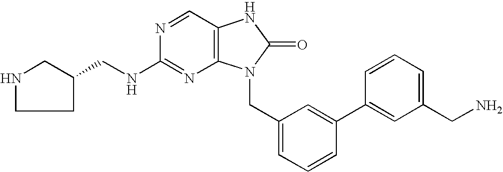 | 1 |
| 15 | 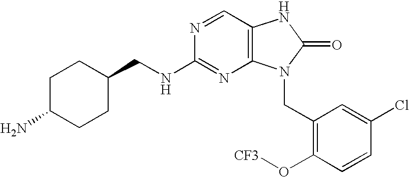 | 1 |
| 16 | 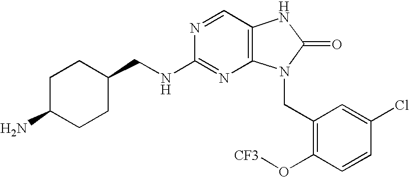 | 1 |
| 17 | 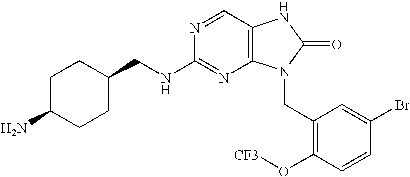 | 1 |
| 18 | 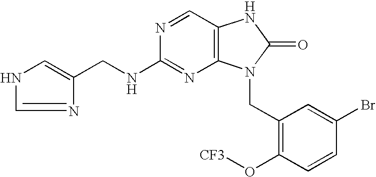 | 1 |
| 19 | 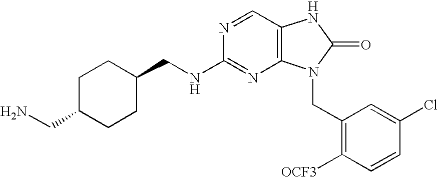 | 1 |
| 20 | 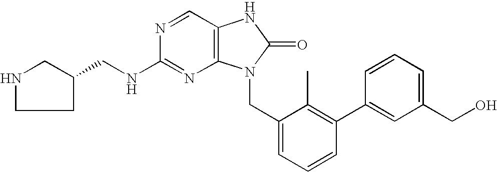 | 1 |
| 21 | 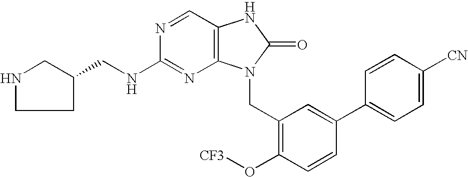 | 1 |
| 22 | 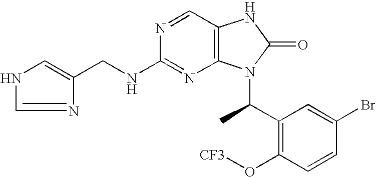 | 1 |
| 23 | 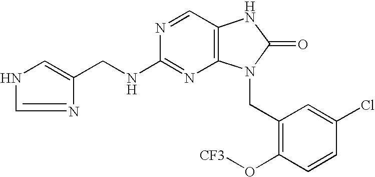 | 1 |
| 24 | 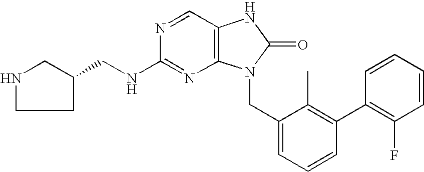 | 1 |
| 25 | 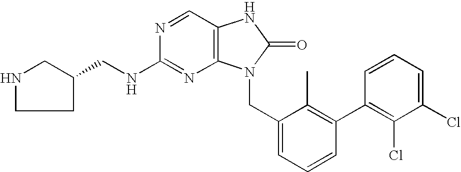 | 1 |
| 26 | 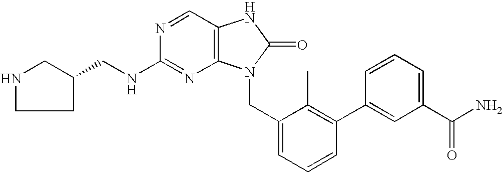 | 1 |
| 27 | 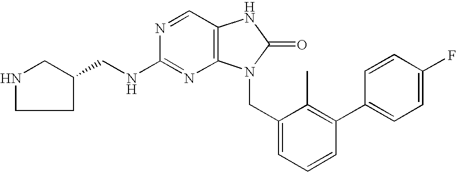 | 1 |
| 28 | 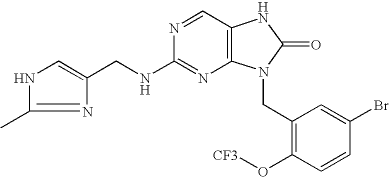 | 1 |
| 29 | 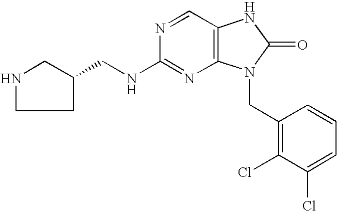 | 1 |
| 30 | 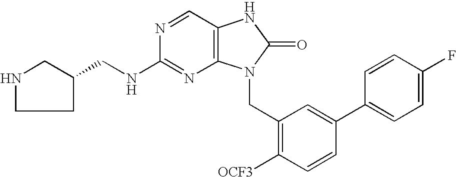 | 1 |
| 31 | 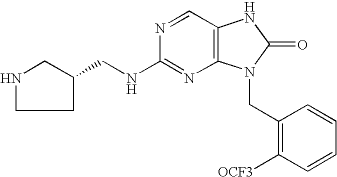 | 1 |
| 32 | 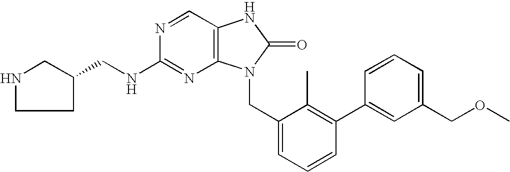 | 1 |
| 33 | 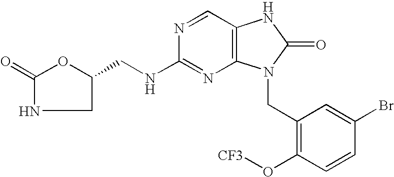 | 1 |
| 34 | 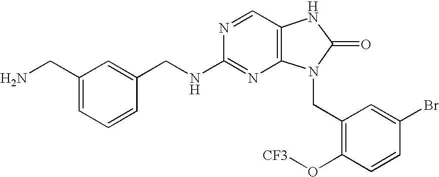 | 1 |
| 35 | 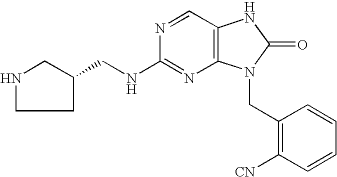 | 1 |
| 36 | 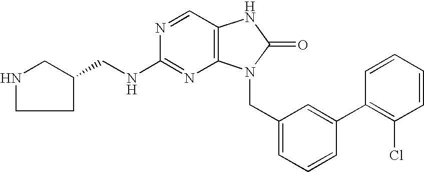 | 1 |
| 37 | 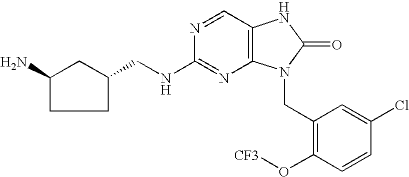 | 1 |
| 38 | 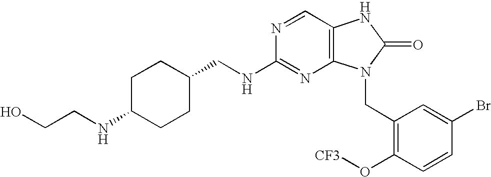 | 1 |
| 39 | 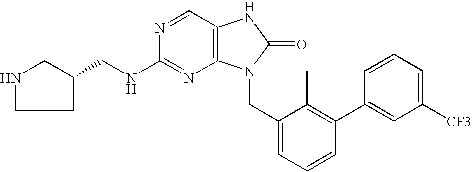 | 1 |
| 40 | 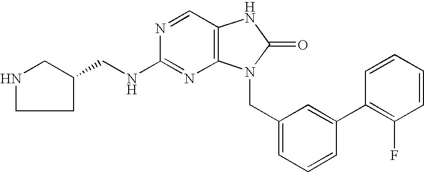 | 1 |
| 41 | 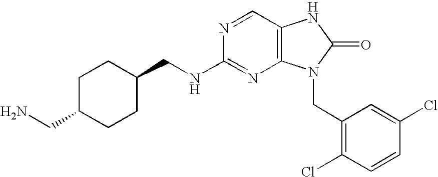 | 1 |
| 42 | 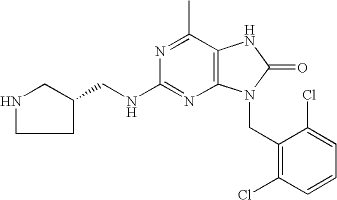 | 1 |
| 43 | 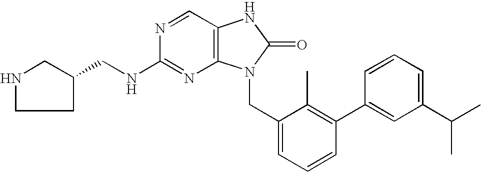 | 1 |
| 44 | 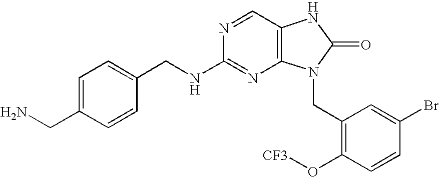 | 1 |
| 45 | 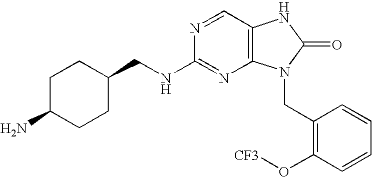 | 1 |
| 46 | 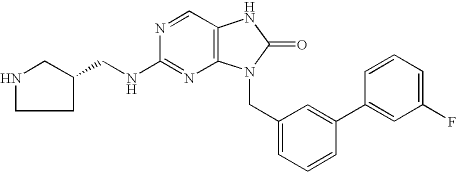 | 1 |
| 47 | 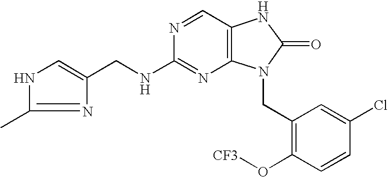 | 1 |
| 48 | 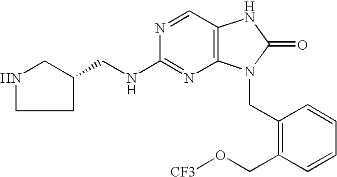 | 1 |
| 49 | 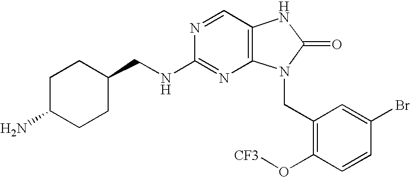 | 1 |
| 50 | 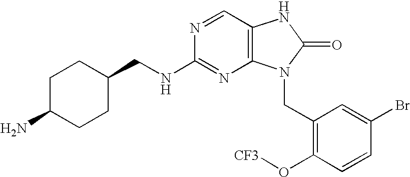 | 1 |
| 51 | 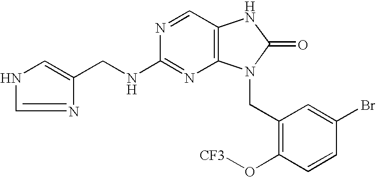 | 1 |
| 52 | 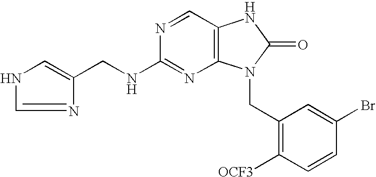 | 1 |
| 53 | 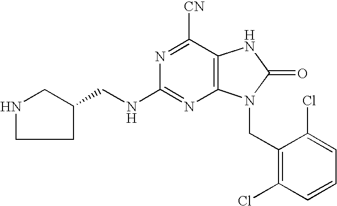 | 1 |
| 54 | 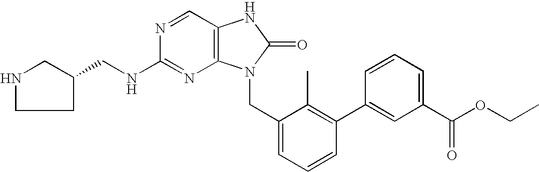 | 1 |
| 55 | 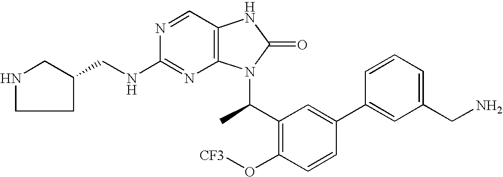 | 1 |
| 56 | 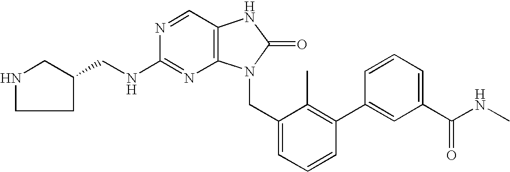 | 1 |
| 57 | 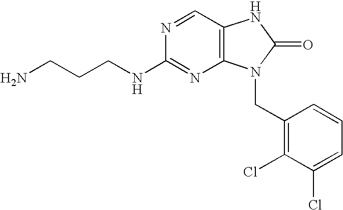 | 1 |
| 58 | 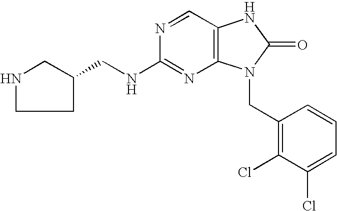 | 1 |
| 59 | 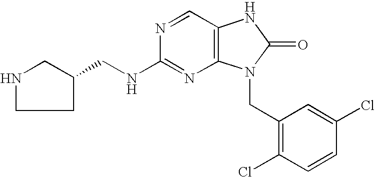 | 1 |
| 60 | 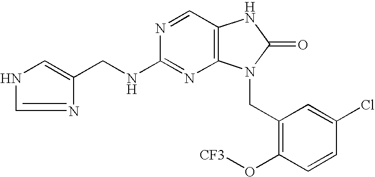 | 1 |
| 61 | 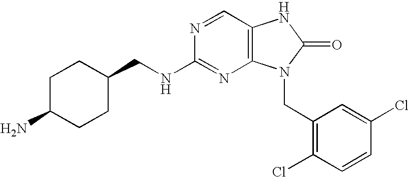 | 1 |
| 62 | 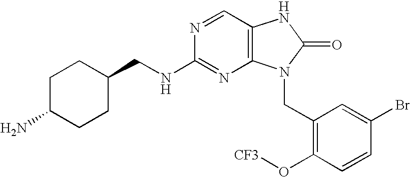 | 1 |
| 63 | 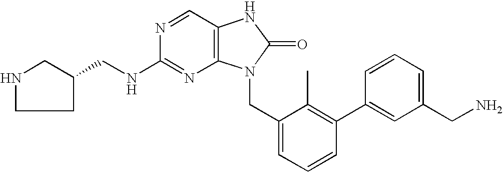 | 1 |
| 64 | 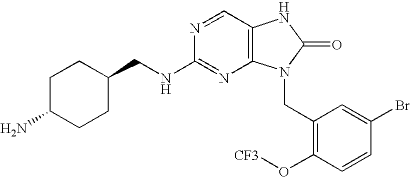 | 1 |
| 65 | 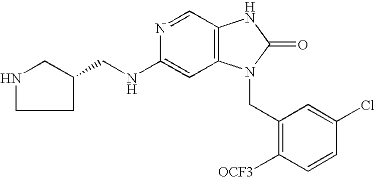 | 1 |
| 66 | 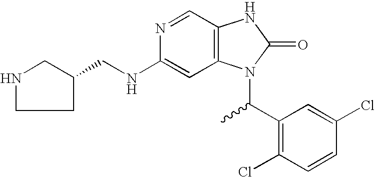 | 1 |
| 67 | 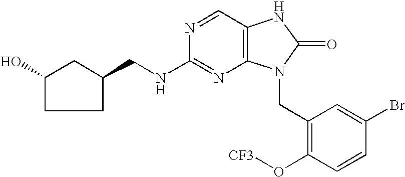 | 2 |
| 68 | 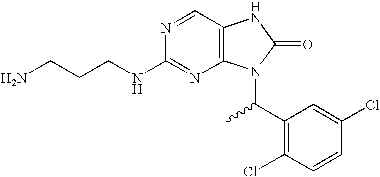 | 2 |
| 69 | 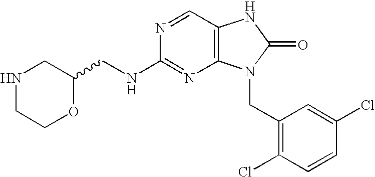 | 2 |
| 70 | 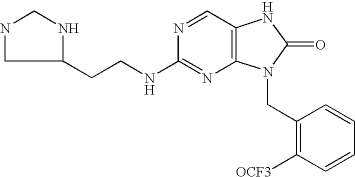 | 2 |
| 71 | 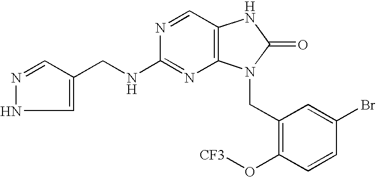 | 2 |
| 72 | 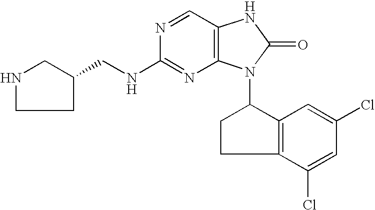 | 2 |
| 73 | 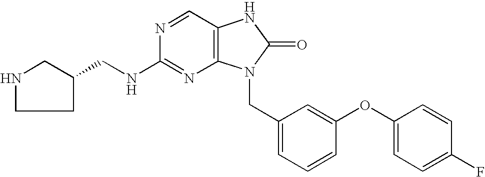 | 2 |
| 74 | 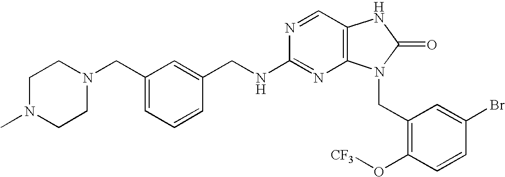 | 2 |
| 75 | 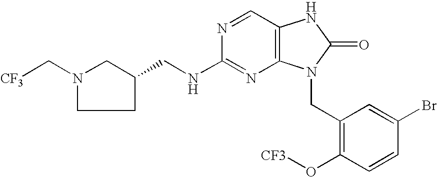 | 2 |
| 76 | 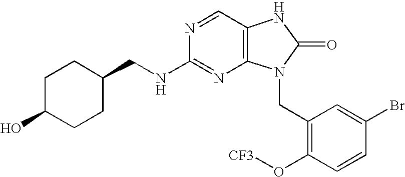 | 2 |
| 77 | 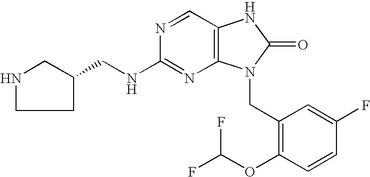 | 2 |
| 78 | 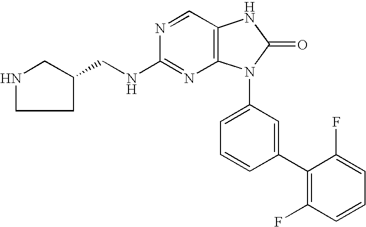 | 2 |
| 79 | 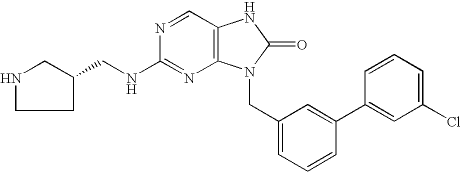 | 2 |
| 80 | 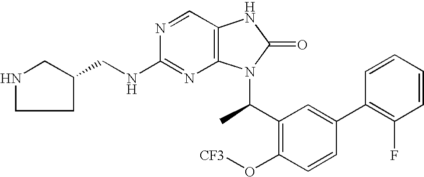 | 2 |
| 81 | 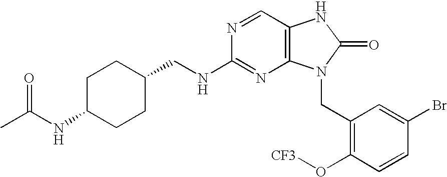 | 2 |
| 82 | 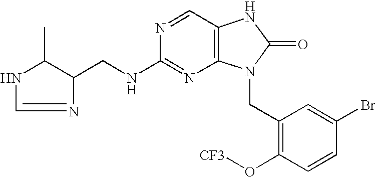 | 2 |
| 83 | 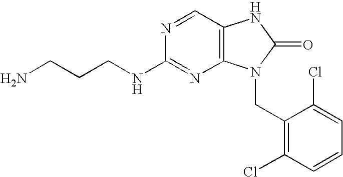 | 2 |
| 84 | 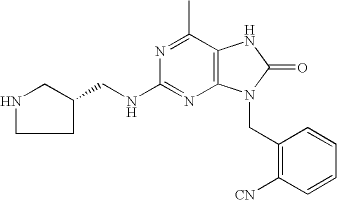 | 2 |
| 85 | 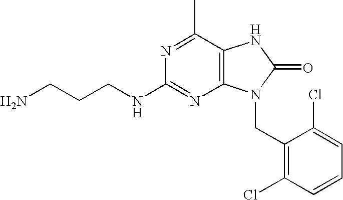 | 2 |
| 86 | 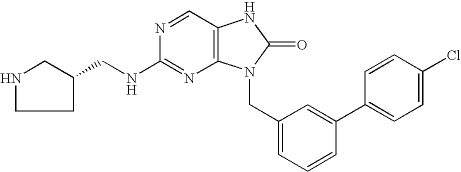 | 2 |
| 87 | 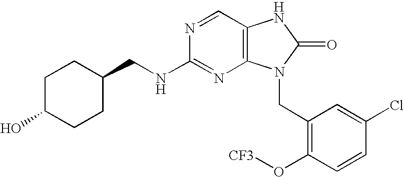 | 2 |
| 88 | 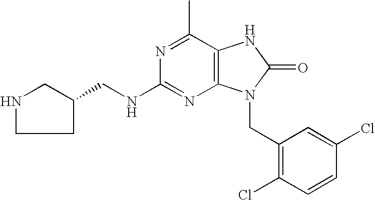 | 2 |
| 89 | 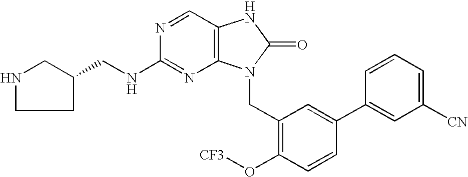 | 2 |
| 90 | 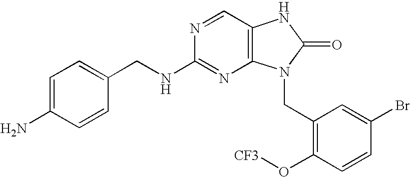 | 2 |
| 91 | 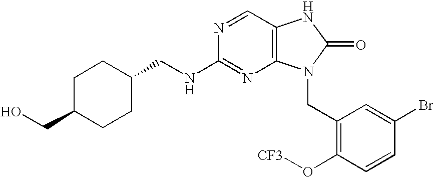 | 2 |
| 92 | 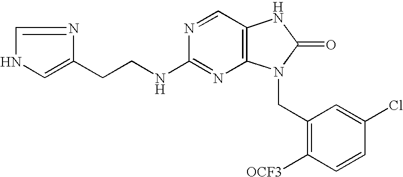 | 2 |
| 93 | 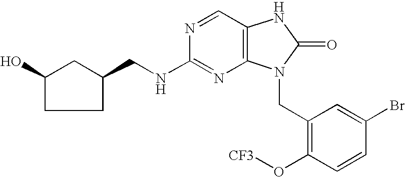 | 2 |
| 94 | 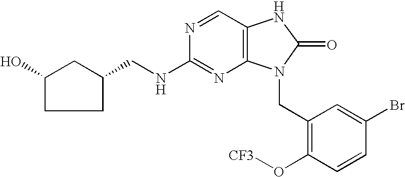 | 2 |
| 95 | 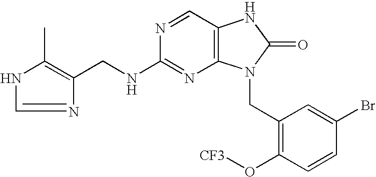 | 2 |
| 96 | 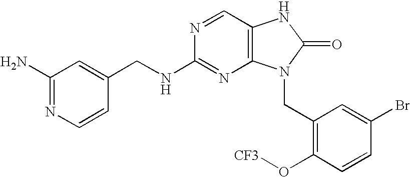 | 2 |
| 97 | 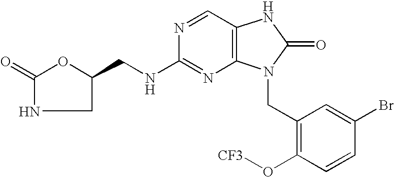 | 2 |
| 98 | 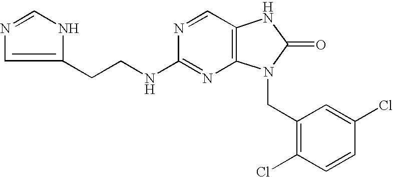 | 2 |
| 99 | 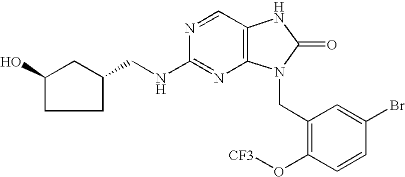 | 2 |
| 100 | 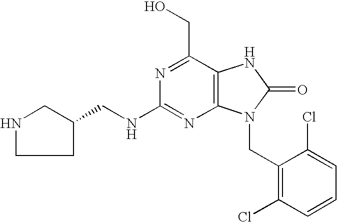 | 2 |
| 101 | 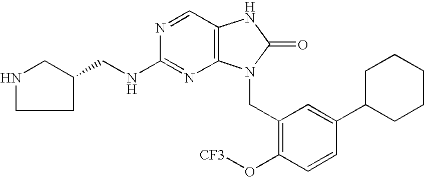 | 3 |
| 102 | 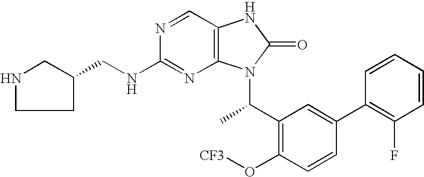 | 3 |
| 103 | 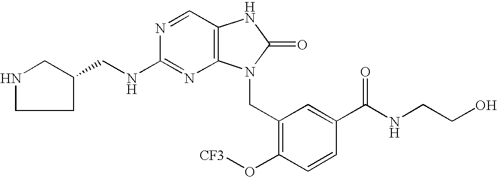 | 3 |
| 104 | 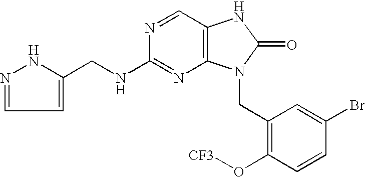 | 3 |
| 105 | 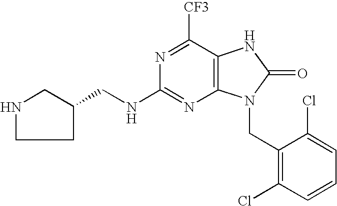 | 3 |
| 106 | 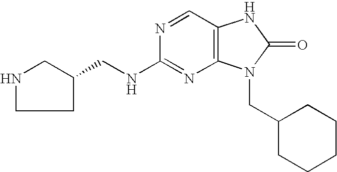 | 3 |
| 107 | 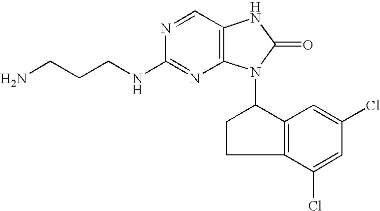 | 3 |
| 108 | 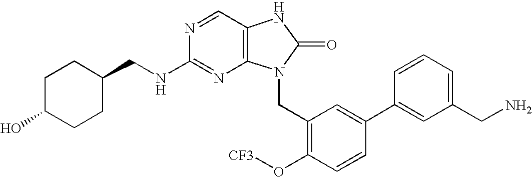 | 3 |
| 109 | 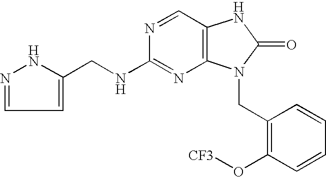 | 3 |
| 110 | 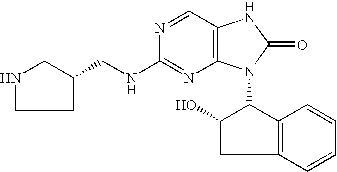 | 3 |
| 111 | 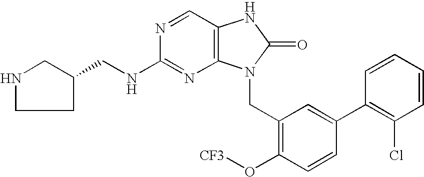 | 3 |
| 112 | 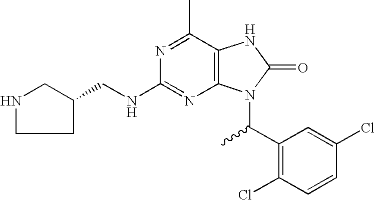 | 3 |
| 113 | 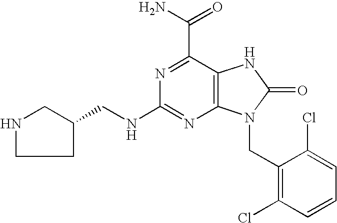 | 3 |
| 114 | 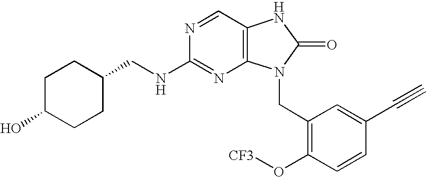 | Not Tested |
| 115 | 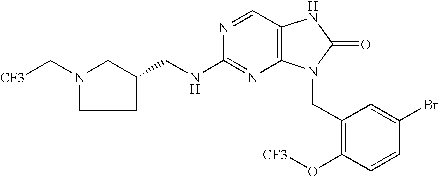 | Not Tested |

TABLE 2

| Example No. | Chemistry | Legend |
|---|---|---|
| 116 | 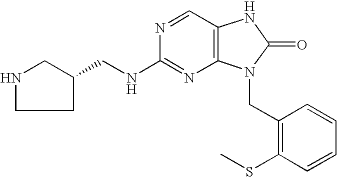 | 1 |
| 117 | 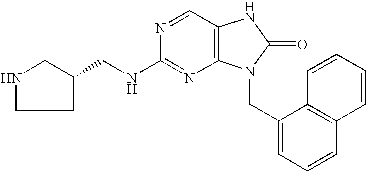 | 1 |
| 118 | 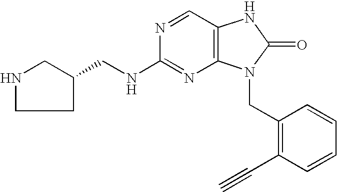 | 1 |
| 119 | 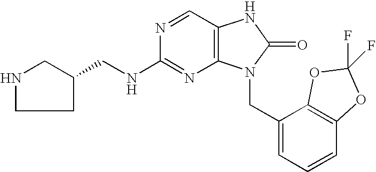 | 1 |
| 120 | 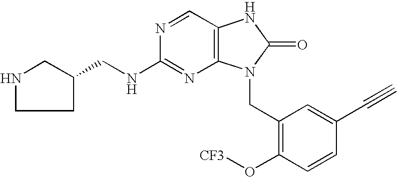 | 1 |
| 121 | 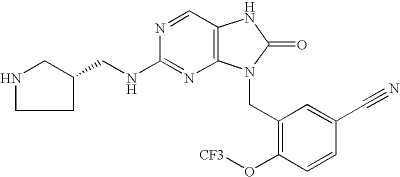 | 1 |
| 122 | 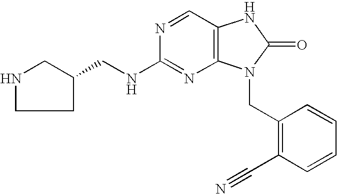 | 1 |
| 123 | 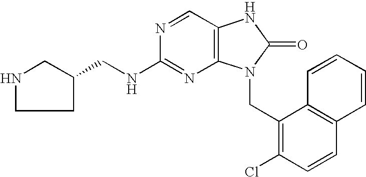 | 1 |
| 124 | 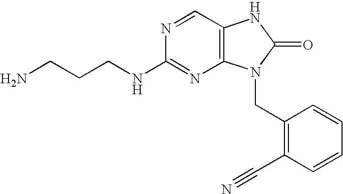 | 1 |
| 125 | 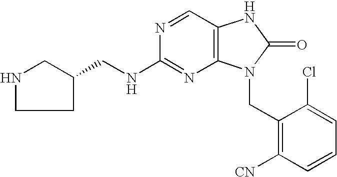 | 1 |
| 126 | 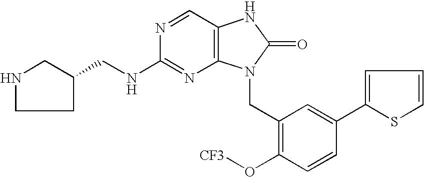 | 2 |
| 127 | 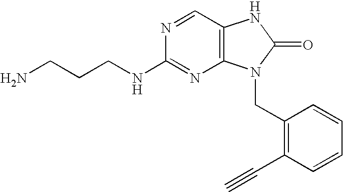 | 2 |
| 128 | 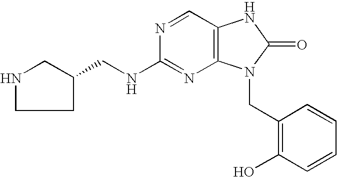 | 2 |
| 129 | 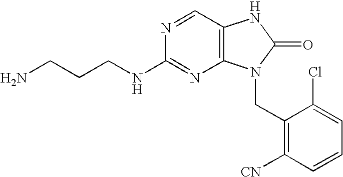 | 2 |
| 130 | 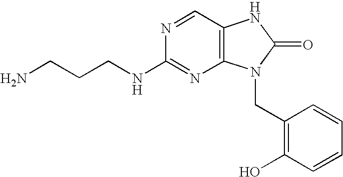 | 2 |
| 131 | 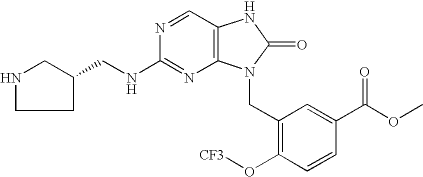 | 2 |
| 132 | 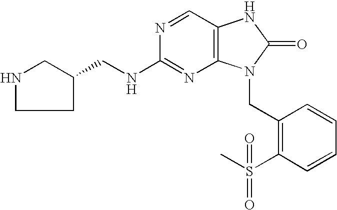 | 2 |
| 133 | 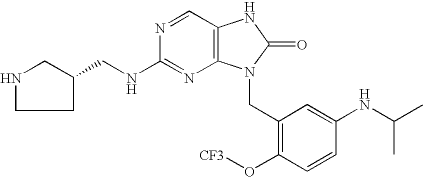 | 2 |
| 134 | 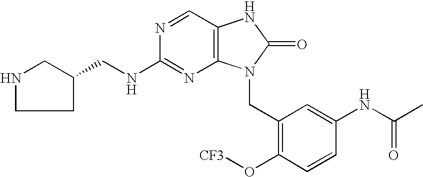 | 2 |
| 135 | 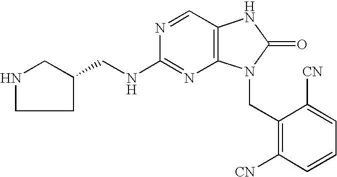 | 2 |
| 136 | 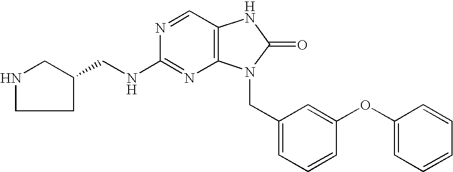 | 3 |

TABLE 3

| Example No. | Chemistry | Legend |
|---|---|---|
| 136A | 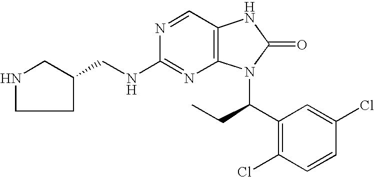 | 1 |
| 137 | 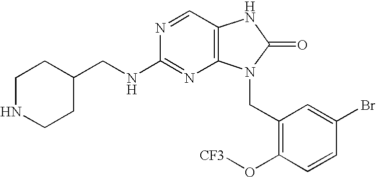 | 1 |
| 138 | 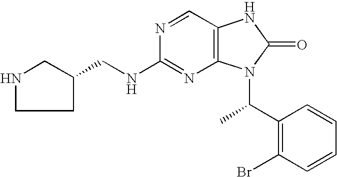 | 1 |
| 139 | 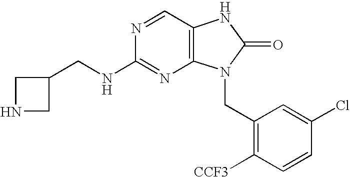 | 1 |
| 140 | 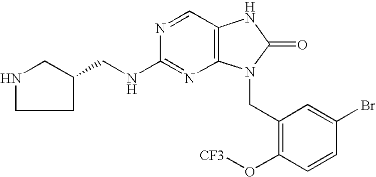 | 1 |
| 141 | 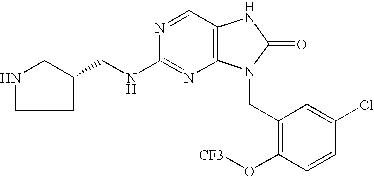 | 1 |
| 142 | 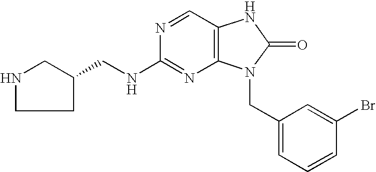 | 1 |
| 143 | 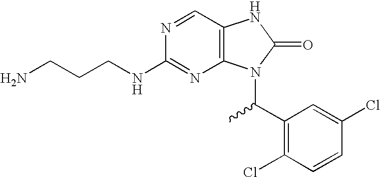 | 1 |
| 144 | 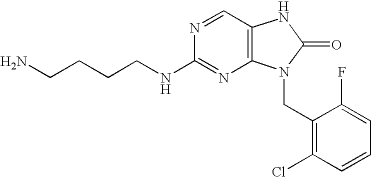 | 1 |
| 145 | 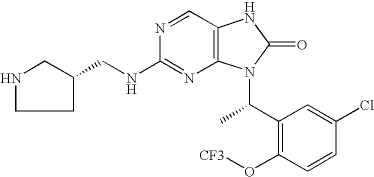 | 1 |
| 146 | 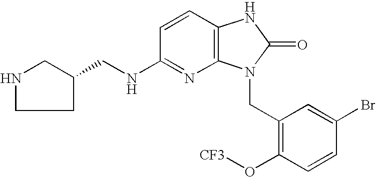 | 1 |
| 147 | 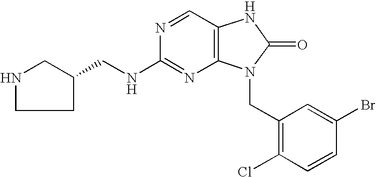 | 1 |
| 148 | 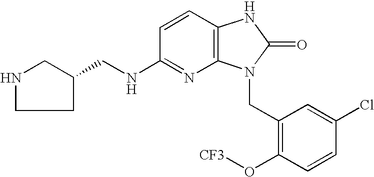 | 1 |
| 149 | 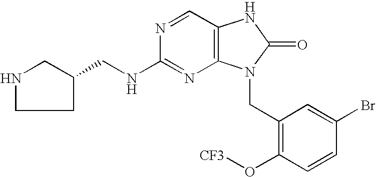 | 1 |
| 150 | 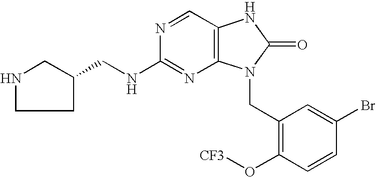 | 1 |
| 151 | 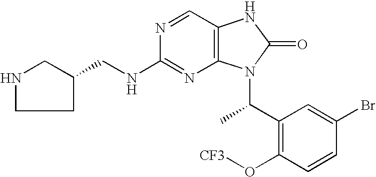 | 1 |
| 152 | 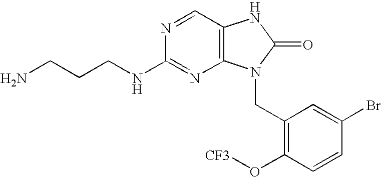 | 1 |
| 153 | 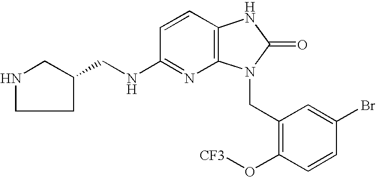 | 1 |
| 154 | 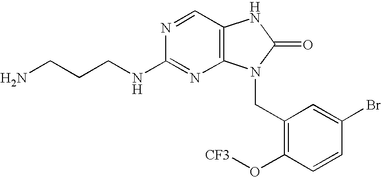 | 1 |
| 155 | 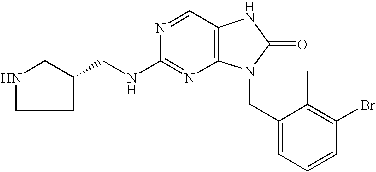 | 1 |
| 156 | 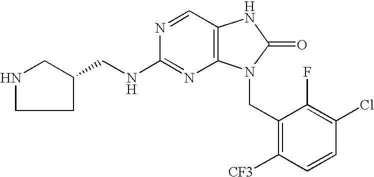 | 1 |
| 157 | 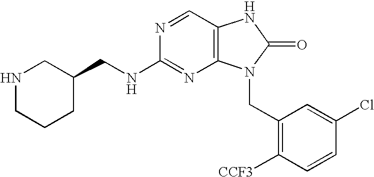 | 1 |
| 158 | 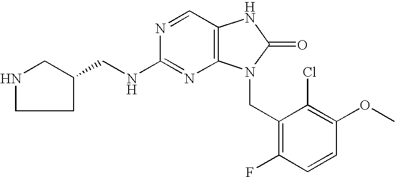 | 1 |
| 159 | 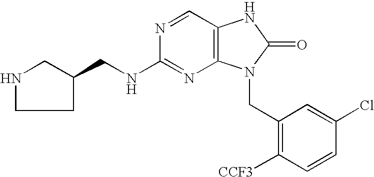 | 1 |
| 160 | 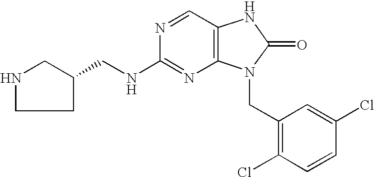 | 1 |
| 161 | 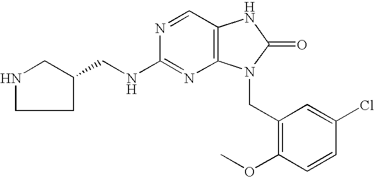 | 1 |
| 162 | 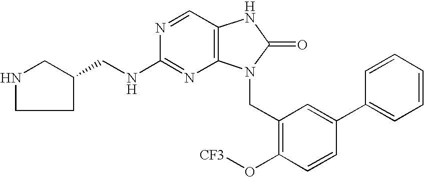 | 1 |
| 163 | 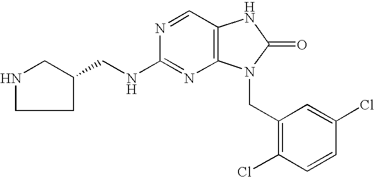 | 1 |
| 164 | 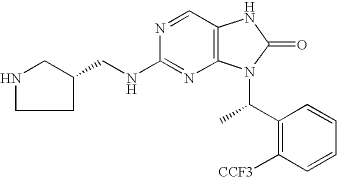 | 1 |
| 165 | 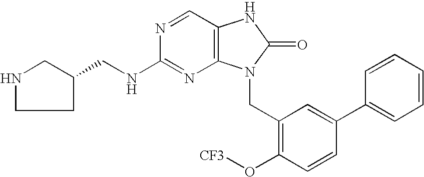 | 1 |
| 166 | 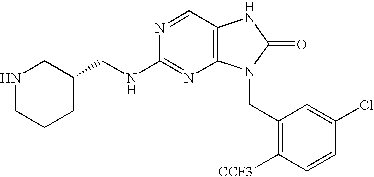 | 1 |
| 167 | 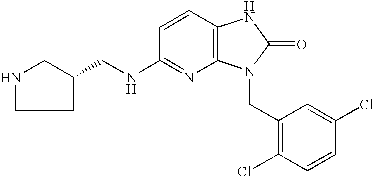 | 1 |
| 168 | 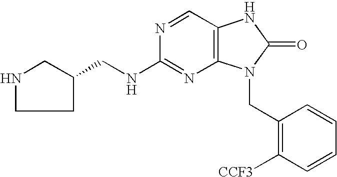 | 1 |
| 169 | 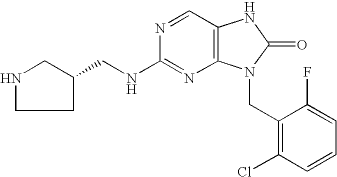 | 1 |
| 170 | 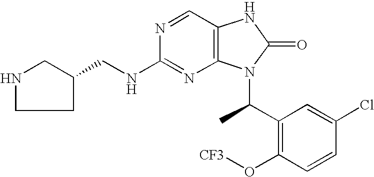 | 1 |
| 171 | 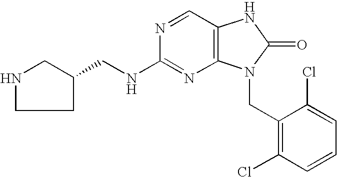 | 1 |
| 172 | 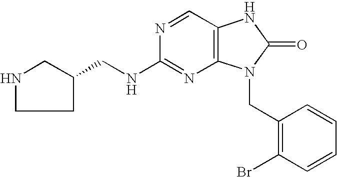 | 1 |
| 173 | 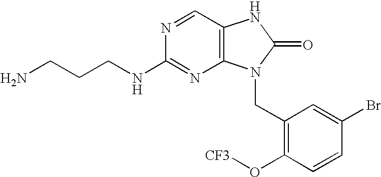 | 1 |
| 174 | 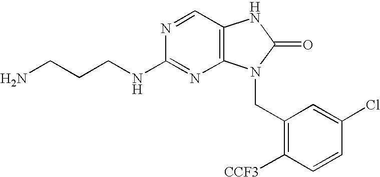 | 1 |
| 175 | 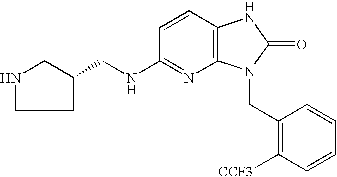 | 1 |
| 176 | 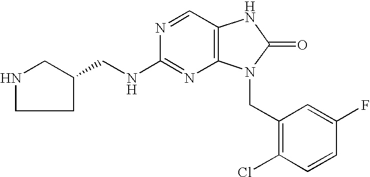 | 1 |
| 177 | 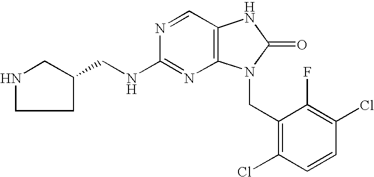 | 1 |
| 178 | 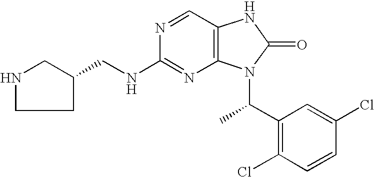 | 1 |
| 179 | 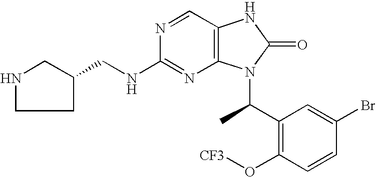 | 1 |
| 180 | 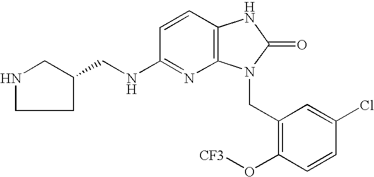 | 1 |
| 181 | 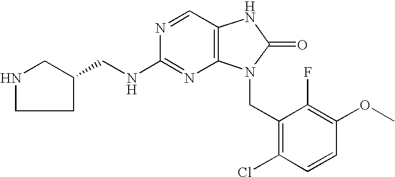 | 1 |
| 182 | 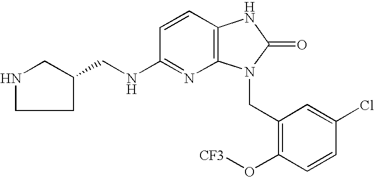 | 1 |
| 183 | 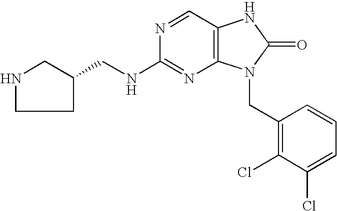 | 1 |
| 184 | 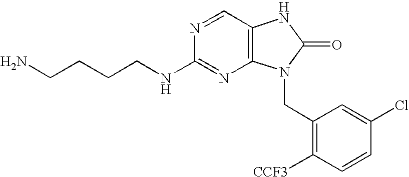 | 1 |
| 185 | 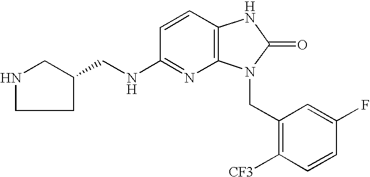 | 1 |
| 186 | 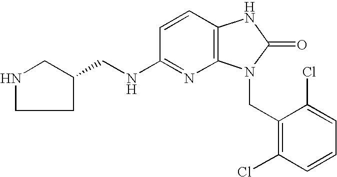 | 1 |
| 187 | 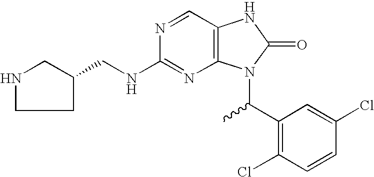 | 1 |
| 188 | 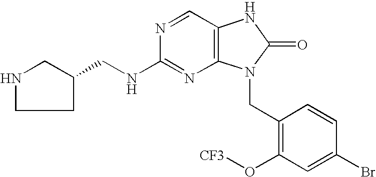 | 1 |
| 189 | 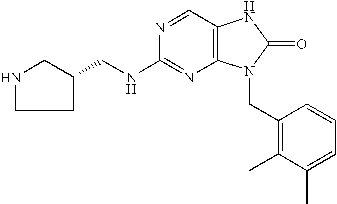 | 1 |
| 190 | 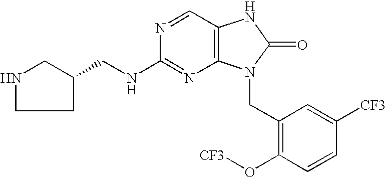 | 1 |
| 191 | 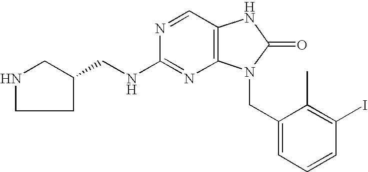 | 1 |
| 192 | 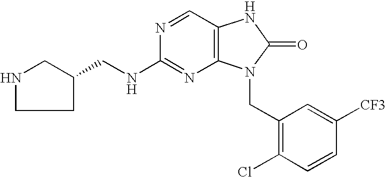 | 1 |
| 193 | 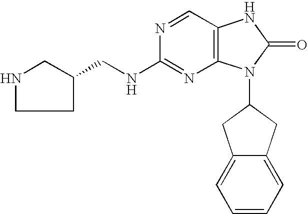 | 1 |
| 194 | 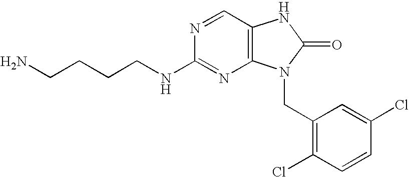 | 1 |
| 195 | 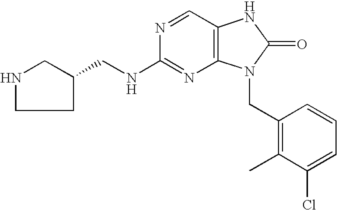 | 1 |
| 196 | 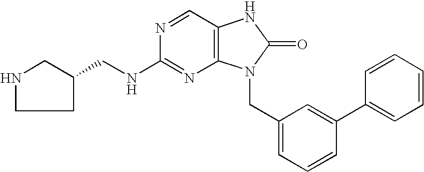 | 1 |
| 197 | 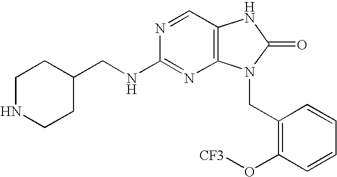 | 1 |
| 198 | 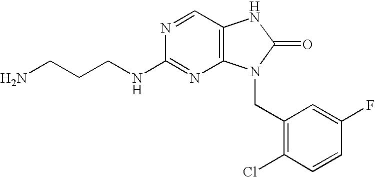 | 1 |
| 199 | 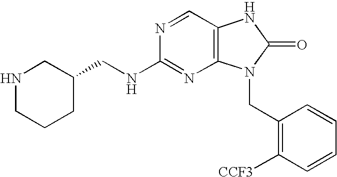 | 1 |
| 200 | 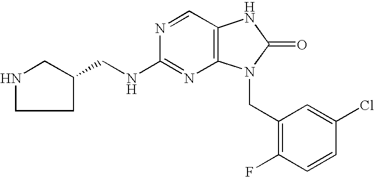 | 1 |
| 201 | 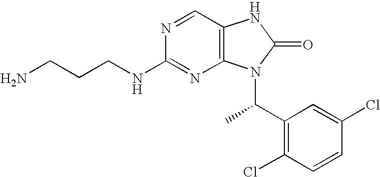 | 1 |
| 202 | 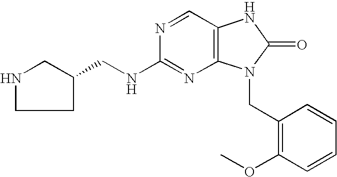 | 1 |
| 203 | 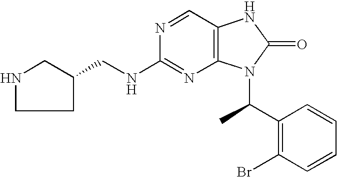 | 1 |
| 204 | 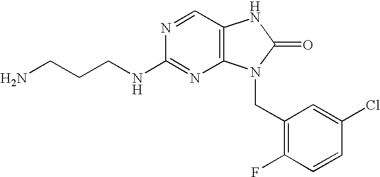 | 1 |
| 205 | 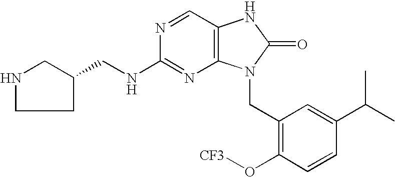 | 2 |
| 206 | 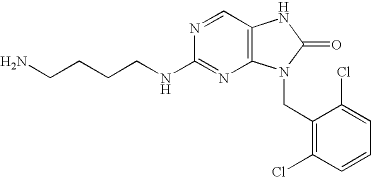 | 2 |
| 207 | 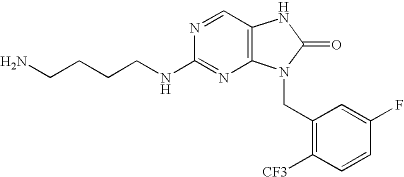 | 2 |
| 208 | 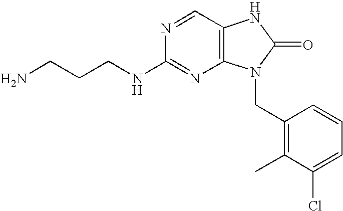 | 2 |
| 209 | 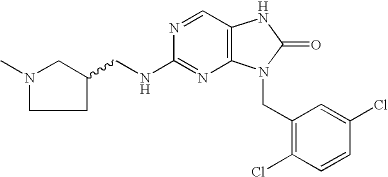 | 2 |
| 210 | 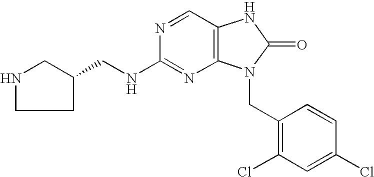 | 2 |
| 211 | 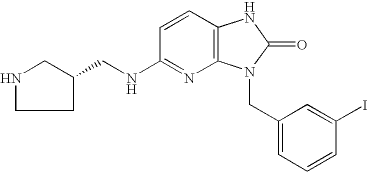 | 2 |
| 212 | 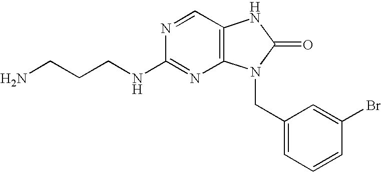 | 2 |
| 213 | 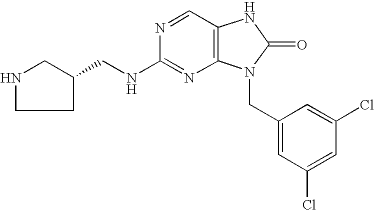 | 2 |
| 214 | 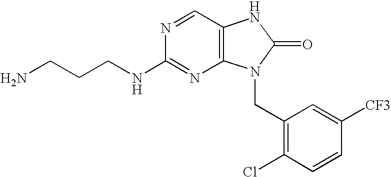 | 2 |
| 215 | 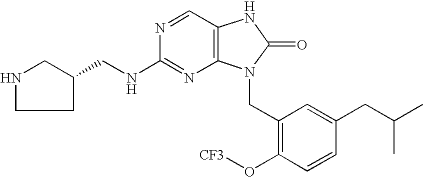 | 2 |
| 216 | 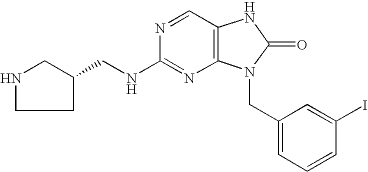 | 2 |
| 217 | 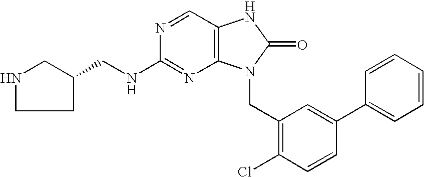 | 2 |
| 218 | 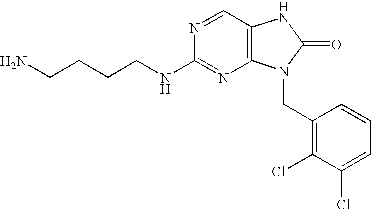 | 2 |
| 219 | 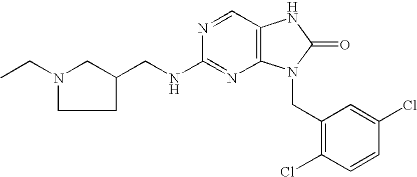 | 2 |
| 220 | 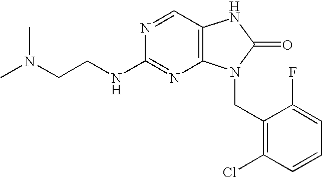 | 2 |
| 221 | 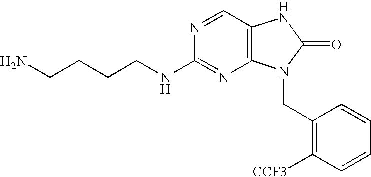 | 2 |
| 222 | 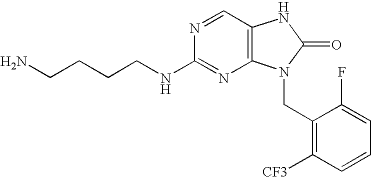 | 2 |
| 223 | 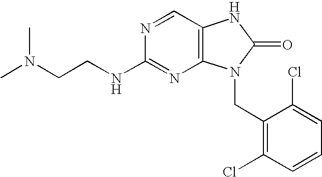 | 2 |
| 224 | 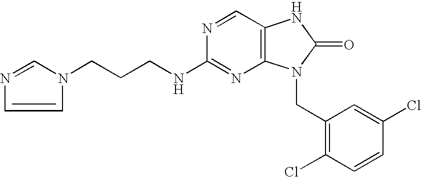 | 2 |
| 225 | 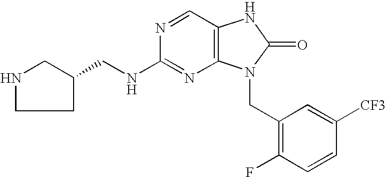 | 2 |
| 226 | 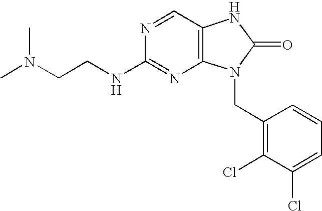 | 2 |
| 227 | 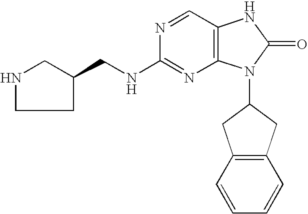 | 2 |
| 228 | 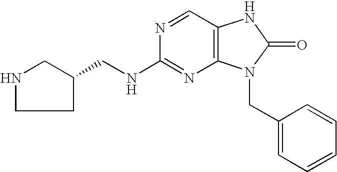 | 2 |
| 229 | 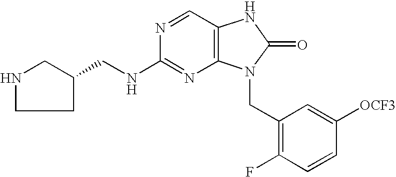 | 2 |
| 230 | 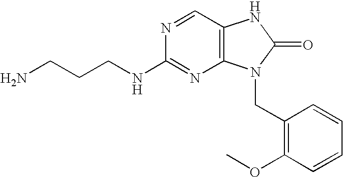 | 2 |
| 231 | 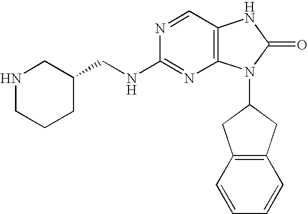 | 2 |
| 232 | 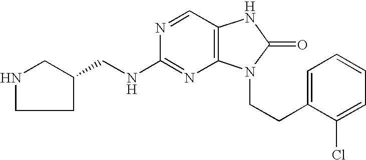 | 2 |
| 233 | 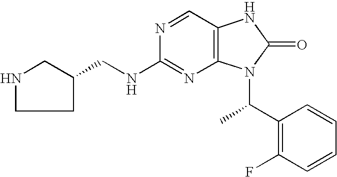 | 2 |
| 234 | 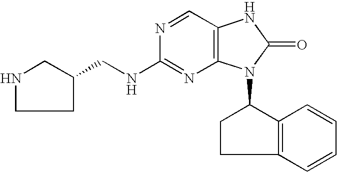 | 2 |
| 235 | 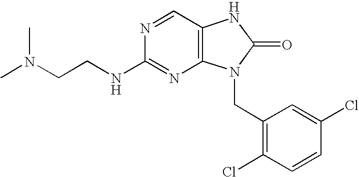 | 2 |
| 236 | 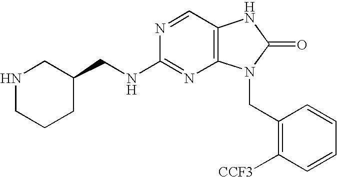 | 2 |
| 237 | 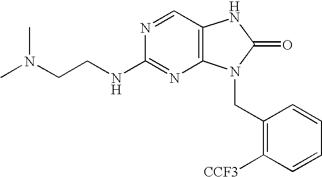 | 2 |
| 238 | 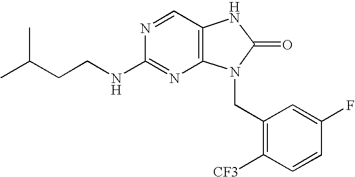 | 2 |
| 239 | 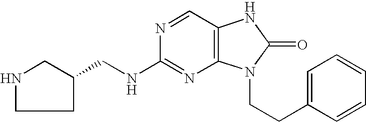 | 2 |
| 240 | 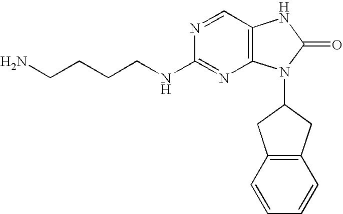 | 2 |
| 241 | 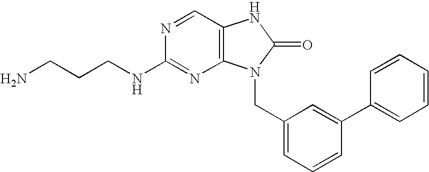 | 2 |
| 242 | 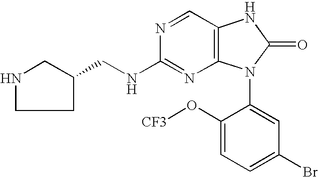 | 2 |
| 243 | 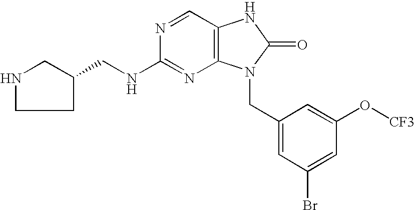 | 2 |
| 244 | 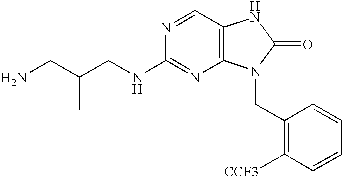 | 2 |
| 245 | 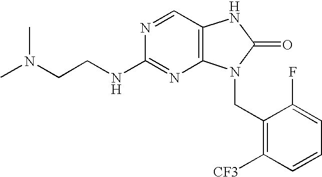 | 2 |
| 246 | 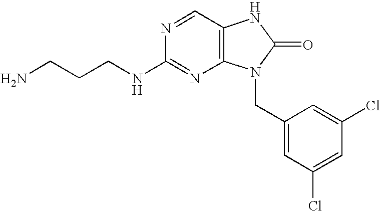 | 2 |
| 247 | 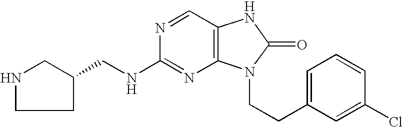 | 2 |
| 248 | 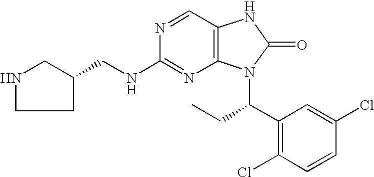 | 3 |
| 249 | 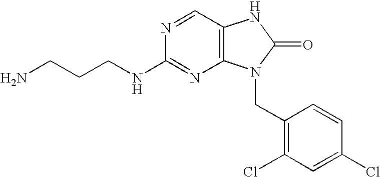 | 3 |
| 250 | 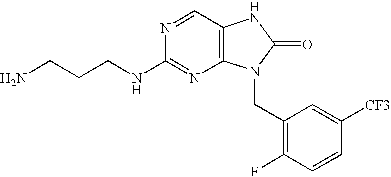 | 3 |
| 251 | 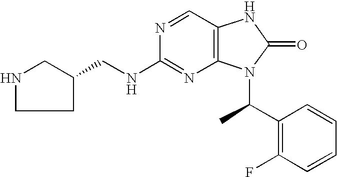 | 3 |
| 252 | 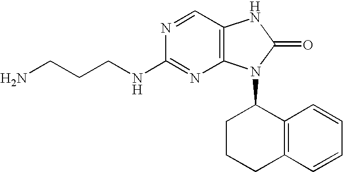 | 3 |
| 253 | 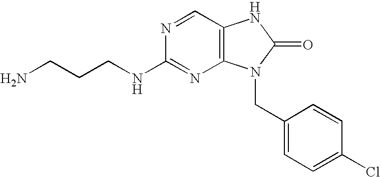 | 3 |
| 254 | 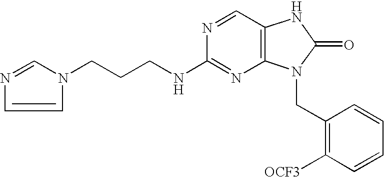 | 3 |
| 255 | 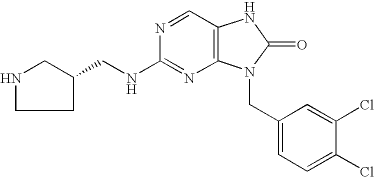 | 3 |
| 256 | 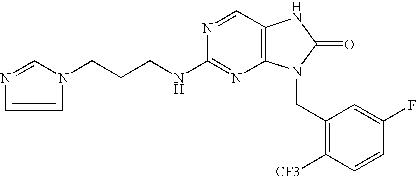 | 3 |
| 257 | 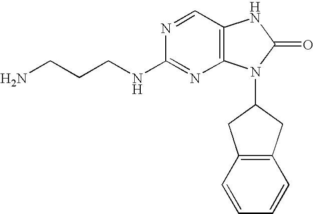 | 3 |
| 258 | 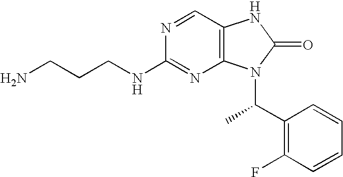 | 3 |
| 259 | 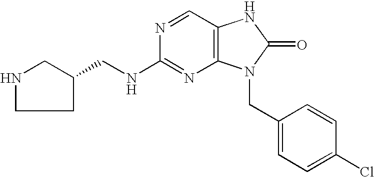 | 3 |
| 260 | 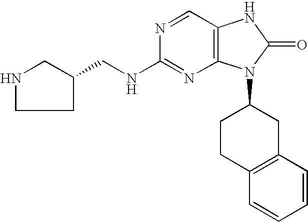 | 3 |
| 261 | 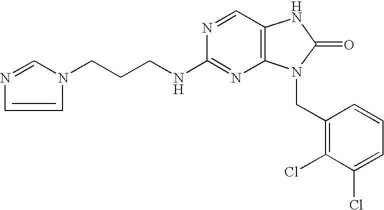 | 3 |
| 262 | 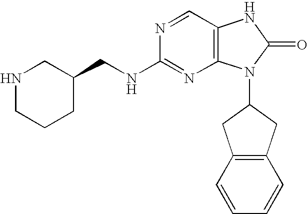 | 3 |
| 263 | 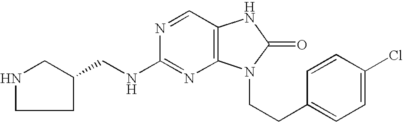 | 3 |
| 264 | 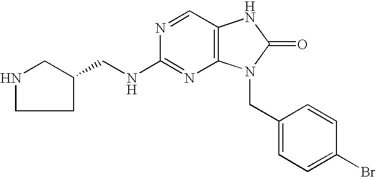 | 3 |
| 265 | 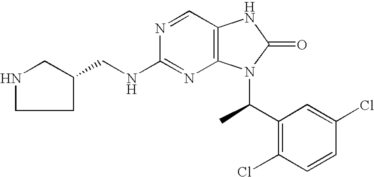 | 3 |
| 266 | 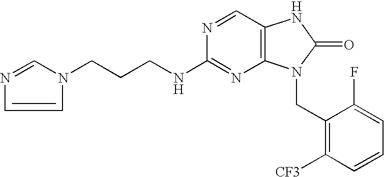 | 3 |
| 267 | 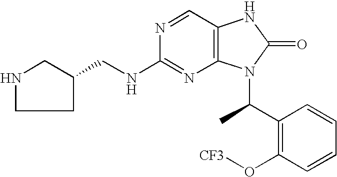 | 3 |
| 268 | 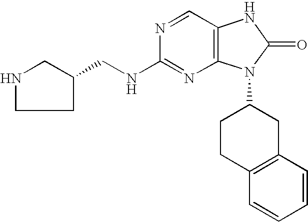 | 3 |
| 269 | 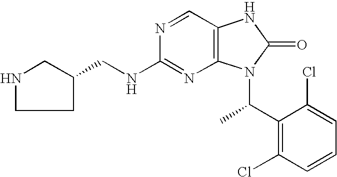 | 3 |
| 270 | 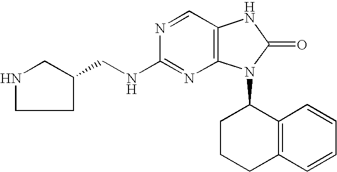 | 3 |
| 271 | 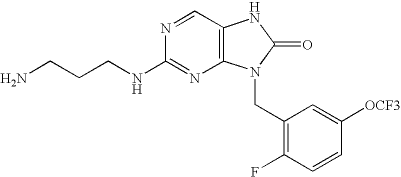 | 3 |
| 272 | 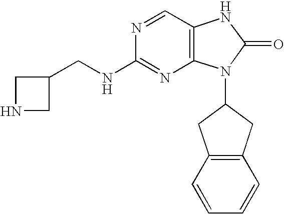 | 3 |
| 273 | 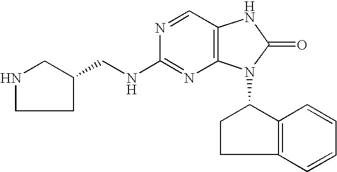 | 3 |
| 274 | 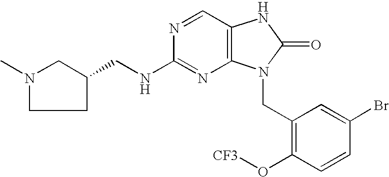 | Not Tested |

We claim:

1. A compound represented by Formula I, 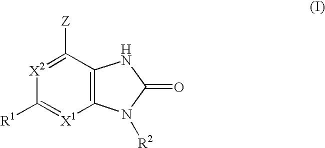
wherein:
X¹ and X² are independently chosen from N and CH, with the proviso that both
X¹ and X² are not CH;
R¹ is chosen from 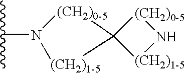
and —NHR⁴;
R⁴ is chosen from
-M-NR⁷R⁸ and 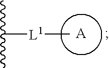
M is C₂-C₁₀ alkyl optionally substituted with —OH, with a proviso that —OH cannot be bonded to a carbon atom that is also bonded to N;
R⁷ and R⁸ are independently chosen from —H and C₁-C₄ alkyl;
A is chosen from carbocycle, substituted carbocycle, heterocycle and substituted heterocycle; and
L¹ is a C₁-C₁₀ alkyl optionally substituted with —OH, with the proviso that —OH cannot be bonded to a carbon atom that is also bonded to N;
R² is chosen from aryl, substituted aryl, arylalkyl, substituted arylalkyl, heteroaryl, substituted heteroaryl, hetroarylalkyl, and substituted heteroarylalkyl; and
Z is chosen from hydrogen, C₁-C₆ alkyl, cyano, hydroxyalkyl, carboxamido and haloalkyl;
with the provisos that, when Z is carboxamido, only one of X¹ or X² can be nitrogen; and that
8H-Purin-8-one, 2-[[2-(2-fluorophenyl)ethyl]amino]-7,9-dihydro-9-(3-methoxyphenyl) and 8H-Purin-8-one, 9-[(2,6-difluorophenyl)methyl]-2-[[(2,4-dimethoxyphenyl)methyl]amino]-7,9-dihydro are excluded compounds.

2. A compound according to claim 1, wherein Z is hydrogen.

3. A compound according to claim 2, wherein R⁴ is chosen from: 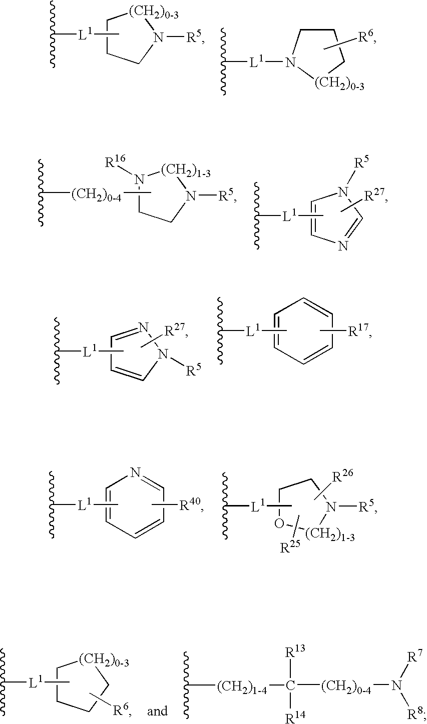
wherein
R⁵ represents one, two or three residues independently chosen from —H, C₁-C₄ alkyl, acyl, haloalkyl, hydroxyalkyl, hydroxyaminoalkyl, alkoxyalkyl and aminoalkyl;
R⁶ represents one, two or three residues independently chosen from —H, C₁-C₄ alkyl, —OCH₃, hydroxy, amino, haloalkyl, acylamino, hydroxyalkyl, hydroxyaminoalkyl, hydroxyalkylamino, alkylamino, dialkylamino and aminoalkyl;
R⁷ and R⁸ are independently chosen from —H and C₁-C₄ alkyl;
R¹³ and R¹⁴ are independently chosen from —H, —OH, and C₁-C₄ alkyl, with a proviso that —OH cannot be bonded to a carbon atom that is also bonded to N;
R¹⁶ is chosen from —H, C₁-C₄ alkyl, amino, and aminoalkyl;
R¹⁷ is chosen from C₁-C₄ alkyl, hydroxyalkyl, amino, aminoalkyl and optionally substituted heterocycloalkyl;
R⁴⁰ is chosen from —H, amino and lower alkyl;
R²⁵ is chosen from —H and lower alkyl;
R²⁶ is chosen from —H and oxo; and
R²⁷ is chosen from —H, C₁-C₄ alkyl, amino and alkylamino.

4. A compound according to claim 3 wherein L¹ is —CH₂.

5. A compound according to claim 4 wherein R⁴ is

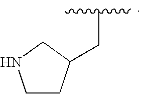

6. A compound according to claim 1 wherein R⁴ is -M-NR⁷R⁸.

7. A compound according to claim 6 wherein M is C₂-C₄ alkyl optionally substituted with —OH.

8. A compound according to claim 7 wherein R⁷ and R⁸ are each independently chosen from hydrogen and methyl.

9. A compound according to claim 1 wherein X¹ and X² are both nitrogen.

10. A compound according to claim 1 wherein X¹ is nitrogen and X² is CH.

11. A compound according to claim 1 wherein X¹ is CH and X² is nitrogen.

12. A compound according to claim 1 wherein R² is chosen from 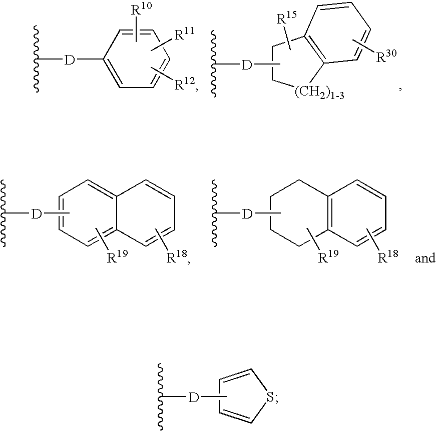
wherein
R¹⁰, R¹¹, and R¹² are independently chosen from —H, halogen,
—OCH₃, —OCF₃, —CH₂OCF₃, —CF₃, —CN, alkylthio, —SO₂Me, —SCF₃, C₁-C₆ alkyl, substituted C₁-C₆ alkyl, C₁-C₄ alkenyl, C₁-C₄ alkynyl, C₁-C₄ acetate, C₁-C₄ alkylamino, carboxamide, substituted carboxamide, aryl, substituted aryl, heterocycle, substituted heterocycle, aryloxy, and substituted aryloxy;
R¹⁵ is chosen from hydroxyl and alkoxy;
R¹⁸ and R¹⁹ are each independently chosen from hydrogen, halogen, C₁-C₄ alkyl, cyano, C₁-C₄ alkynyl and trifluoromethoxy;
R³⁰ represents one or two residues independently chosen from hydrogen and halogen; and
D is C₀-C₆ alkyl.

13. A compound according to claim 12 of formula

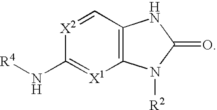

14. A compound according to claim 13 wherein R² is 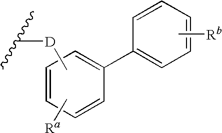
wherein
R^{a} represents one, two, or three residues independently chosen from —H, halogen, —OCH₃, —OCF₃, —CF₃, —CN, —SMe, —SO₂Me, —SCF₃, C₁-C₄ alkyl, amino, and aminoalkyl; and
R^{b} represents one, two, or three residues independently chosen from halogen, —OCH₃, —OCF₃, —CF₃, —CN, —SMe, —SO₂Me, —SCF₃, C₁-C₄ alkyl, carboxamide, alkoxyalkyl, amino, and aminoalkyl.

15. A compound according to claim 14 wherein D is C₀-C₂ alkyl.

16. A compound according to claim 15 wherein D is —CH₂.

17. A compound according to claim 12 wherein R² is 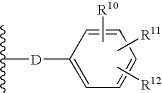
and D is C₀-C₃ alkyl.

18. A compound according to claim 13 wherein R² is 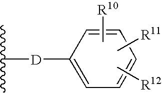
and D is C₀-C₃ alkyl.

19. A compound according to claim 18 wherein R² is

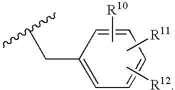

20. A compound according to claim 13 wherein R² is

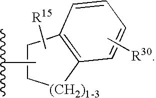

21. A compound according to claim 13 wherein R² is chosen from 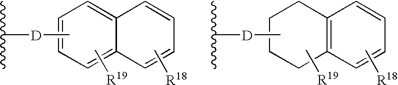
and D is C₀-C₃ alkyl.

22. A compound according to claim 13, wherein:
R⁴ is chosen from 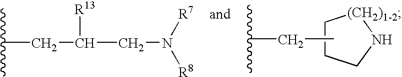
wherein
R⁷ and R⁸ are independently chosen from —H and —CH₃; and R¹³ is chosen from —H and —OH.

23. A compound according to claim 22, wherein:
R⁴ is chosen from 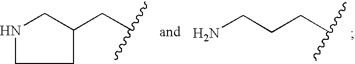
and R² is 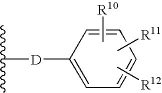
wherein
D is a C₁-C₆ linear or branched alkyl; and
R¹⁰, R¹¹, and R¹² are independently chosen from —H, halogen, —OCH₃, —OCF₃, —CH₂OCF₃, —CF₃, —CN, —SMe, —SO₂Me, —SCF₃, C₁-C₆ alkyl, C₁-C₄ alkenyl, C₁-C₄ alkynyl, C₁-C₄ acetate, C₁-C₄ alkylamino, carboxamide, substituted carboxamide, aryl, substituted aryl, heterocycle, substituted heterocycle, aryloxy, and substituted aryloxy.

24. A compound according to claim 1 wherein R⁴ is chosen from

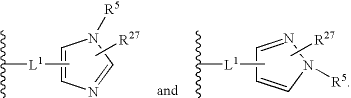

25. A compound according to claim 24 wherein R⁴ is 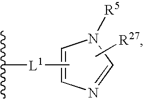
R⁵ is selected from hydrogen and C₁-C₆ alkyl and R²⁷ is C₁-C₆ alkyl.

26. A compound according to claim 13 wherein
R⁴ is 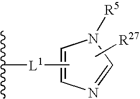
and R² is

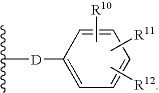

27. A compound according to claim 26 wherein
L¹ is —CH₂;
X¹ and X² are both nitrogen;
Z is hydrogen;
R⁵ is hydrogen;
R²⁷ is methyl;
R¹⁰ is chloro; and
R¹² is —OCF₃.

28. A compound according to claim 13 wherein R⁴ is 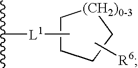
wherein
L¹ is —CH₂; and
R⁶ is chosen from hydrogen, amino, aminoalkyl, hydroxyalkylamino, hydroxyl, acylamino and hydroxyalkyl.

29. A compound according to claim 13 wherein R⁴ is 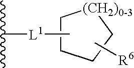
and R⁶ is chosen from amino and hydroxyl.

30. A compound according to claim 29 wherein R⁴ is selected from

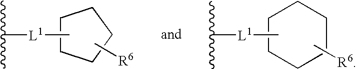

31. A compound according to claim 30 wherein L¹ is CH₂ and R² is

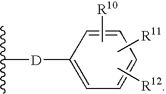

32. A compound according to claim 31 wherein R¹⁰ is —OCF₃, R¹¹ is halogen and R¹² is hydrogen.

33. A compound according to claim 14, wherein
R^{a} represents one, two, or three residues independently chosen from —H, —OCF₃ and C₁-C₄ alkyl; and
R^{b} represents one, two, or three residues independently chosen from hydrogen, halogen, C₁-C₄ alkyl, alkoxycarbonyl, carboxamide, alkoxyalkyl, amide, hydroxyalkyl and aminoalkyl.

34. A compound according to claim 33, wherein R^{a} is chosen from C₁-C₄ alkyl and hydrogen and R^{b} is chosen from halogen and aminoalkyl.

35. A compound according to claim 34, wherein R^{a} is chosen from methyl and hydrogen and R^{b} is selected from fluorine, chlorine or —CH₂NH₂.

36. A compound according to claim 1 selected from the compounds listed in Tables 1, 2 and 3 of the specification.

37. A compound according to claim 1 selected from

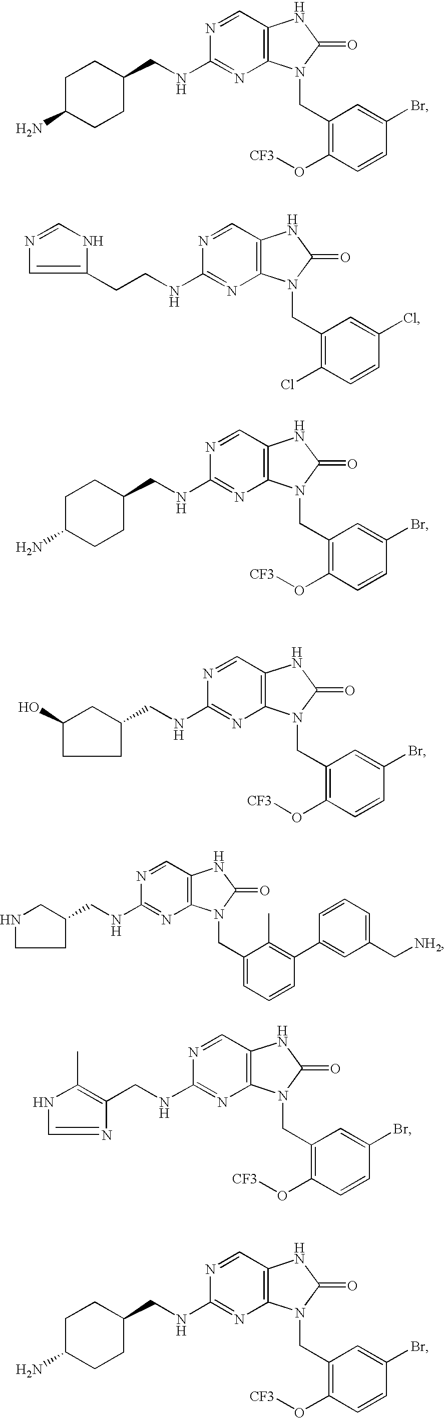

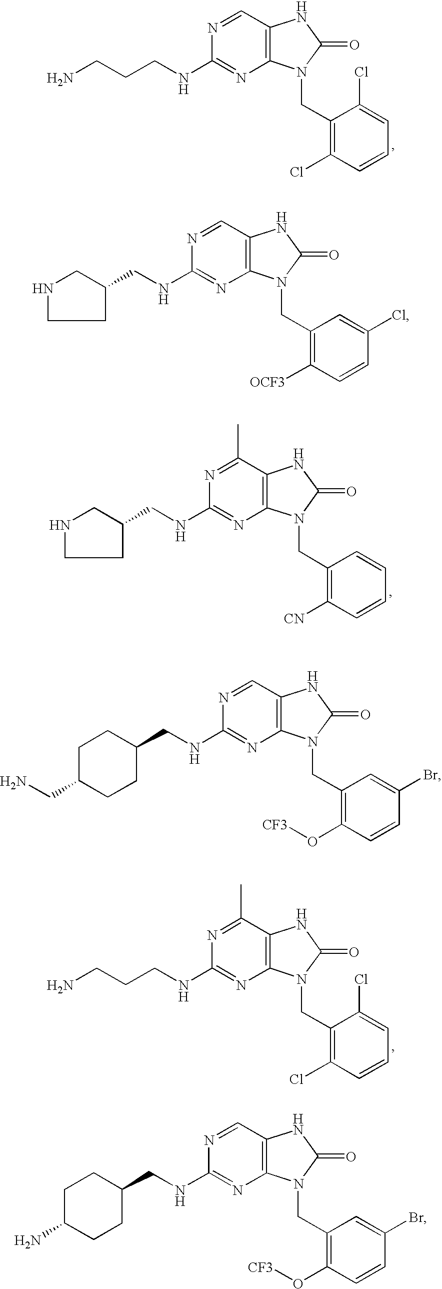

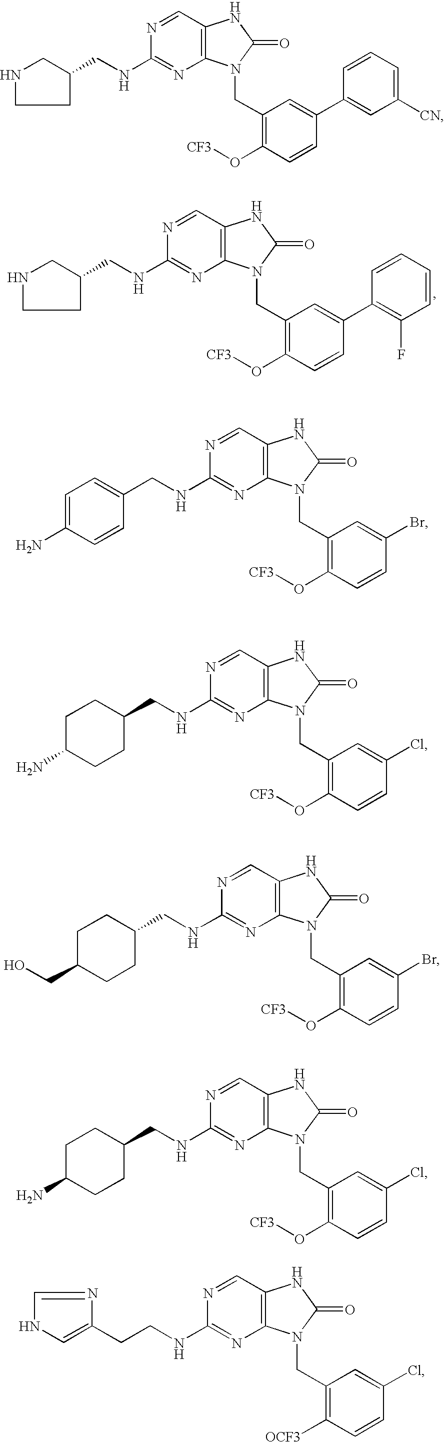

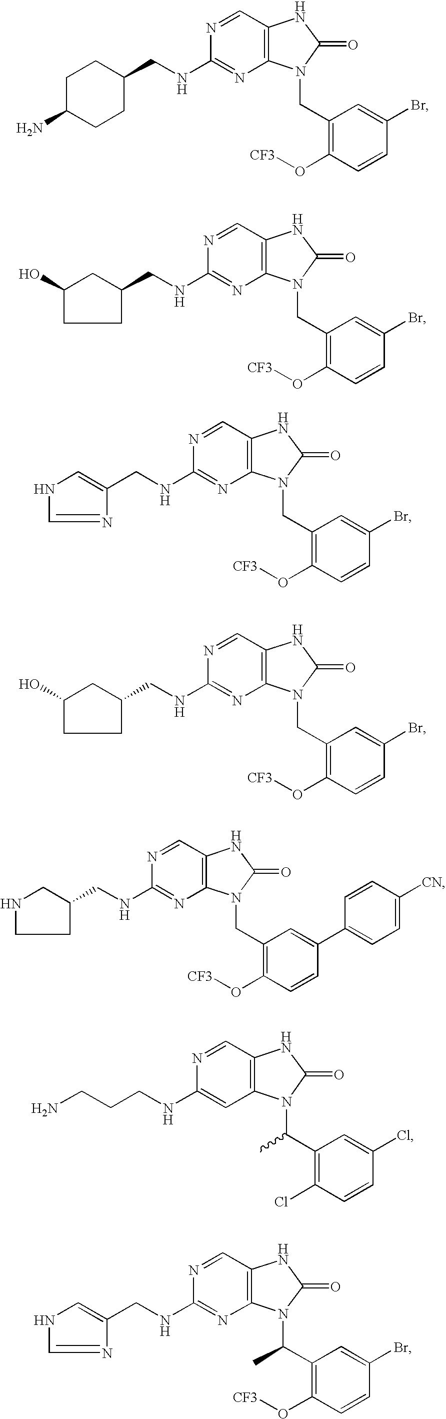

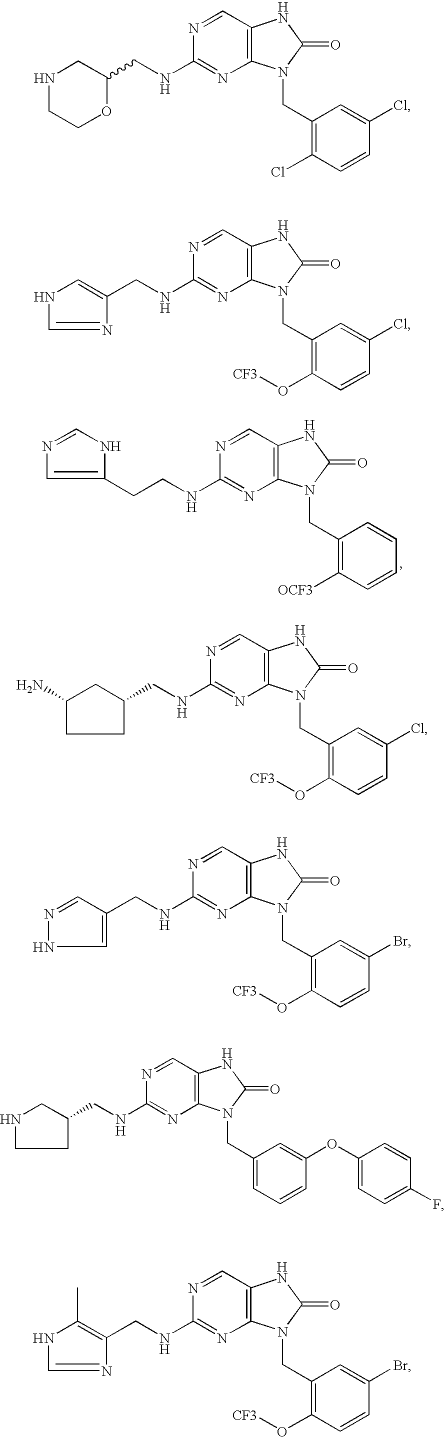

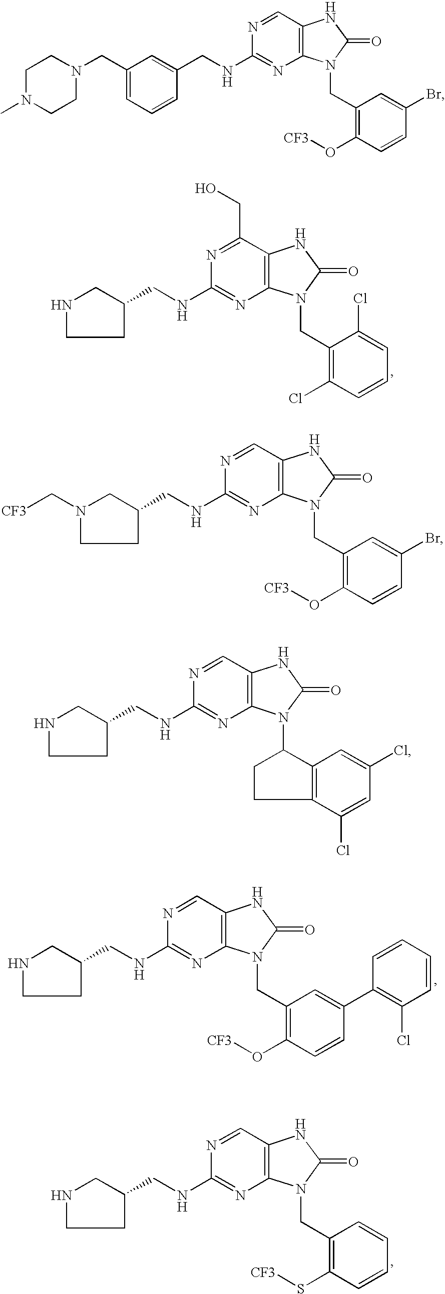

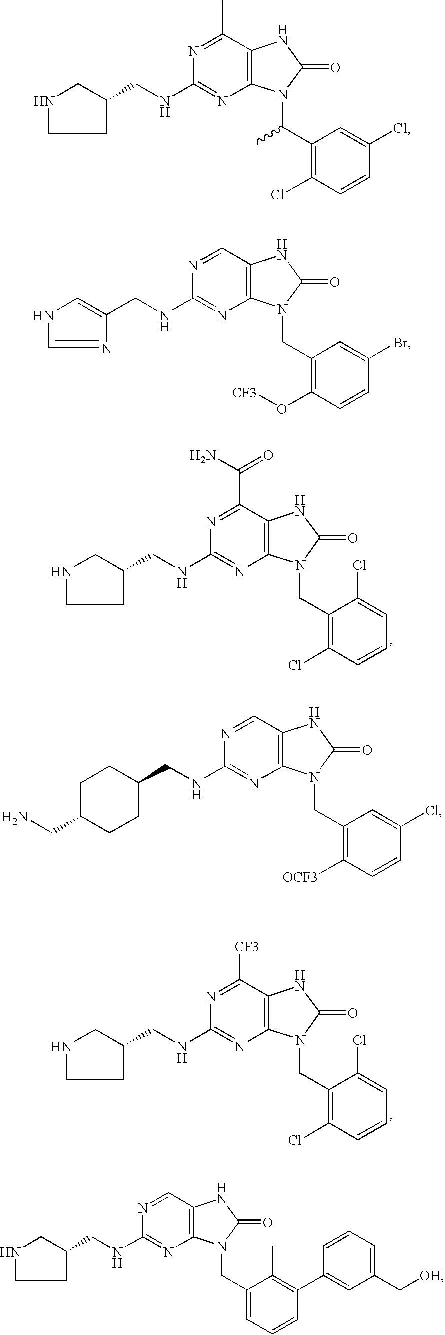

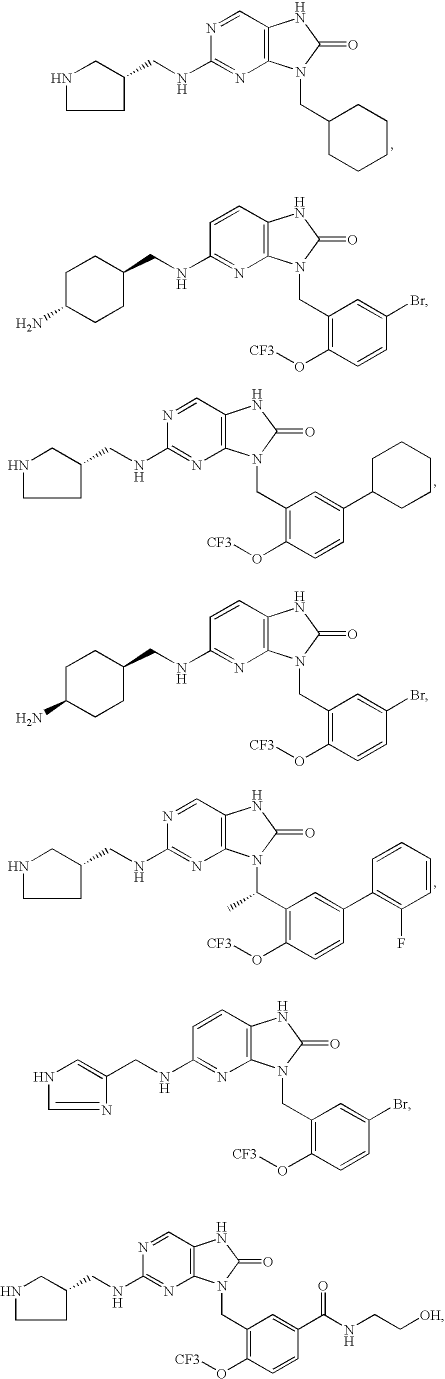

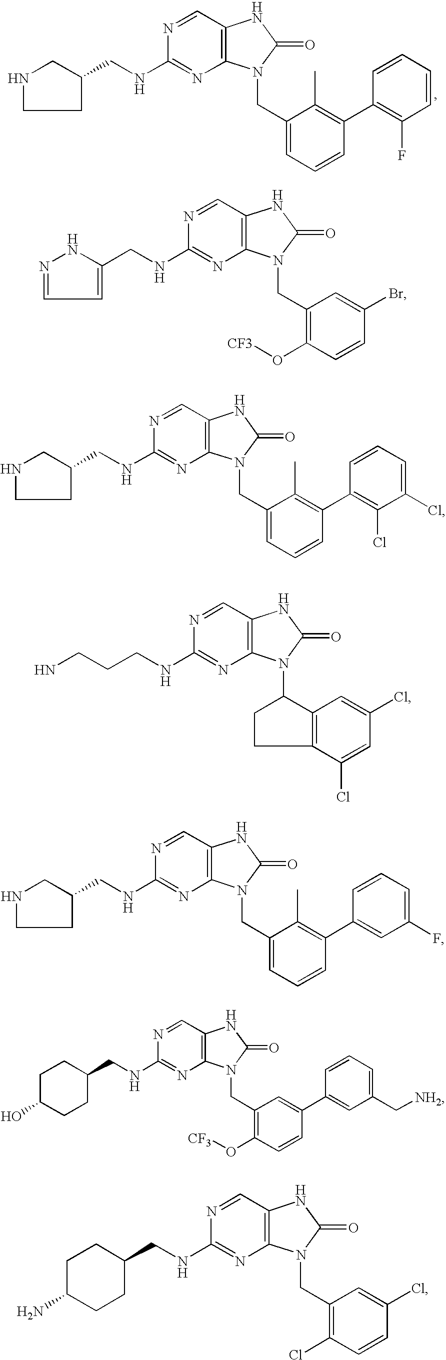

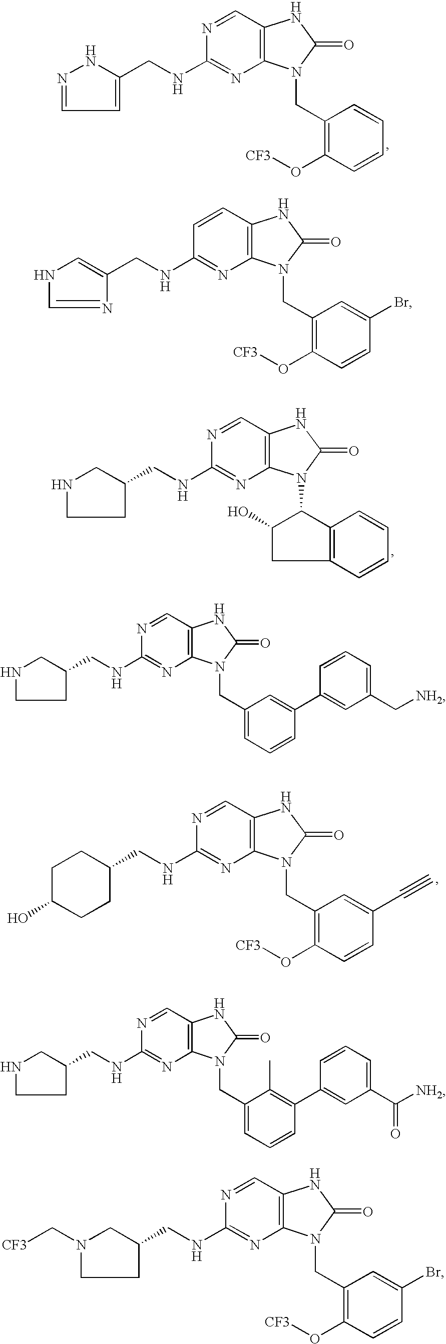

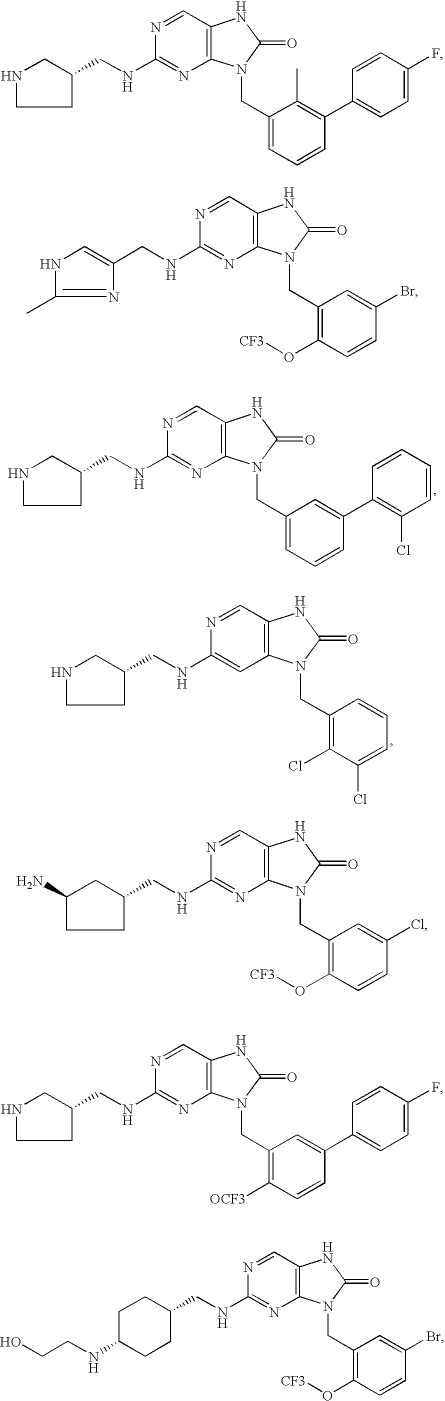

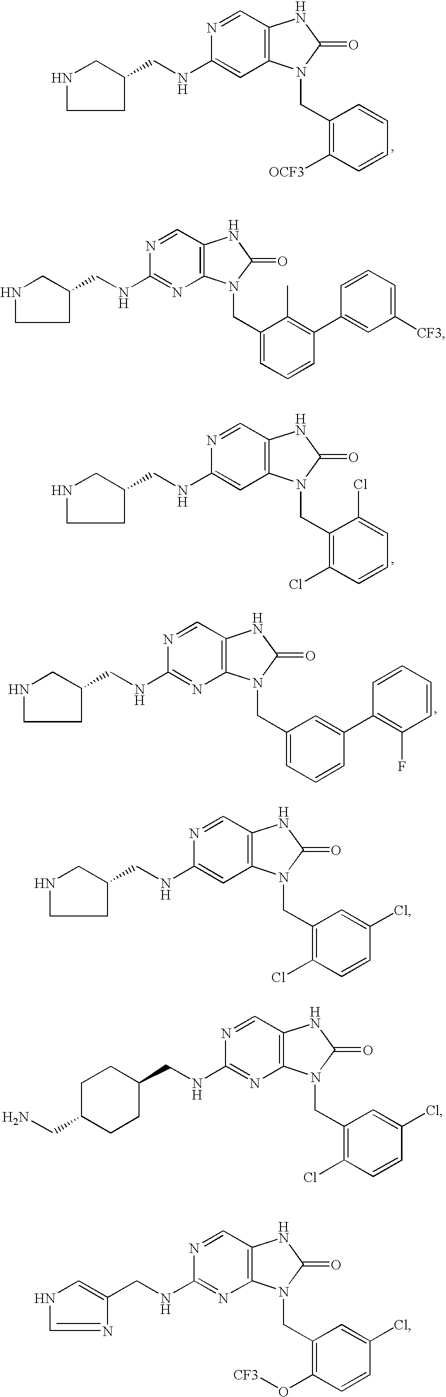

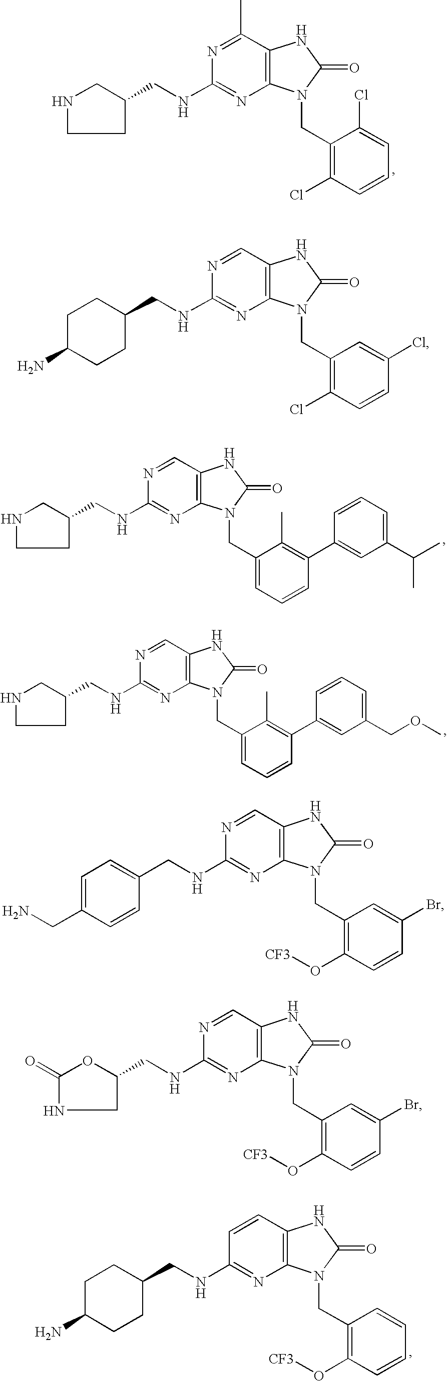

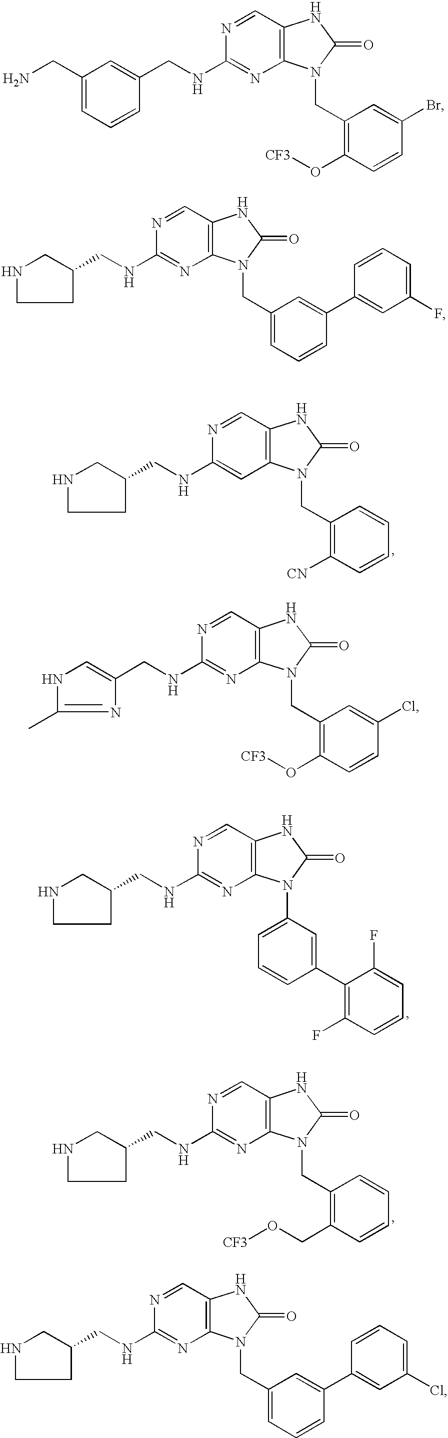

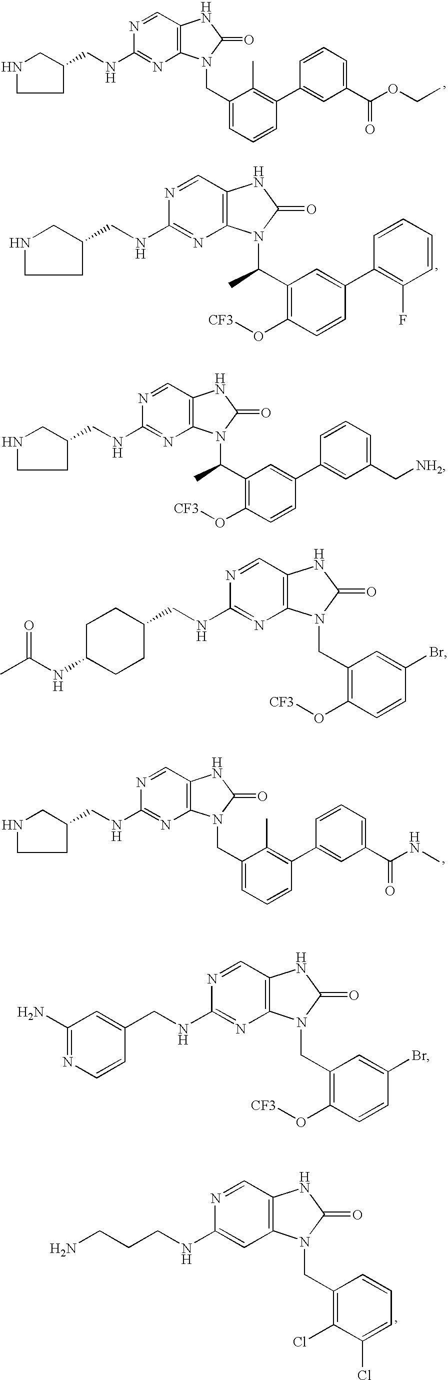

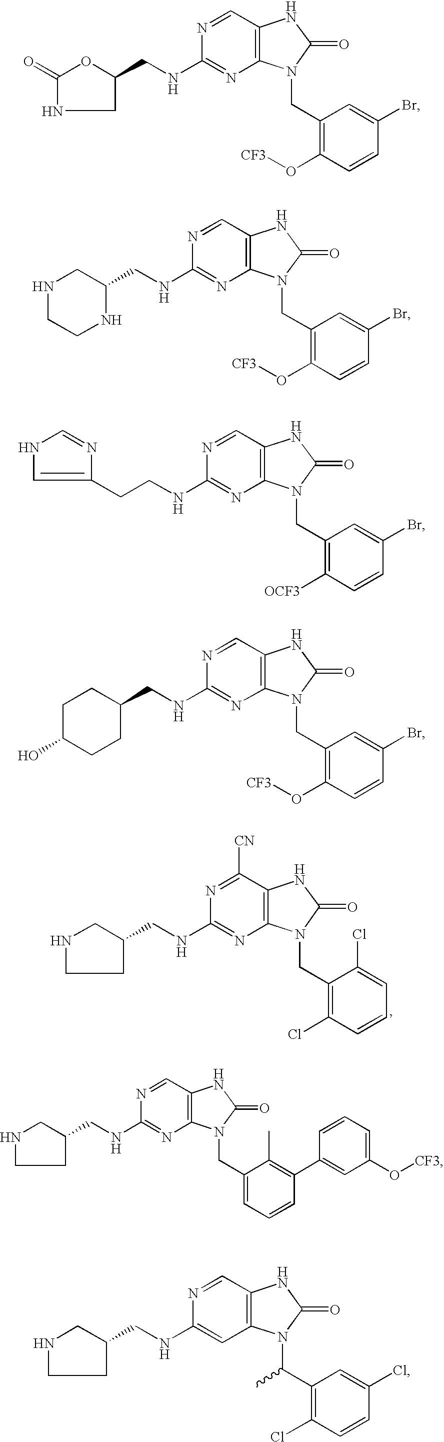

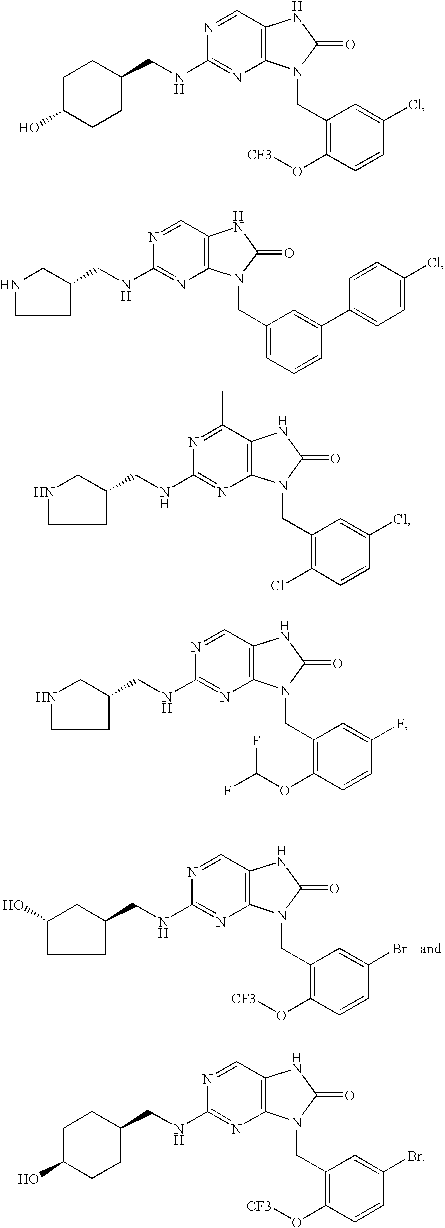

38. A compound according to claim 1 selected from

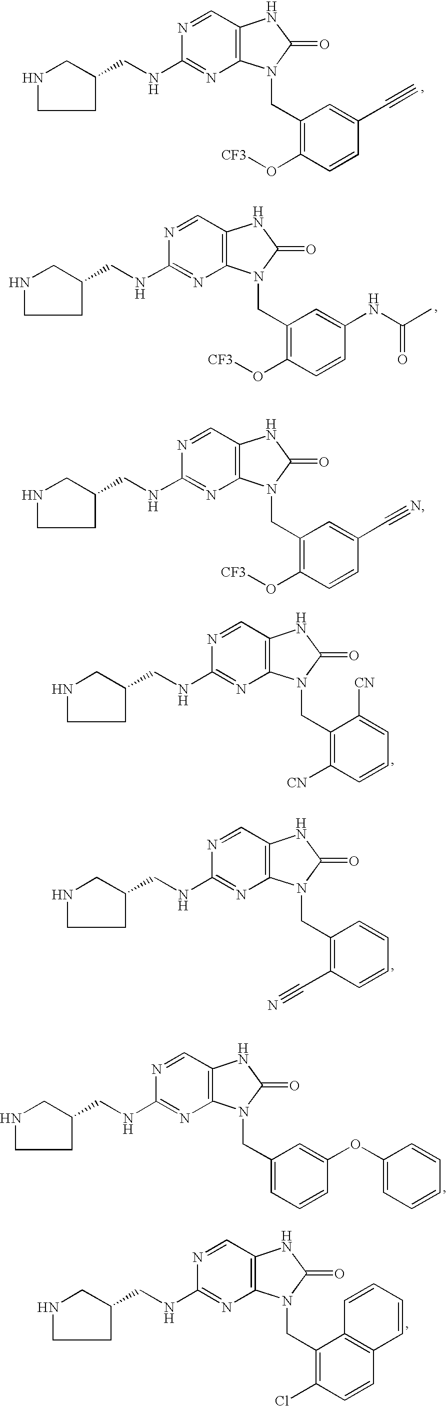

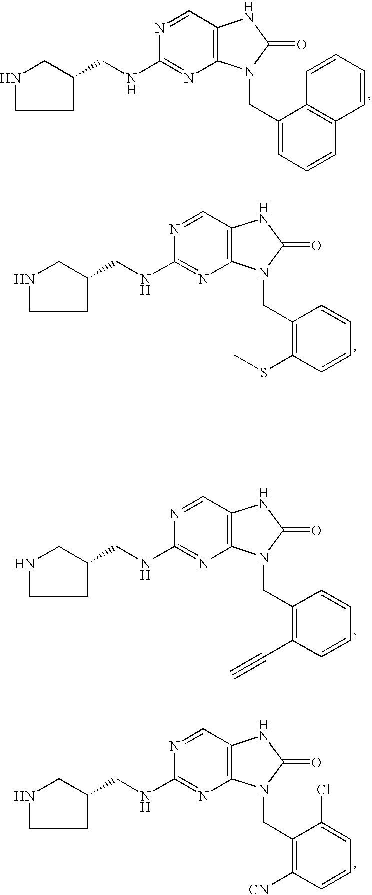

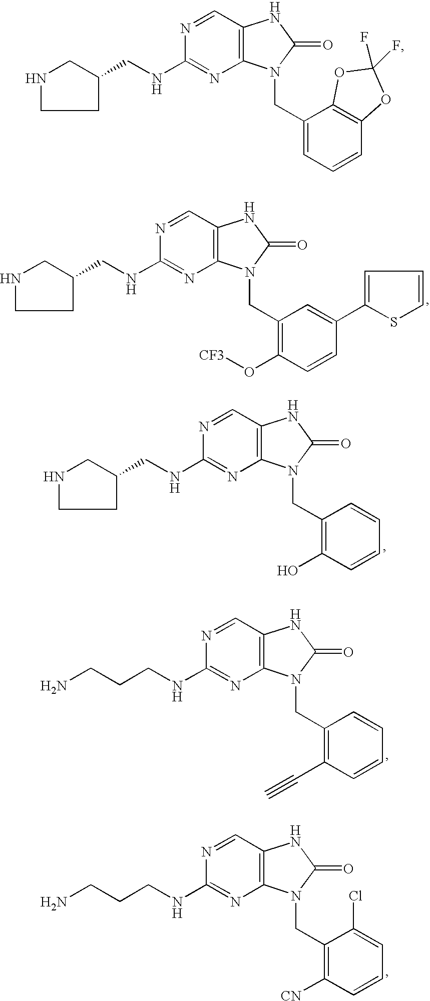

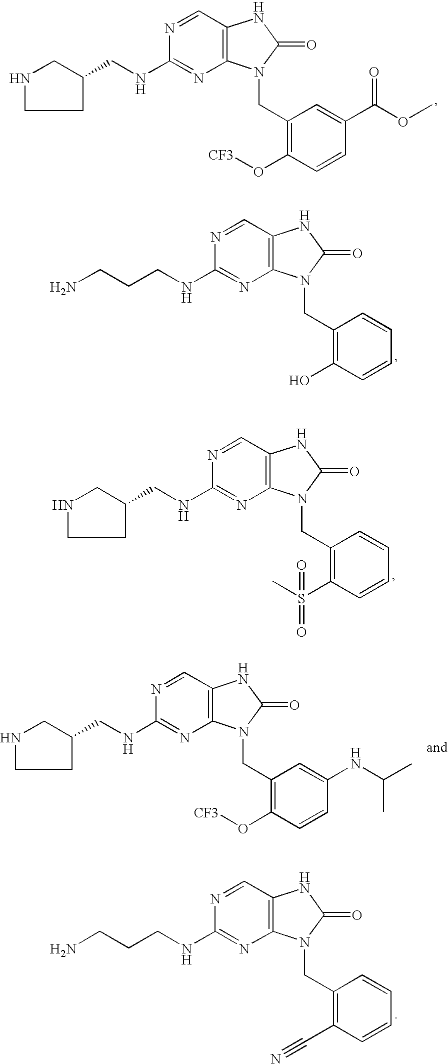

39. A compound according to claim 1 selected from

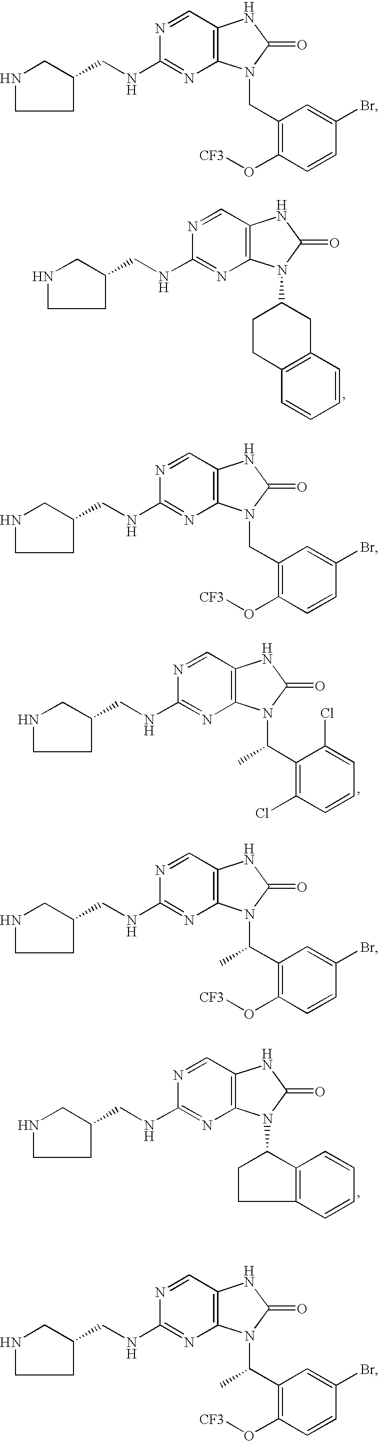

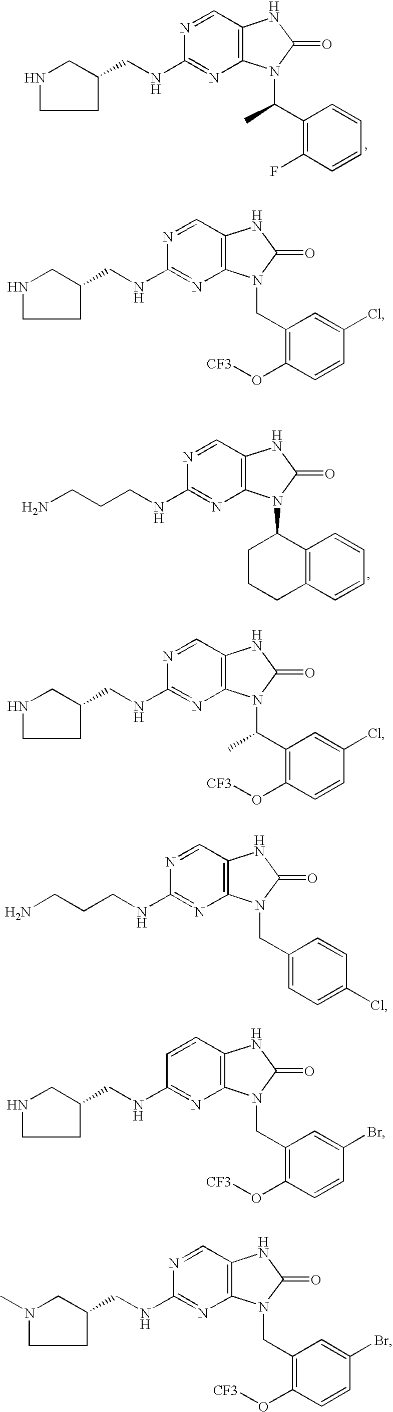

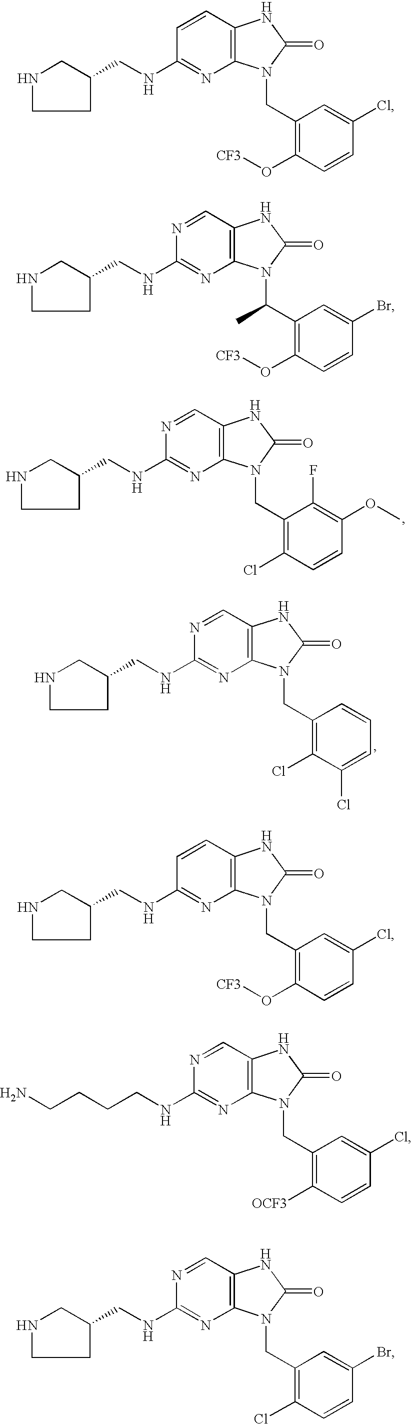

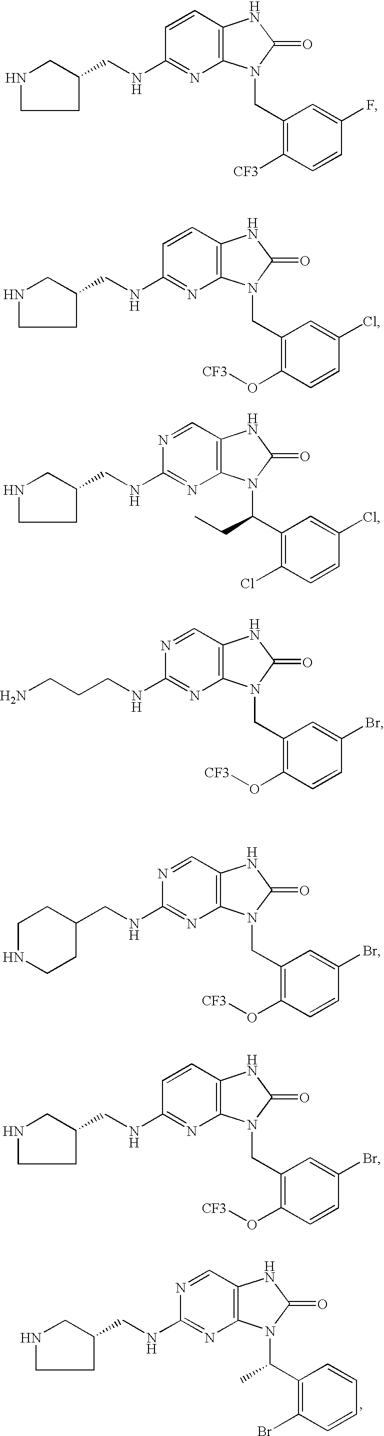

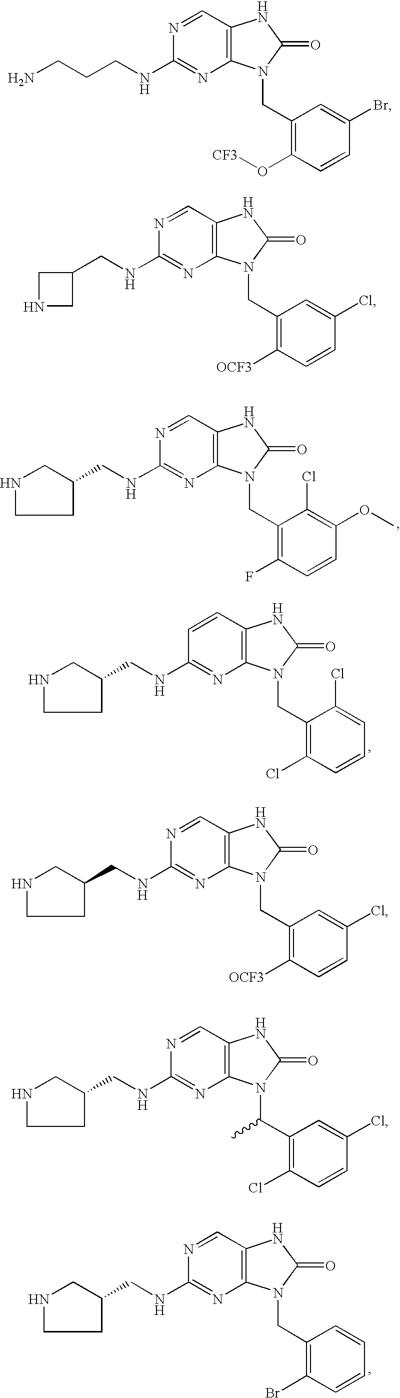

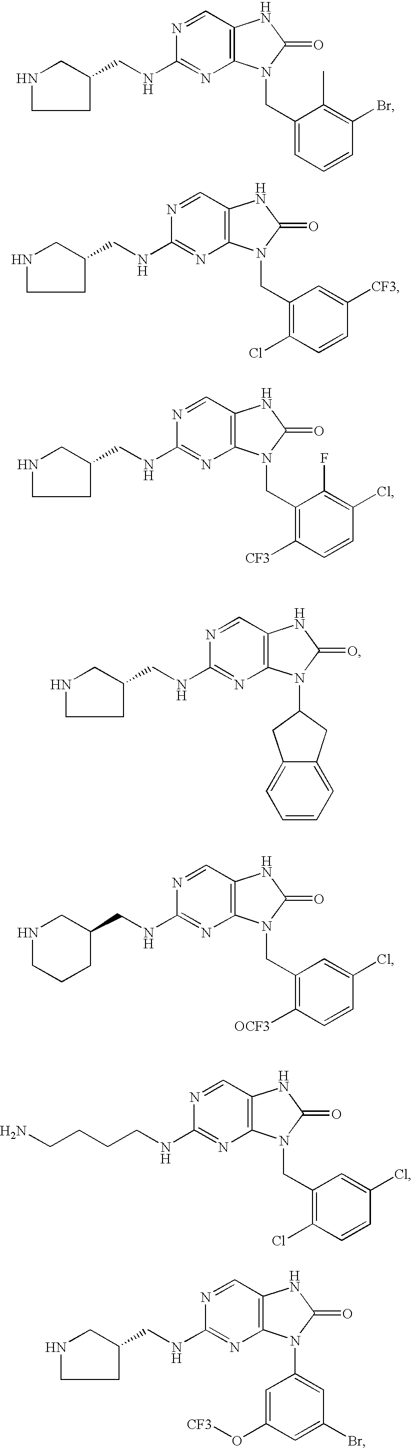

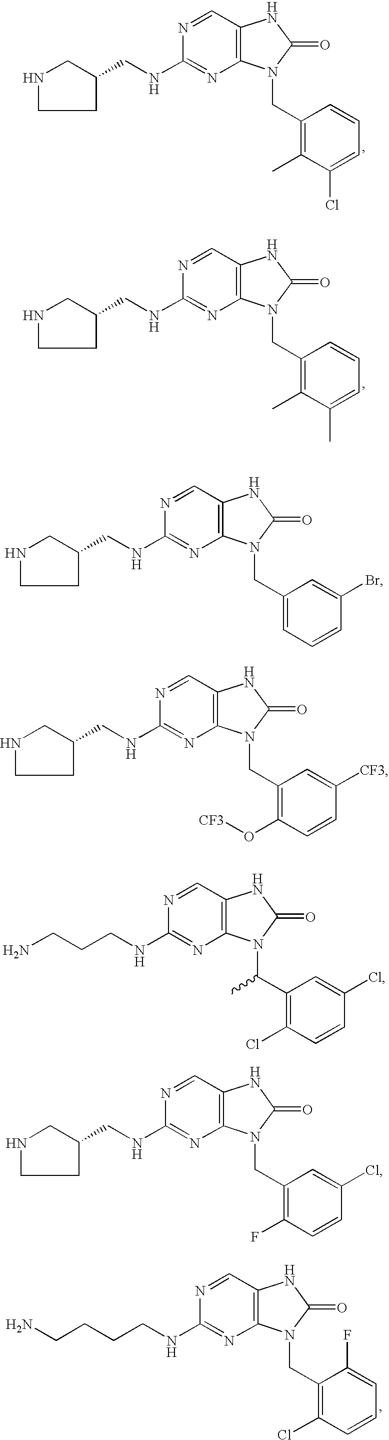

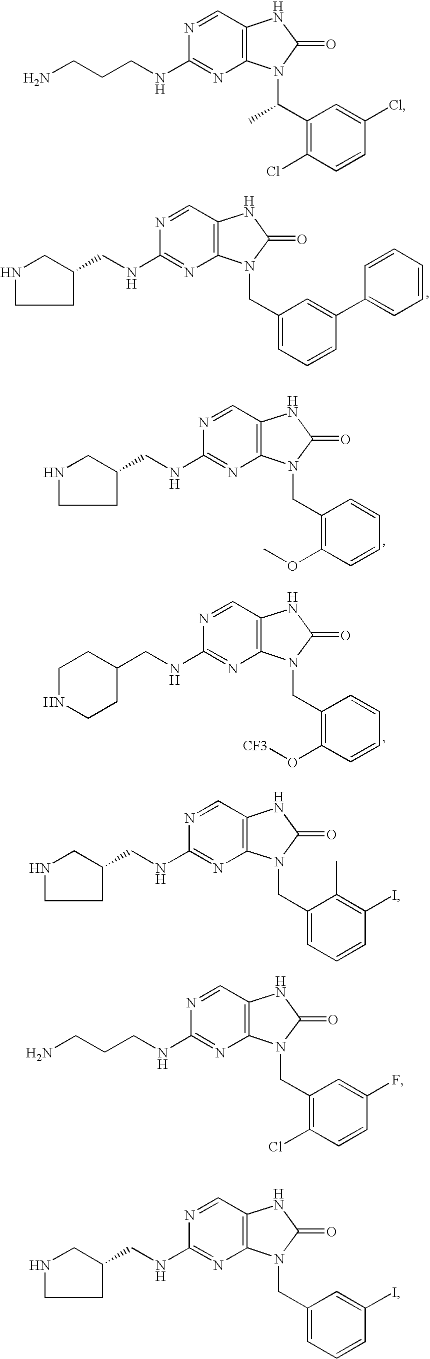

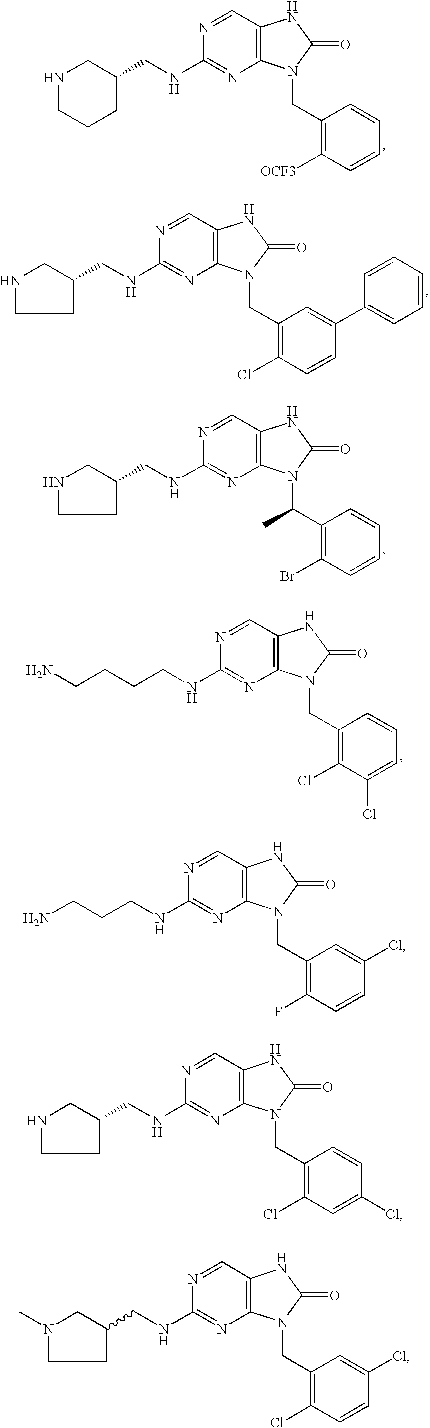

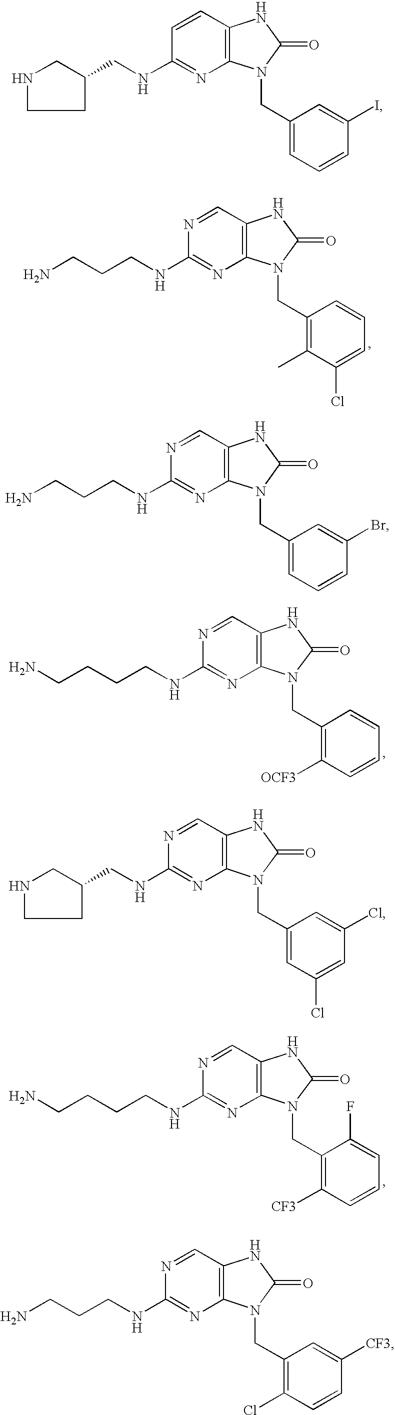

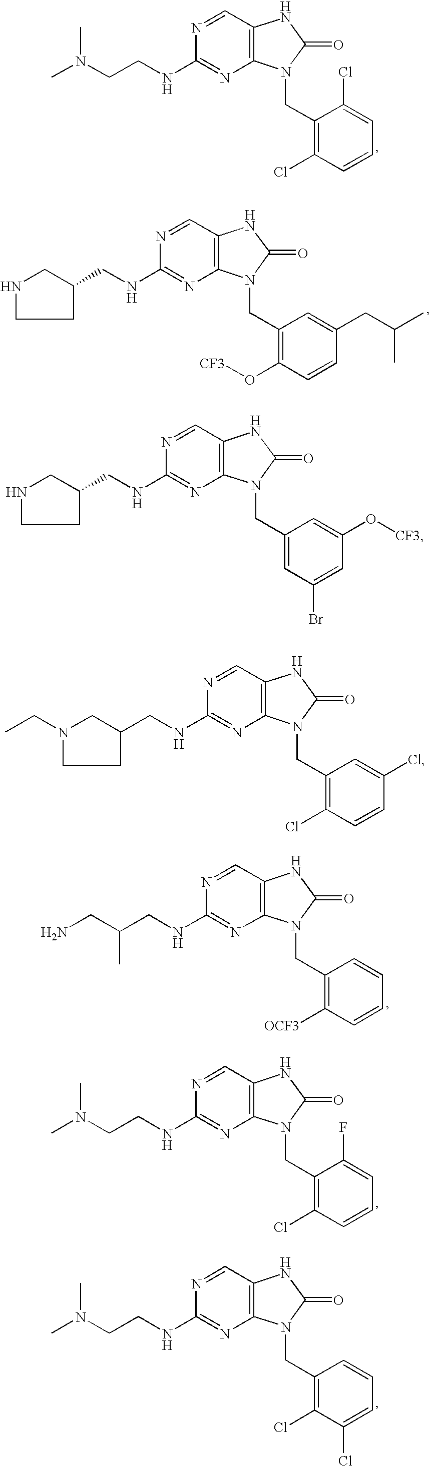

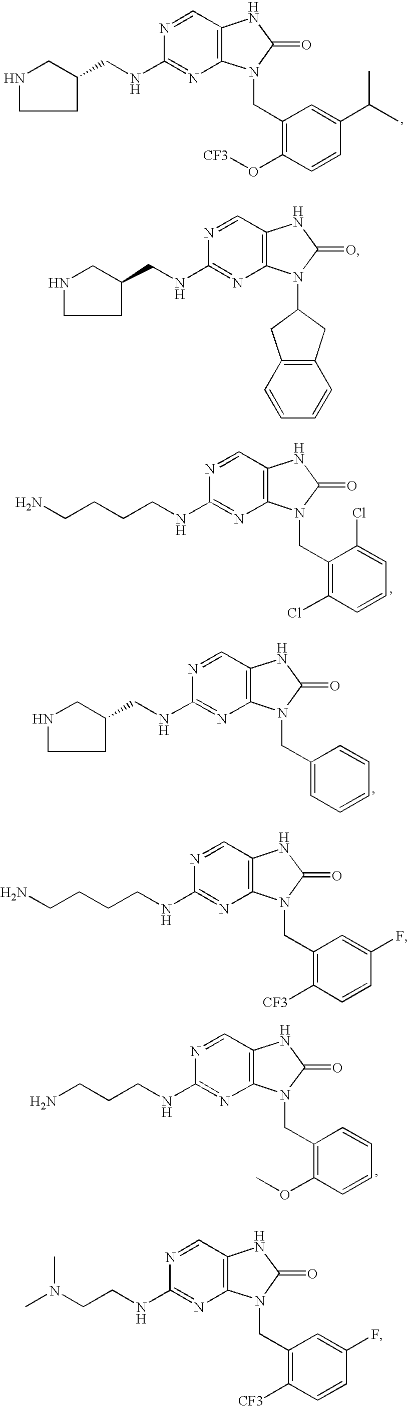

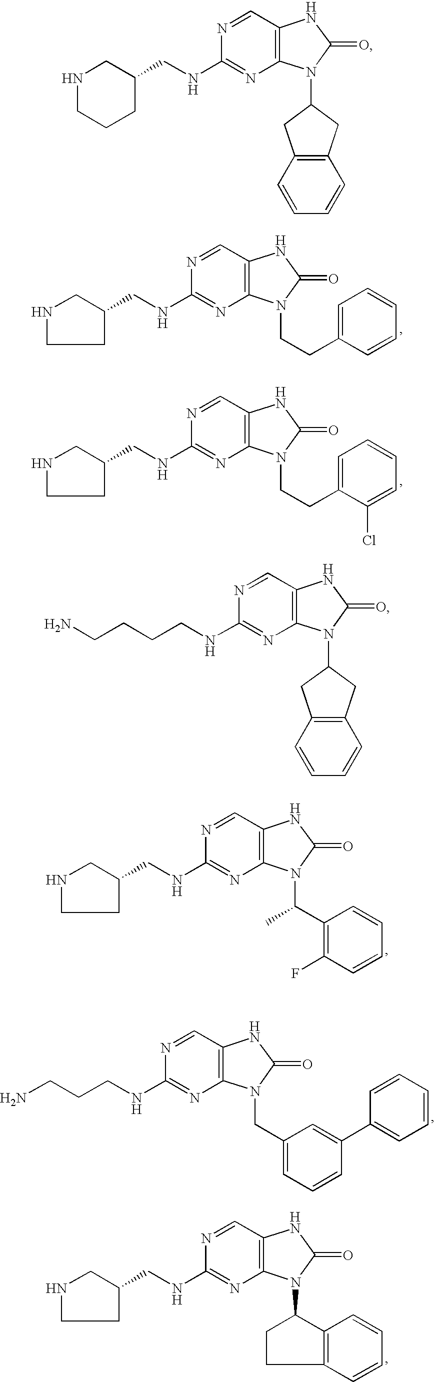

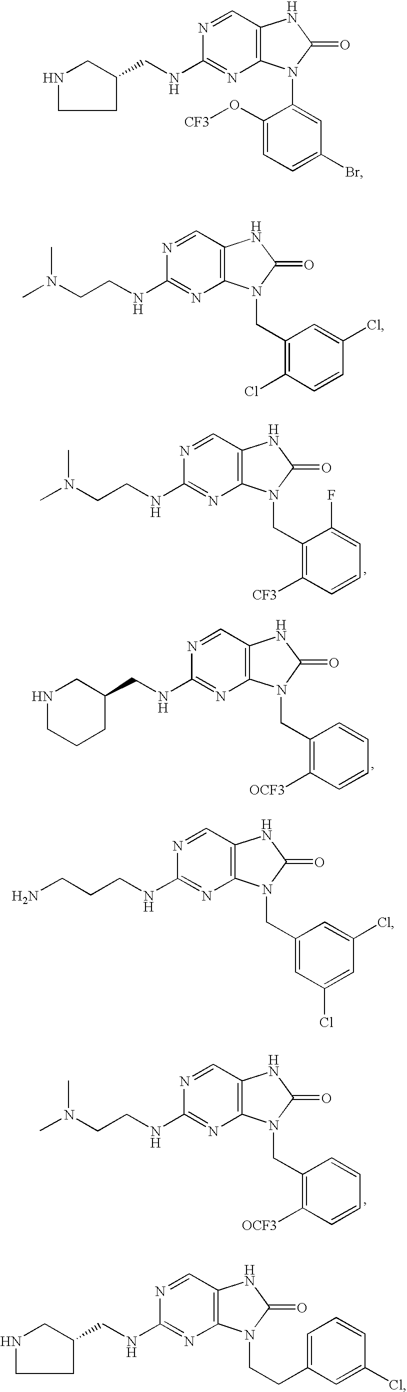

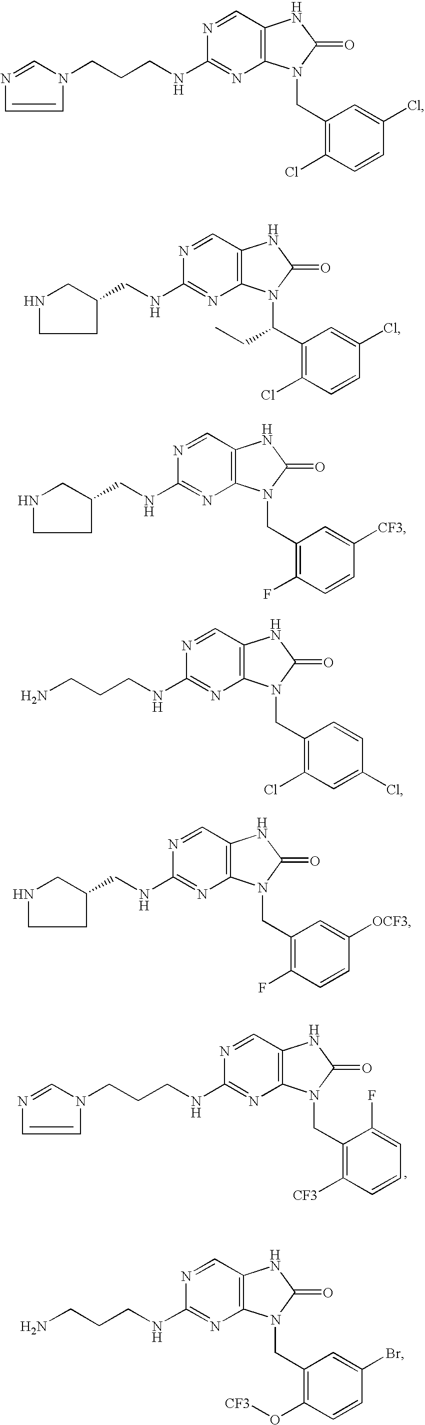

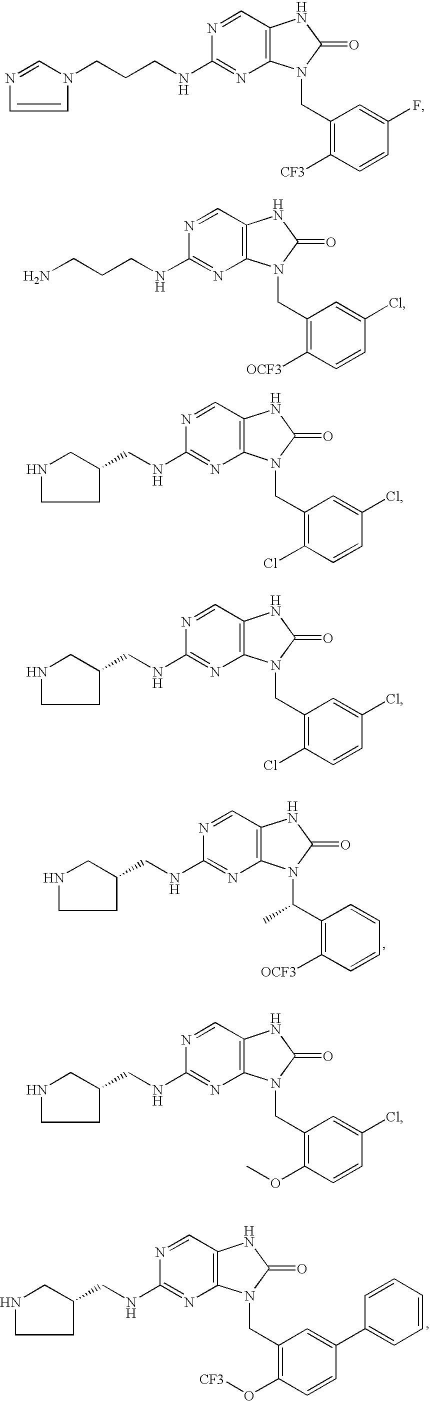

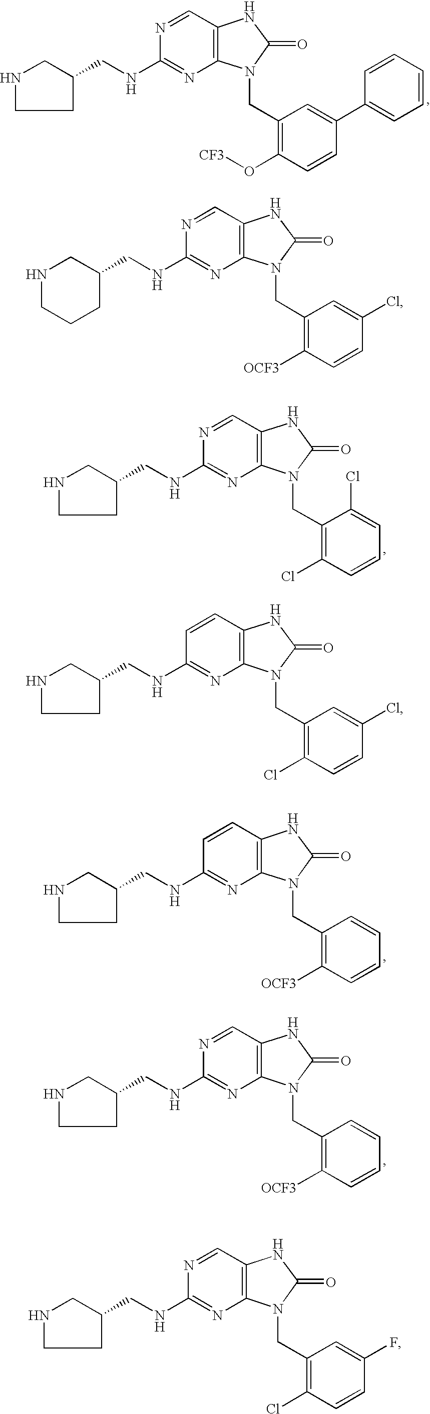

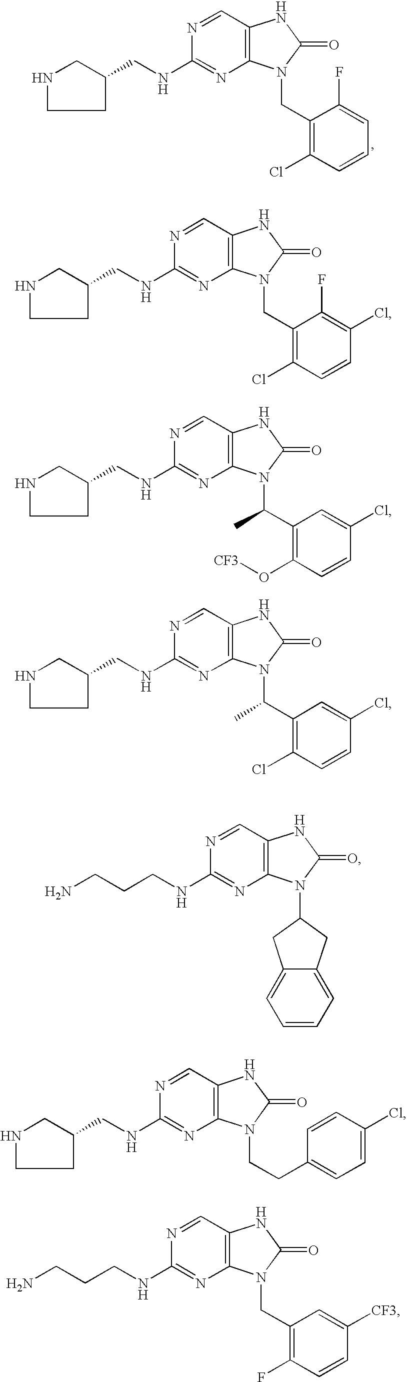

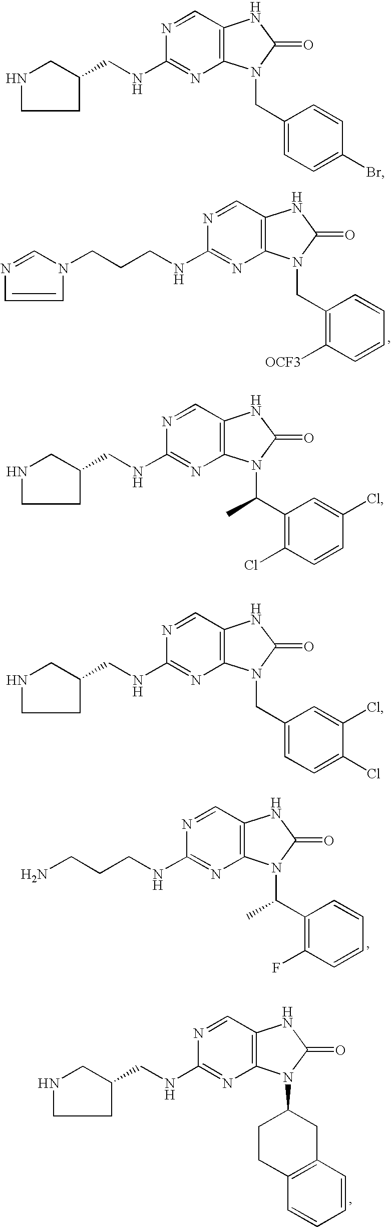

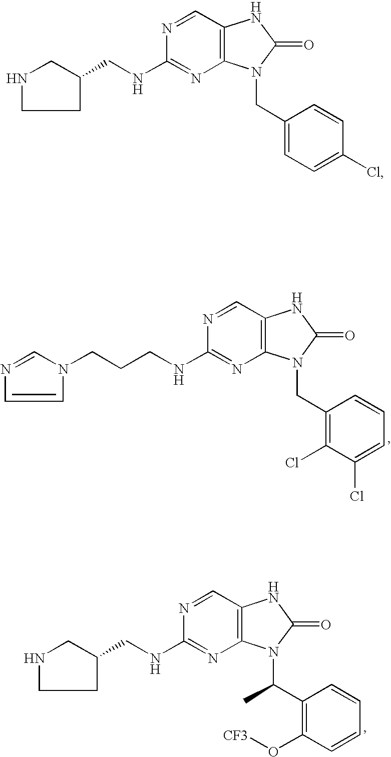

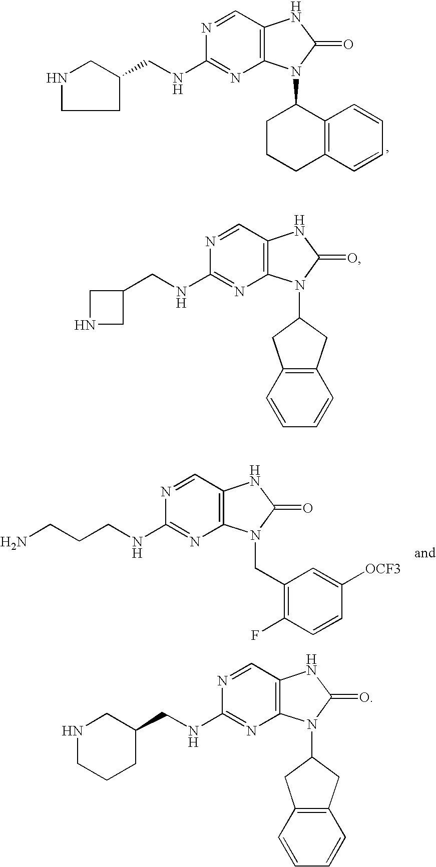

40. A composition comprising a compound according to claim 1 and a pharmaceutically acceptable carrier.

41. A method of treatment of a T-cell mediated disease comprising administering a therapeutically effective amount of a compound according to claim 1.

42. The method of claim 41, wherein the T-cell mediated disease is an autoimmune disease.

43. The method of claim 42, wherein the autoimmune disease is rheumatoid arthritis.

44. The method of claim 42, wherein the autoimmune disease is lupus erythematosus.

45. The method of claim 42, wherein the autoimmune disease is multiple sclerosis.

46. The method of claim 42, wherein the autoimmune disease is psoriasis.

47. The method of claim 41, wherein the T-cell mediated disease is an inflammatory disease.

48. The method of claim 47, wherein the inflammatory disease is asthma.

49. The method of claim 47, wherein the inflammatory disease is inflammatory bowel disease.

50. The method of claim 41, wherein the T-cell mediated disease is transplant rejection.

51. A method of treatment of cancer comprising administering a therapeutically effective amount of a compound according to claim 1.

52. The method of claim 51, wherein the cancer is gastrointestinal cancer.

53. A method of treatment of diabetes comprising administering a therapeutically effective amount of a compound according to claim 1.
